(19) United States
(12) Patent Application Publication
(10) Pub. No.: US 2023/0250121 A1
(43) Pub. Date: Aug. 10, 2023

(54) METHOD FOR PREPARING GLUCOPYRANOSYL DERIVATIVES AND INTERMEDIATES THEREOF

(71) Applicant: SUNSHINE LAKE PHARMA CO., LTD., Dongguan, Guangdong (CN)

(72) Inventors: Zheng GU, Dongguan (CN); Tong QU, Dongguan (CN); Wuyong WU, Dongguan (CN); Weiming HUANG, Dongguan (CN); Daoqian CHEN, Dongguan (CN); Zongyuan ZHANG, Dongguan (CN); Haoxiong QIN, Dongguan (CN); Zheng LI, Dongguan (CN); Weihui YUAN, Dongguan (CN); Shijie NIU, Dongguan (CN); Fei PENG, Dongguan (CN); Tianyun WU, Dongguan (CN)

(73) Assignee: SUNSHINE LAKE PHARMA CO., LTD., Dongguan, Guangdong (CN)

(21) Appl. No.: 18/015,138

(22) PCT Filed: Jul. 7, 2021
(86) PCT No.: PCT/CN2021/104973

§ 371 Date: Jan. 9, 2023

(30) Foreign Application Priority Data
Jul. 8, 2020 (CN) 202010650788.6

(51) Int. Cl. C07H 1/00; C07D 295/108; C07D 295/26; C07D 317/22

(52) U.S. Cl. CPC C07H 1/00; C07D 295/108; C07D 295/26; C07D 317/22

(57) ABSTRACT

A method for preparing glucopyranosyl derivatives as sodium dependent glucose cotransporter (SGLT) inhibitors, an intermediate thereof, and a method for preparing the intermediate.

### CROSS-REFERENCE TO RELATED APPLICATION

This application claims the priority and benefits of Chinese Patent Application No. 202010650788.6, filed with the State Intellectual Property Office of China on Jul. 8, 2020, which is incorporated herein by reference in its entirety.

### FIELD OF THE INVENTION

This present invention pertains to the field of pharmaceutical chemistry, which relates to a method for preparing glucopyranosyl derivatives as sodium dependent glucose cotransporter (SGLT) inhibitors and key intermediates thereof.

### BACKGROUND OF THE INVENTION

The international application PCT/CN2016/079634 (WO 2016173425) discloses compound (I), which can be used as sodium-dependent glucose transporter (SGLT) inhibitors for the treatment of diabetes and diabetes-related diseases, and a preparation method thereof. That application discloses two methods for preparing compound (I) which are as follows:

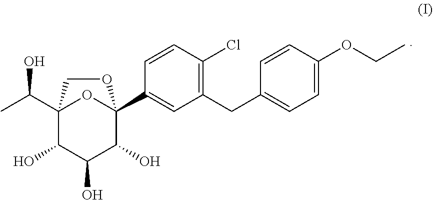 [SMILES: CCOc1ccc(Cc2cc([C@]34OC[C@]([C@@H](C)O)(O3)[C@@H](O)[C@H](O)[C@H]4O)ccc2Cl)cc1]

### Synthesis Route 1:

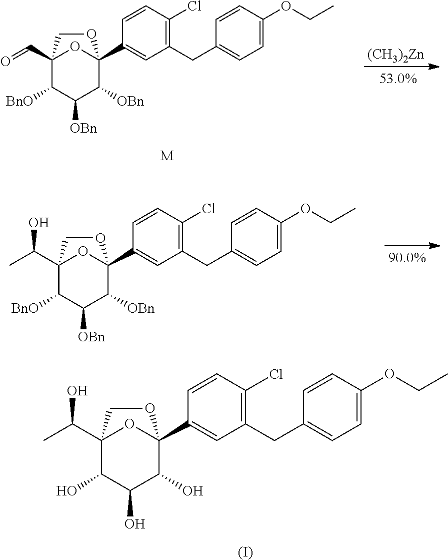

### Synthesis Route 2:

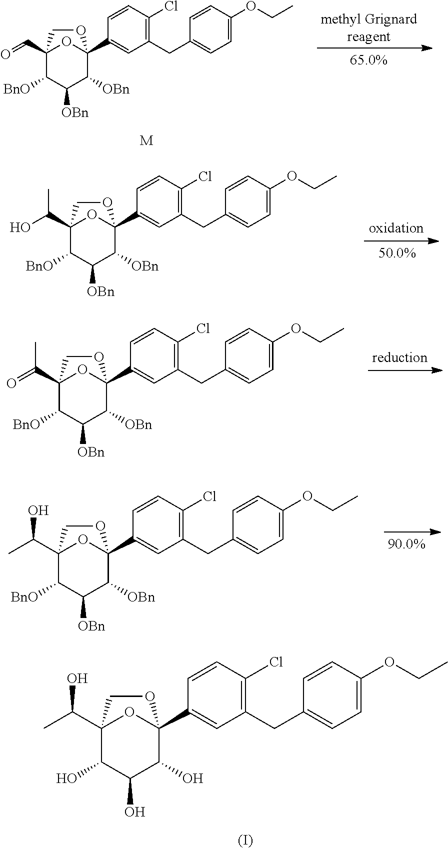

wherein, in synthesis route 1, compound M is used as the starting raw material to obtain compound (I) through an addition reaction with dimethyl zinc and removal of hydroxy protecting group; in synthesis route 2, compound M is used as the starting raw material to obtain compound (I) through an addition reaction with a methyl Grignard reagent, and then through an oxidation reaction, a reduction reaction and removal of the hydroxy protecting group.

Wherein the synthetic route of compound M is as follows:

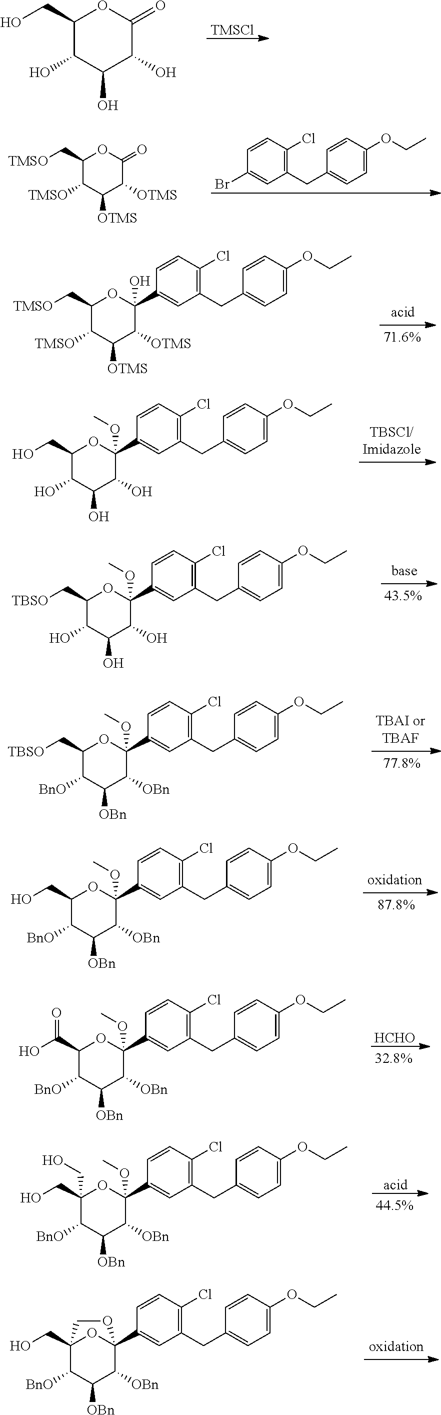

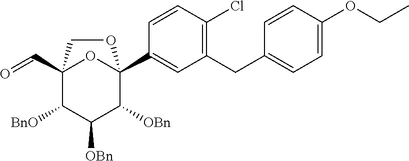 [SMILES: CCOc1ccc(Cc2cc([C@]34OC[C@](C=O)(O3)[C@@H](OCc3ccccc3)[C@H](OCc3ccccc3)[C@H]4OCc3ccccc3)ccc2Cl)cc1]

In both synthetic route 1 and synthetic route 2, two silica gel column chromatography purifications are needed, and the total yield is less than 5%; the requirements for equipment are high, and the synthesis cost is extremely high, and thus they are not suitable for industrial production.

### SUMMARY OF THE INVENTION

In view of the problems existing in the preparation of compound (I) in the prior art, the synthetic route of compound (I) is optimized herein, and a preparation method is provided herein, which is suitable for industrial production. On the one hand, the preparation method has fewer steps, high yield of the intermediate, and can effectively remove the impurities, so that the total yield is greatly improved; specifically, the total yield of the method can reach more than 10%, the optical purity of the obtained product is high, and the production cost is reduced greatly. On the other hand, the purification process does not comprise silica gel column chromatography, the work-up is simple, the purification is easy, and the requirements on equipment is low, and the process is safer, more controllable and simpler.

Specifically, the intermediate compound (VIII) of the present invention can be prepared by the following method.

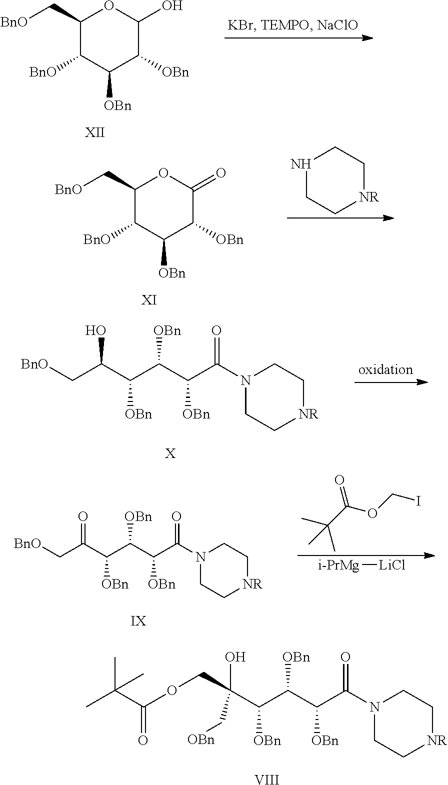

wherein, R is H, C₂₋₈ alkyl (e.g., ethyl, n-propyl, n-butyl, tert-butyl, 2,2-dimethylpropyl), C₄₋₈ alkenyl, allyl, phenyl, benzyl, p-toluenesulfonyl, benzenesulfonyl, 4-bromobenzenesulfonyl, 4-nitrophenyl, 1,3-dichlorophenyl, tert-butoxycarbonyl, triphenylmethyl, bis(4-methoxyphenyl)(phenyl)methyl, diphenylmethyl, N,N-diphenylaminoacyl, pyridyl, benzylsulfonyl, imidazolyl, N,N-dimethylaminosulfonyl, N,N-dimethylaminoacyl or

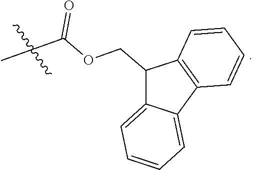 [SMILES: *C(=O)OCC1c2ccccc2-c2ccccc21]

Specifically, the method for preparing the intermediate (VIII) comprises the following steps: first, compound (XII) is oxidized under the oxidation system consisting of sodium hypochlorite, TEMPO and potassium bromide to obtain compound (XI); then, compound (XI) is reacted with substituted or unsubstituted piperazine to obtain compound (X); after that, compound (X) is oxidized to obtain compound (IX); finally, iodomethyl pivalate reacts with isopropylmagnesium chloride lithium chloride or isopropylmagnesium chloride, and then reacts with compound (IX) through a Grignard reaction to obtain compound (VIII).

Other methods can be incorporated in the preparation methods of the present invention as long as compound (VIII) can be prepared.

The present invention relates to a method for preparing compound (I), intermediates thereof and methods for preparing the related intermediates.

On the one hand, the present invention relates to a method for preparing compound (Va) comprising the step of: reacting compound (VIa) with methylating reagent 1 through an addition reaction in the presence of isopropyl titanate to obtain compound (Va),

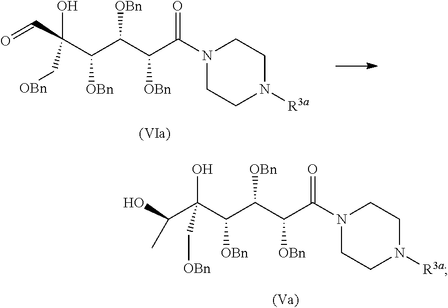

wherein, R^{3a} is C₂₋₈ alkyl, C₄₋₈ alkenyl, allyl, phenyl, benzyl, p-toluenesulfonyl, benzenesulfonyl, 4-bromobenzenesulfonyl, 4-nitrophenyl, 1,3-dichlorophenyl, tert-butoxycarbonyl, triphenylmethyl, bis(4-methoxyphenyl)(phenyl)methyl (i.e.,

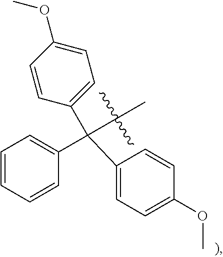 [SMILES: *C(c1ccccc1)(c1ccc(OC)cc1)c1ccc(OC)cc1]

diphenylmethyl, N,N-diphenylaminoacyl, pyridyl, benzylsulfonyl, imidazolyl (such as

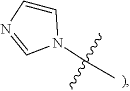 [SMILES: *n1ccnc1]

### N,N-dimethylaminosulfonyl, N,N-dimethylaminoacyl, or

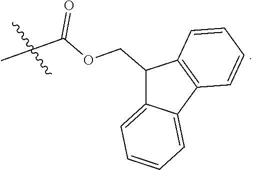 [SMILES: *C(=O)OCC1c2ccccc2-c2ccccc21]

In some embodiments, the reaction is optionally carried out in the presence of a chiral ligand 1, and the chiral ligand 1 is a dihydroxy chiral ligand or a metal ligand.

In other embodiments, the dihydroxy chiral ligand is R-1,1′-bi-2-naphthol ((R)-BINOL), (4R,5R)-2,2-dimethyl-a,a,a′,a′-tetraphenyl-1,3-dioxolane-4,5-dimethanol (TADDOL), (S)-(−)-5,5′,6,6′,7,7′,8,8′-octahydro-1,1′-2-naphthol ((S)-H₈-BINOL) or Salen ligand.

In other embodiments, the metal ligand is a metal-Salen ligand, a metal-BINOL ligand or (1R,2R)-(+)-N,N′-di-p-toluenesulfonyl-1,2-cyclohexanediamine-metal ligand.

In still other embodiments, the metal-Salen ligand is Zn-Salen ligand, Mn-Salen ligand, Ti-Salen ligand or Cr-Salen ligand; preferably, the metal-Salen is Ti-Salen ligand.

In still other embodiments, the metal-BINOL ligand is Zn-BINOL ligand, Mn-BINOL ligand, Ti-BINOL ligand or Cr-BINOL ligand.

In still other embodiments, BINOL of the metal-BINOL ligand is R configuration or S configuration.

In still other embodiments, the (1R,2R)-(+)-N,N′-di-p-toluenesulfonyl-1,2-cyclohexanediamine-metal ligand is (1R,2R)-(+)-N,N′-di-p-toluenesulfonyl-1,2-cyclohexanediamine-Zn ligand, (1R,2R)-(+)-N,N′-di-p-toluenesulfonyl-1,2-cyclohexanediamine-Mn ligand, (1R,2R)-(+)-N,N′-di-p-toluenesulfonyl-1,2-cyclohexanediamine-Ti ligand or (1R,2R)-(+)-N,N′-di-p-toluenesulfonyl-1,2-cyclohexanediamine-Cr ligand.

In still other embodiments, the Salen ligand, Zn-Salen ligand, Mn-Salen ligand, Ti-Salen ligand and Cr-Salen ligand are respectively preferably selected from the following structures:

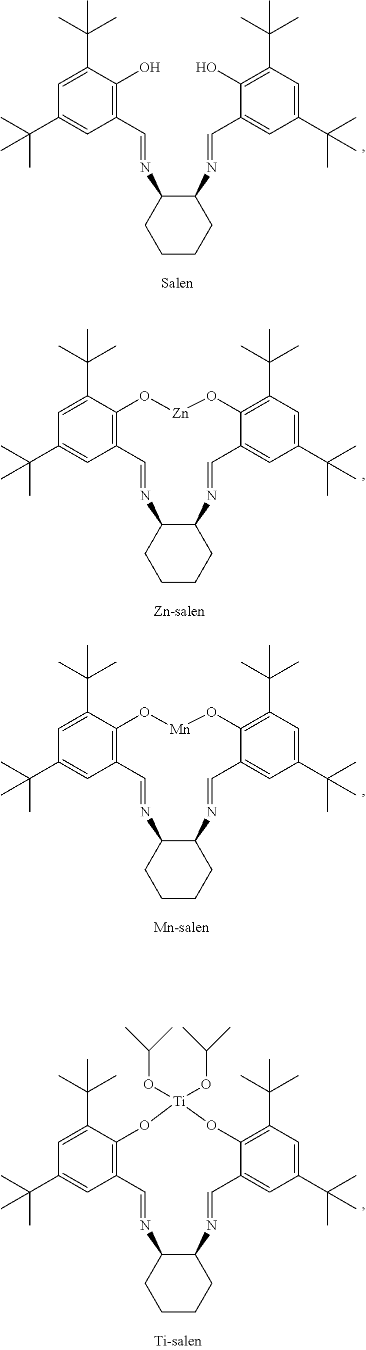

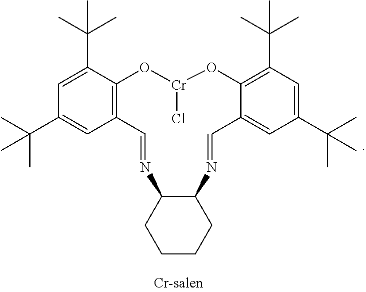

In still other embodiments, the amount of substance of metal-Salen ligand is 0.1 to 1.0 time that of compound (VIa).

In still other embodiments, the amount of substance of Salen ligand is 0.05 to 1.0 time that of compound (VIa).

In other embodiments, the chiral ligand 1 is R-1,1′-Bi-2-naphthol ((R)-BINOL), (4R,5R)-2,2-dimethyl-a,a,a′,a′-tetraphenyl-1,3-dioxolane-4,5-dimethanol (TADDOL), (S)-(−)-5,5′,6,6′,7,7′,8,8′-octahydro-1,1′-2-naphthol ((S)-H₈-BINOL), Salen ligand, metal-Salen ligand, metal-BINOL ligand or (1R,2R)-(+)-N,N′-di-p-toluenesulfonyl-1,2-cyclohexanediamine-metal ligand.

In still other embodiments, the amount of substance of R-1,1′-Bi-2-naphthol is 0.01 to 0.9 times that of compound (VIa); preferably, the amount of substance of R-1,1′-Bi-2-naphthol is 0.05 to 0.2 times that of compound (VIa); preferably, the amount of substance of R-1,1′-Bi-2-naphthol is 0.05, 0.1 or 0.2 times that of compound (VIa).

In still other embodiments, the amount of substance of Ti-BINOL ligand is 0.01 to 0.20 times that of compound (VIa); preferably, the amount of substance of Ti-BINOL ligand is 0.01 to 0.10 times that of compound (VIa).

In still other embodiments, the amount of substance of Salen ligand is 1.0 time or less that of compound (VIa); preferably, the amount of substance of Salen ligand is 0.2 times or less that of compound (VIa).

In still other embodiments, the amount of substance of isopropyl titanate is 0.5 to 8.0 times that of compound (VIa); preferably, the amount of substance of isopropyl titanate is 1.0 to 5.0 times that of compound (VIa); preferably, the amount of substance of isopropyl titanate is 1.4 to 4.0 times that of compound (VIa); preferably, the amount of substance of isopropyl titanate is 2.0 to 4.0 times that of compound (VIa); preferably, the amount of substance of isopropyl titanate is 3.9 times that of compound (VIa); preferably, the amount of substance of isopropyl titanate is 1.4, 2.0, 3.0 or 4.0 times that of compound (VIa).

In some embodiments, the methylating agent 1 is methylmagnesium bromide, methylmagnesium chloride, methyllithium, trimethylaluminum or dimethyl zinc.

In other embodiments, the amount of substance of methylating reagent 1 is 3.0 to 6.0 times that of compound (VIa); preferably, the amount of substance of methylating reagent 1 is 4.0 to 6.0 times that of compound (VIa); preferably, the amount of substance of methylating reagent 1 is 4.0 to 5.0 times that of compound (VIa).

In still other embodiments, the amount of substance of dimethyl zinc is 3.0 to 6.0 times that of compound (VIa); preferably, the amount of substance of dimethyl zinc is 4.0 to 6.0 times that of compound (VIa).

In still other embodiments, the amount of substance of methylmagnesium bromide is 3.0 to 6.0 times that of compound (VIa); preferably, the amount of substance of methylmagnesium bromide is 4.0 to 6.0 times that of compound (VIa); preferably, the amount of substance of methylmagnesium bromide is 4.0 to 5.0 times that of compound (VIa); preferably, the amount of substance of methylmagnesium bromide is 4.0, 4.5, 5.0 or 6.0 times that of compound (VIa).

As described in the present invention, the methylmagnesium bromide can be a solution of methylmagnesium bromide in 2-methyltetrahydrofuran with a concentration of 3 mol/L.

In some embodiments, the reaction of compound (VIa) and methylating reagent 1 is carried out in an organic solvent, and the organic solvent is dichloromethane, tetrahydrofuran, methyltetrahydrofuran (e.g., 2-methyltetrahydrofuran), methyl tert-butyl ether, toluene, o-xylene, p-xylene, meta-xylene or any combination thereof.

In still other embodiments, in the reaction of compound (VIa) and methylating reagent 1, the methylating reagent 1 is methylmagnesium bromide, and the reaction solvent is dichloromethane, tetrahydrofuran, methyltetrahydrofuran (e.g., 2-methyltetrahydrofuran), methyl tert-butyl ether, toluene, o-xylene, p-xylene, meta-xylene or any combination thereof; or, in the reaction of compound (VIa) and methylating reagent 1, the methylating reagent 1 is dimethyl zinc, and the reaction solvent is dichloromethane, tetrahydrofuran, methyl tetrahydrofuran, methyl tert-butyl ether, toluene, o-xylene, p-xylene, meta-xylene or any combination thereof.

In some embodiments, the reaction of compound (VIa) and methylating reagent 1 is carried out under room temperature conditions. In some embodiments, in the reaction of compound (VIa) and methylating reagent 1, the reaction temperature is 10° C.˜40° C.; preferably, the reaction temperature is 20° C.˜35° C.; preferably, the reaction temperature is 20° C.˜32° C.; preferably, the reaction temperature is 20° C.˜30° C.

In some embodiments, in the reaction of compound (VIa) and methylating reagent 1, compound (VIa) can be added by dropping; optionally, during the dropping of compound (VIa), the reaction system is kept at a certain temperature. Preferably, the temperature of the reaction system during the dropping process is −20° C.˜ 25° C.; preferably, the temperature of the reaction system during the dropping process is −10° C.˜ 0° C.; preferably, the temperature of the reaction system during the dropping process is −5° C.˜0° C.

As described in the present invention, in the reaction, a new chiral center can be introduced through an asymmetric addition reaction of an aldehyde group with methylating reagent, and a product with high ee value and intermediate (Va) with a high yield can be obtained through selection and optimization of methylating reagents, chiral ligands and/or other conditions. Specifically, the type and dosage of methylating reagent have different effects on the reaction. When methylating reagent is methylmagnesium bromide, and the amount of methylmagnesium bromide is 4.0 to 6.0 equivalents, the reaction is complete and the ee value of the product is high.

In some embodiments, compound (VIa) can be prepared by the following method:

step a: reacting compound (VIIIa) in the presence of alkaline reagent 1 through a hydrolysis reaction to obtain compound (VIIa),

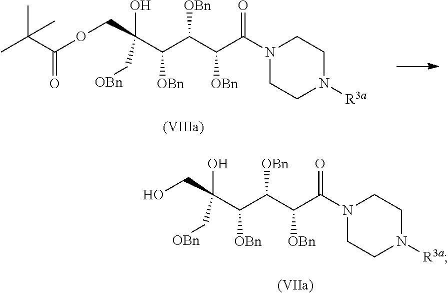

step b: reacting compound (VIIa) in the presence of oxidant 1 through an oxidation reaction to obtain compound (VIa),

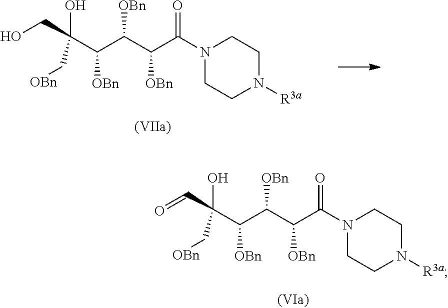

wherein, R^{3a} is as described in the present invention.

In other embodiments, the alkaline reagent 1 in step a is sodium methoxide, sodium ethoxide, sodium tert-butoxide, potassium tert-butoxide, sodium carbonate, potassium carbonate, cesium carbonate, sodium bicarbonate, sodium hydroxide or potassium hydroxide.

In other embodiments, the solvent used in step a is dichloromethane, toluene, dichloroethane, methyl tert-butyl ether, xylene (e.g., o-xylene, p-xylene, meta-xylene), dimethyl sulfoxide, methanol, ethanol, tetrahydrofuran, methyltetrahydrofuran (e.g., 2-methyltetrahydrofuran) or any combination thereof.

In still other embodiments, the solvent used in step a is toluene or a mixed solvent of toluene and dimethyl sulfoxide. Preferably, the solvent used in step a is a mixed solvent of toluene and dimethyl sulfoxide, wherein the volume ratio of toluene and dimethyl sulfoxide is (20:1)˜(25:1).

In other embodiments, the reaction temperature in step a is −15° C.˜30° C.; preferably, the reaction temperature in step a is −15° C.˜5° C.; preferably, the reaction temperature in step a is −15° C.˜0° C.

In still other embodiments, the alkaline reagent 1 in step a is sodium methoxide, sodium ethoxide, sodium tert-butoxide or potassium tert-butoxide; the solvent used in step a is toluene or a mixed solvent of toluene and dimethyl sulfoxide; and the reaction temperature in step a is −15° C.˜0° C. Preferably, the solvent is a mixed solvent of toluene and dimethyl sulfoxide, wherein the volume ratio of toluene and dimethyl sulfoxide is (20:1)˜(25:1).

In other embodiments, the oxidant 1 in step b is sodium hypochlorite, 2,2,6,6-tetramethylpiperidine oxide, sulfur trioxide pyridine, oxygen, ozone, Dess-Martin oxidizer, iron nitrate, 2-iodoyl benzoic acid or iodine.

In other embodiments, the solvent used in step b is toluene, methyl tert-butyl ether, tetrahydrofuran, methyltetrahydrofuran (e.g., 2-methyltetrahydrofuran), dimethyl sulfoxide, dichloromethane, dichloroethane or any combination thereof.

In other embodiments, the reaction temperature in step b is −10° C.˜30° C.; preferably, the reaction temperature in step b is −5° C.˜5° C.; preferably, the reaction temperature in step b is −5° C., −5° C.˜0° C. or 0° C.˜5° C.

In other embodiments, the reaction of step b is carried out in the presence of an alkaline reagent b, wherein the alkaline reagent b is N,N-diisopropylethylamine, triethylamine, pyridine, 4-dimethylaminopyridine, N-methylmorpholine, 1,8-diazabicycloundec-7-ene or tetramethylethylenediamine.

In other embodiments, compound (VIa) can optionally be further purified by the following steps:

step (b-1): reacting compound (VIa) with sodium bisulfite to obtain compound (VIa-1);

step (b-2): reacting compound (VIa-1) in the presence of an alkaline reagent 2 to obtain purified compound (VIa);

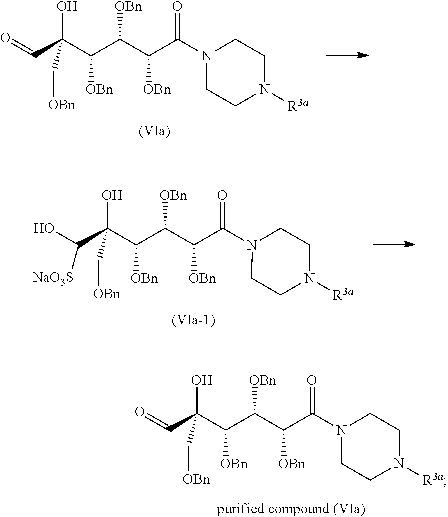

wherein, R^{3a} is as described herein.

In still other embodiments, the reaction solvent in step (b-1) is water.

In still other embodiments, the reaction solvent in step (b-2) is toluene, dichloromethane, tetrahydrofuran, methyltetrahydrofuran (e.g., 2-methyltetrahydrofuran), methanol, ethanol, n-heptane, methyl tert-butyl ether, diethyl ether, isopropyl ether or any combination thereof.

In still other embodiments, the alkaline reagent 2 in step (b-2) is potassium carbonate, sodium carbonate, sodium hydroxide, sodium bicarbonate or cesium carbonate.

In other embodiments, the method for preparing compound (VIa) of the present invention further comprises a purification method of compound (VIIIa): adding the material containing compound (VIIIa) into solvent A, and then adding solvent B to precipitate a solid compound (VIIIa). Specifically, the solvent A is methanol, ethanol, isopropanol, n-butanol, tert-butanol, acetone, toluene, xylene (e.g., o-xylene, p-xylene, meta-xylene), ethyl acetate, dichloromethane, methyl tert-butyl ether, diethyl ether, isopropyl ether, anisole or any combination thereof; and the solvent B is n-heptane, n-hexane, cyclohexane, petroleum ether, water or any combination thereof. The material containing compound (VIIIa) may be a mixture containing a certain amount of compound (VIIIa) or a solution thereof (e.g., a solution in tetrahydrofuran); generally, the material is a compound (VIIIa) with a certain purity or a solution thereof. Specifically, the material containing compound (VIIIa) refers to a crude product of compound (VIIIa) or a solution thereof (e.g., a solution in tetrahydrofuran) prepared according to the preparation method of compound (VIII).

In still other embodiments, the crystallization temperature of the solid compound (VIIIa) is 40° C.˜10° C.; preferably, the crystallization temperature of the solid compound (VIIIa) is 30° C.˜10° C.

In still other embodiments, during the purification process of compound (VIIIa), the compound (VIIIa) needed to be further purified can be dissolved in a solvent by means such as stirring, ultrasound or heating. Specifically, the dissolution temperature of the material containing compound (VIIIa) is room temperature to 60° C.; preferably, the dissolution temperature during the purification process is room temperature, 35° C. or 60° C.

In still other embodiments, the solvent A is toluene, and the solvent B is n-heptane; or the solvent A is toluene, and the solvent B is n-hexane; or the solvent A is ethanol, and the solvent B is water; or the solvent A is isopropanol, and the solvent B is water; or the solvent A is tert-butanol, and the solvent B is water; or the solvent A is acetone, and the solvent B is water; or the solvent A is ethanol, and the solvent B is n-heptane; or the solvent A is ethanol, and the solvent B is n-hexane; or the solvent A is ethanol, and the solvent B is cyclohexane; or the solvent A is ethyl acetate, and the solvent B is n-heptane; or the solvent A is ethyl acetate, and the solvent B is n-hexane; or the solvent A is methyl tert-butyl ether, and the solvent B is n-heptane.

In still other embodiments, the solvent A is toluene, and the solvent B is cyclohexane.

In still other embodiments, the solvent A is toluene, and the solvent B is n-heptane, wherein the volume ratio of toluene and n-heptane is (1:3)˜(1:10); preferably, the volume ratio of toluene and n-heptane is (1:3)˜(1:8); preferably, the volume ratio of toluene and n-heptane is (1:3)˜(1:5); preferably, the volume ratio of toluene and n-heptane is (1:5)˜(1:8); preferably, the volume ratio of toluene and n-heptane is (1:8), (1:7), (1:6), (1:5), (1:3) or (1:4).

In still other embodiments, the solvent A is ethanol, and the solvent B is water, wherein the volume ratio of ethanol and water is (1:1)˜(3:1).

In still other embodiments, the solvent A is isopropanol, and the solvent B is water, wherein the volume ratio of isopropanol and water is (1:1)˜(3:1).

In still other embodiments, the solvent A is acetone, and the solvent B is water, wherein the volume ratio of acetone and water is (1:1)˜(10:1); preferably, the volume ratio of acetone and water is (2:1)˜(8:1); preferably, the volume ratio of acetone and water is (4:1)˜(6:1).

In still other embodiments, the solvent A is ethanol, and the solvent B is n-heptane, wherein the volume ratio of ethanol and n-heptane is (1:1)˜(3:1).

In still other embodiments, the solvent A is ethanol, and the solvent B is n-hexane, wherein the volume ratio of ethanol and n-hexane is (1:1)˜(3:1).

In still other embodiments, the solvent A is ethanol, and the solvent B is cyclohexane, wherein the volume ratio of ethanol and cyclohexane is (1:1)˜(3:1).

In still other embodiments, the solvent A is ethyl acetate, and the solvent B is n-heptane, wherein the volume ratio of ethyl acetate and n-heptane is (1:1)˜(1:10); preferably, the volume ratio of ethyl acetate and n-heptane is (1:1)˜(1:5); preferably, the volume ratio of ethyl acetate and n-heptane is (1:2)˜(1:3); preferably, the volume ratio of ethyl acetate and n-heptane is (1:2), (1:3), (1:4), (1:5) or (1:6).

In still other embodiments, the solvent A is methyl tert-butyl ether, and the solvent B is n-heptane, wherein the volume ratio of methyl tert-butyl ether and n-heptane is (1:3)˜(1:10); preferably, the volume ratio of methyl tert-butyl ether and n-heptane is (1:3), (1:4), (1:5) or (1:6).

In still other embodiments, the solvent A is ethyl acetate, and the solvent B is n-hexane, wherein the volume ratio of ethyl acetate and n-hexane is (1:1)˜(1:10); preferably, the volume ratio of ethyl acetate and n-hexane is (1:1)˜(1:5); preferably, the volume ratio of ethyl acetate and n-hexane is (1:3), (1:4), (1:5) or (1:6).

In still other embodiments, the solvent A is toluene, and the solvent B is cyclohexane, wherein the volume ratio of toluene and cyclohexane is (1:3)˜(1:10); preferably, the volume ratio of toluene and cyclohexane is (1:3), (1:4), (1:5) or (1:6).

In still other embodiments, the solvent A is toluene, and the solvent B is n-hexane, wherein the volume ratio of toluene and n-hexane is (1:3)˜(1:10); preferably, the volume ratio of toluene and n-hexane is (1:3)˜(1:8); preferably, the volume ratio of toluene and n-hexane is (1:5)˜(1:8); preferably, the volume ratio of toluene and n-hexane is (1:3), (1:4), (1:5) or (1:6).

In still other embodiments, the solvent A is methyl tert-butyl ether, the solvent B is n-heptane, wherein the volume ratio of methyl tert-butyl ether and n-heptane is (1:1)˜(1:10); preferably, the volume ratio of methyl tert-butyl ether and n-heptane is (1:1)˜(1:5); preferably, the volume ratio of methyl tert-butyl ether and n-heptane is (1:2)˜(1:3); preferably, the volume ratio of methyl tert-butyl ether and n-heptane is (1:2), (1:3), (1:4), (1:5) or (1:6).

In other embodiments, the compound (VIa) obtained in step b needs to be further dried.

In other embodiments, the product obtained in step b needs to be further washed and dried.

In other embodiments, in step b, the water content of compound (VIa) is ≤1%; preferably, the water content of compound (VIa) is ≤0.6%.

As described in the present invention, in step b, different oxidants have different effects on the reaction. Through a large number of screening tests, it is found that when the oxidant is sulfur trioxide pyridine, the reaction yield is high, and the obtained compound (VIa) has high purity. In the work-up process, it needs to be washed for many times (such as washing with water or brine for many times). After washing, the product needs to be dried, or it is easy to deteriorate because of an excessive water content.

In some embodiments, the compound (Va) of the present invention has a structure represented by formula (Vb).

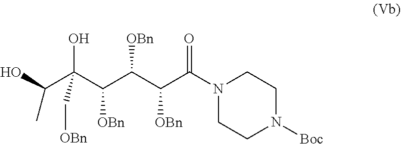 [SMILES: C[C@@H](O)[C@](O)(COCc1ccccc1)[C@@H](OCc1ccccc1)[C@H](OCc1ccccc1)[C@@H](OCc1ccccc1)C(=O)N1CCN(C(=O)OC(C)(C)C)CC1]

Further, the present invention relates to a method for preparing compound (Vb), wherein the method comprises the step of: reacting compound (VIb) with methylating reagent 1 through an addition reaction in the presence of isopropyl titanate to obtain compound (Vb);

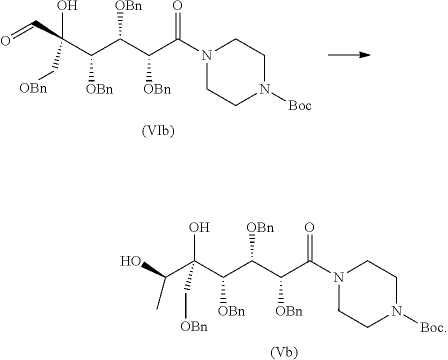

Preferably, the compound (Vb) can be prepared according to the preparation method of compound (Va) of the present invention.

On the other hand, the present invention relates to a method for preparing compound (Vc) which is method M comprising: reacting compound (VIb) with methylating reagent 1 through an addition reaction in the presence of isopropyl titanate to obtain compound (Vb); and continuing to react to remove the tert-butoxycarbonyl protecting group from compound (Vb) to obtain compound (Vc);

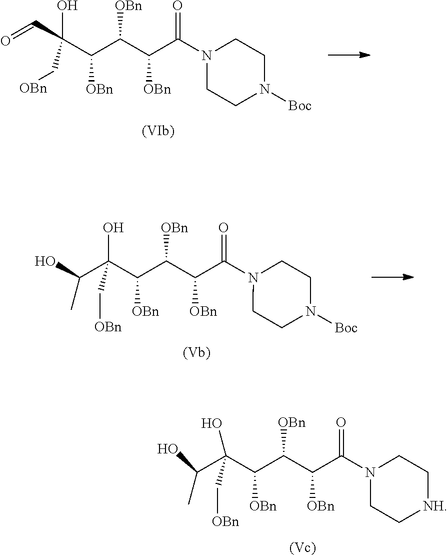

In some embodiments, in the addition reaction of compound (VIb) and methylating reagent 1, the methylating reagent 1 is methylmagnesium bromide or dimethyl zinc; wherein the amount of substance of methylmagnesium bromide is 4.0 to 5.0 times that of compound (VIb); and the amount of substance of dimethyl zinc is 4.0 to 6.0 times that of compound (VIb).

In some embodiments, in the addition reaction of compound (VIb) and methylating reagent 1, the amount of substance of isopropyl titanate is 1.0 to 5.0 times that of compound (VIb); preferably, the amount of substance of isopropyl titanate is 1.4 to 4.0 times that of compound (VIb); preferably, the amount of substance of isopropyl titanate is 2.0 to 4.0 times that of compound (VIb).

In some embodiments, in the addition reaction of compound (VIb) and methylating reagent 1, the amount of substance of isopropyl titanate is 2.0 to 4.0 times that of compound (VIb), the methylating reagent 1 is methylmagnesium bromide, and the amount of substance of methylmagnesium bromide is 4.0 to 5.0 times that of compound (VIb).

In some embodiments, in the addition reaction of compound (VIb) and methylating reagent 1, the amount of substance of isopropyl titanate is 1.4 to 2.0 times that of compound (VIb), the methylating reagent 1 is dimethyl zinc, and the amount of substance of dimethyl zinc is 4.0 to 6.0 times that of compound (VIb).

In some embodiments, in the addition reaction of compound (VIb) and methylating reagent 1, the solvent of the addition reaction is dichloromethane, tetrahydrofuran, methyltetrahydrofuran (e.g., 2-methyltetrahydrofuran), methyl tert-butyl ether, toluene, o-xylene, p-xylene, meta-xylene or a combination thereof. Preferably, the solvent of the addition reaction is dichloromethane.

In some embodiments, in the addition reaction of compound (VIb) and methylating reagent 1, the reaction temperature is 10° C.˜40° C.; preferably, the reaction temperature is 20° C.˜32° C.; preferably, the reaction temperature is 20° C.˜30° C.

In some embodiments, in the addition reaction of compound (VIb) and methylating reagent 1, compound (VIb) is added by dropping; and optionally, the reaction system is kept at a certain temperature when compound (VIb) is added. Preferably, the temperature of the reaction system during the dropping process is −20° C.˜25° C. Preferably, in the addition reaction, the temperature of the reaction system during the dropping process is −10° C.˜0° C. Preferably, in the addition reaction, the temperature of the reaction system during the dropping process is −5° C.˜0° C.

In some embodiments, the addition reaction of compound (VIb) and methylating reagent 1 is optionally carried out in the presence of a chiral ligand 1; and the chiral ligand 1 is R-1,1′-Bi-2-naphthol, Salen ligand, metal-Salen ligand or metal-BINOL ligand, wherein the metal can be Zn, Mn, Cr or Ti.

In other embodiments, the amount of substance of R-1,1′-Bi-2-naphthol is 0.05 to 0.2 times that of compound (VIb); the amount of substance of Salen ligand is 0.2 times or less that of compound (VIb).

In some embodiments, the compound (VIb) can be prepared according to the preparation method of compound (VIa) of the present invention. Specifically, compound (VIb) of the present invention can be prepared by the following method:

Step a-1: reacting compound (VIIIb) in the presence of alkaline reagent 1 through a hydrolysis reaction to obtain compound (VIIb),

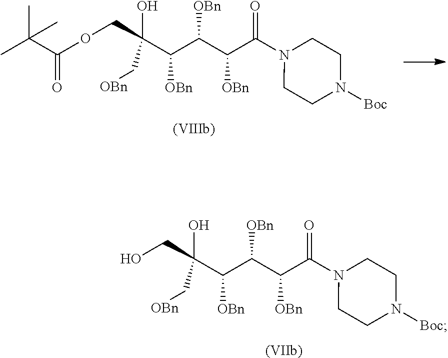

Step b-1: reacting compound (VIIb) in the presence of oxidant 1 through an oxidation reaction to obtain compound (VIb),

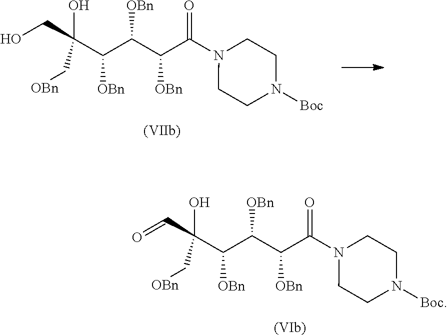

In other embodiments, the alkaline reagent 1 in step a-1 is sodium methoxide, sodium ethoxide, sodium tert-butoxide, potassium tert-butoxide, sodium carbonate, potassium carbonate, cesium carbonate, sodium bicarbonate, sodium hydroxide or potassium hydroxide.

In other embodiments, the solvent used in step a-1 is dichloromethane, toluene, dichloroethane, methyl tert-butyl ether, xylene (e.g., o-xylene, p-xylene, meta-xylene), dimethyl sulfoxide, methanol, ethanol, tetrahydrofuran, 2-methyltetrahydrofuran or any combination thereof.

In other embodiments, the reaction temperature of step a-1 is −15° C.˜30° C.; preferably, the reaction temperature of step a-1 is −15° C.˜5° C.; preferably, the reaction temperature of step a-1 is −15° C.˜0° C.

In other embodiments, the alkaline reagent 1 in step a-1 is sodium methoxide, sodium ethoxide, sodium tert-butoxide or potassium tert-butoxide; the solvent used in step a-1 is toluene or a mixed solvent of toluene and dimethyl sulfoxide; and the reaction temperature of step a-1 is −15° C.˜0° C.

In other embodiments, the solvent used in step a-1 is a mixed solvent of toluene and dimethyl sulfoxide, wherein the volume ratio of toluene and dimethyl sulfoxide is (20:1)˜(25:1).

In other embodiments, the oxidant 1 in step b-1 is sodium hypochlorite, 2,2,6,6-tetramethylpiperidine oxide, sulfur trioxide pyridine, oxygen, ozone, Dess-Martin oxidant, iron nitrate, 2-iodoyl benzoic acid or iodine.

In other embodiments, the solvent used in step b-1 is toluene, methyl tert-butyl ether, tetrahydrofuran, methyltetrahydrofuran (2-methyltetrahydrofuran), dimethyl sulfoxide, dichloromethane, dichloroethane or any combination thereof.

In other embodiments, the reaction temperature of step b-1 is −10° C.˜30° C.; preferably, the reaction temperature of step b-1 is −5° C.˜5° C.; preferably, the reaction temperature of step b-1 is −5° C., −5° C.˜0° C. or 0° C.˜5° C.

In other embodiments, step b-1 is carried out in the presence of an alkaline reagent b, and the alkaline reagent b is N,N-diisopropylethyl amine, triethylamine, pyridine, 4-dimethylaminopyridine, N-methylmorpholine, 1,8-diazabicycloundec-7-ene or tetramethylethylenediamine.

In other embodiments, the method for preparing compound (VIb) also comprises a purification method of compound (VIIIb), wherein the purification method is the same as or similar to the purification method of compound (VIIIa) of the present invention. Specifically, the purification method of compound (VIIIb) comprises: adding the material containing compound (VIIIb) into solvent A, and then adding solvent B to precipitate a solid compound (VIIIb). Wherein, the solvent A and the solvent B independently are as described in the present invention.

In still other embodiments, in the purification method, the crystallization temperature of the solid compound (VIIIb) is 40° C.˜10° C.; preferably, the crystallization temperature of the solid compound (VIIIb) is 30° C.˜10° C.

In some embodiments, the method for removing the tert-butoxycarbonyl group comprises: directly increasing the temperature of the addition reaction to 25° C.˜50° C.; preferably, directly increasing the temperature of the addition reaction to 30° C.˜40° C.; preferably, directly increasing the temperature of the addition reaction to 30° C.˜35° C.

In other aspect, the present invention relates to a method for preparing compound (I), comprising:

Step (A): reacting compound (III) or a salt thereof with compound (IV) through a coupling reaction to obtain compound (II),

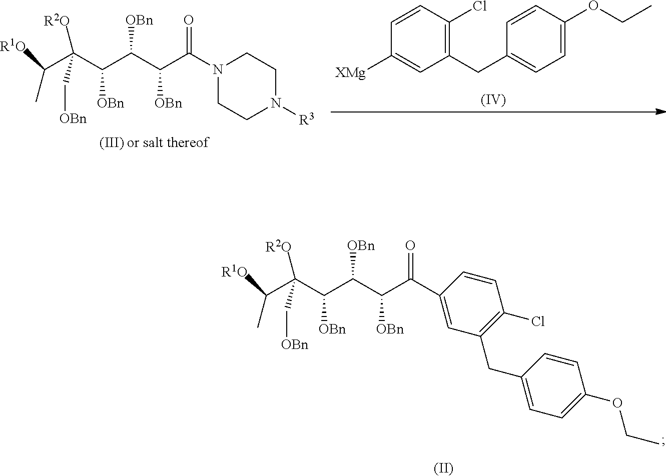

Step (B): reacting compound (II) through hydrogenation reduction and ring closure under acidic condition to obtain compound (I),

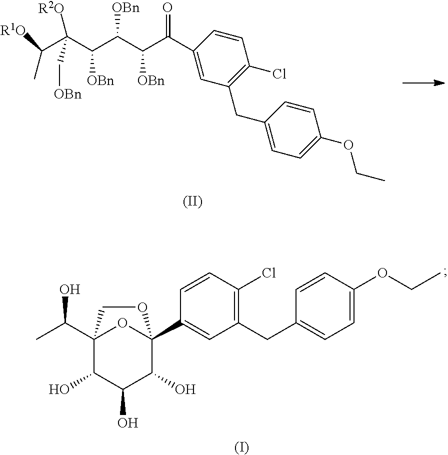

wherein,

X is Cl, Br or I;

R³ is benzyl, C₂₋₈ alkyl (e.g., ethyl, n-propyl, n-butyl, tert-butyl, or 2,2-dimethylpropyl), C₄₋₈ alkenyl, allyl, phenyl, p-toluenesulfonyl, benzenesulfonyl, 4-bromobenzenesulfonyl, 4-nitrophenyl, 1,3-dichlorophenyl, triphenylmethyl, bis(4-methoxyphenyl)(phenyl) methyl, diphenylmethyl, N,N-diphenylaminoacyl, pyridyl, benzylsulfonyl, imidazolyl, N,N-dimethylaminosulfonyl, N,N-dimethylaminoacyl or

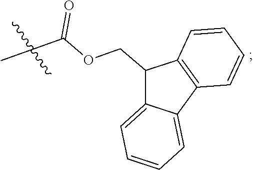 [SMILES: *C(=O)OCC1c2ccccc2-c2ccccc21]

each R¹ and R² is independently benzyl, triphenylmethyl, p-methoxybenzyl, tert-butyldimethylsilyl, trimethylsilyl, tert-butyldiphenylsilyl, triethyl silyl, triisopropylsilyl, benzyloxycarbonyl, 2-(trimethylsilyl)ethoxymethyl, dihydropyranyl, bromopropenyl, ethylformyl, acetyl or benzoyl;

or, R¹ and R² together with —OCHCHO— to which they are attached form

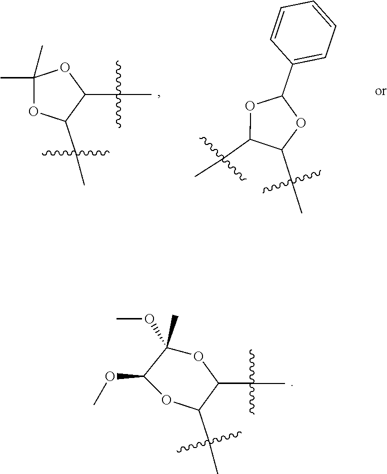

In some embodiments, the salt of compound (III) is methyl quaternary ammonium salt, benzyl quaternary ammonium salt, ethyl quaternary ammonium salt, oxalate, citrate, hydrochloride, phosphate or acetate.

In some embodiments, the acidic condition in step (B) refers to the reaction reacts in the presence of an acid, and the acid is hydrochloric acid, perchloric acid, sulfuric acid, nitric acid, formic acid or acetic acid.

In some embodiments, the hydrogenation reduction reaction is carried out in the presence of a catalyst, and the catalyst is palladium/carbon, palladium hydroxide/carbon, platinum/carbon, Raney nickel or palladium chloride. In other embodiments, the mass ratio of the catalyst and compound (II) is (0.02:1)˜(0.8:1); preferably, the mass ratio of the catalyst and compound (II) is (0.1:1)˜(0.6:1); preferably, the mass ratio of the catalyst and compound (II) is (0.05:1)˜(0.2:1).

In some embodiments, in step (A), the amount of substance of compound (IV) is 1.0 to 1.5 times that of compound (III) or a salt thereof; preferably, in step (A), the amount of substance of compound (IV) is 1.1 to 1.4 times that of compound (III) or a salt thereof; preferably, in step (A), the amount of substance of compound (IV) is 1.2 to 1.4 times that of compound (III) or a salt thereof; preferably, in step (A), the amount of substance of compound (IV) is 1.1 to 1.3 times that of compound (III) or a salt thereof.

In some embodiments, the reaction solvent of step (A) is tetrahydrofuran, methyltetrahydrofuran (e.g., 2-methyltetrahydrofuran), methyl tert-butyl ether, toluene, dichloromethane or any combination thereof.

In some embodiments, the reaction temperature in step (A) is 10° C.˜40° C.; preferably, the reaction temperature in step (A) is 20° C.˜30° C.

In some embodiments, the compound (III) can be prepared by the following method A, method A:

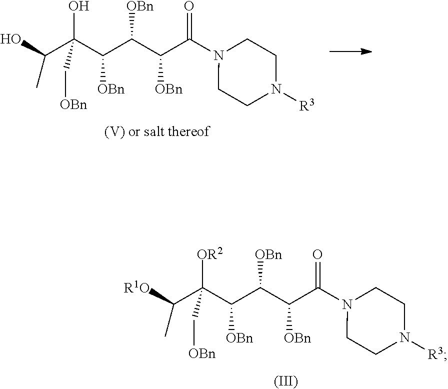

wherein, the method A comprises the step of: reacting compound (V) or a salt thereof with a hydroxyl protecting reagent 1 to obtain compound (III);

each R¹ and R² is independently as described herein.

In some embodiments, the compound (V) can be prepared according to the preparation method of compound (Va) of the present invention.

In other embodiments, the compound (V) can also be prepared by the following method: reacting compound (Vc) with R³X¹ to obtain compound (V),

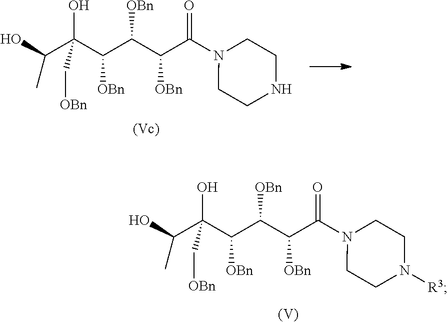

wherein, X¹ is Cl, Br or I, and R³ is as described in the present invention.

In still other embodiments, the reaction of compound (Vc) and R³X¹ of the present invention is carried out in the presence of a base. Preferably, the base includes but is not limited to triethylamine, pyridine, DMAP and the like.

Preferably, the compound (Vc) can be prepared by the method described herein.

In other embodiments, the hydroxyl protecting reagent 1 is 2,2-dimethoxypropane, benzaldehyde dimethyl acetal, trimethylchlorosilane or tert-butyldimethylchlorosilane.

In other embodiments, the reaction solvent of method A is toluene, 1,2-dichloroethane, dichloromethane, methyl tert-butyl ether, isopropyl ether, tetrahydrofuran, 2-methyltetrahydrofuran or any combination thereof.

In other embodiments, the reaction temperature of method A is 0° C.˜40° C.; preferably, the reaction temperature of method A is 10° C.˜40° C.; preferably, the reaction temperature of method A is 8° C.˜30° C.

In other embodiments, the reaction of method A is optionally carried out in the presence of methanesulfonic acid.

In other embodiments, the compound (V) provided herein has one of the following structures:

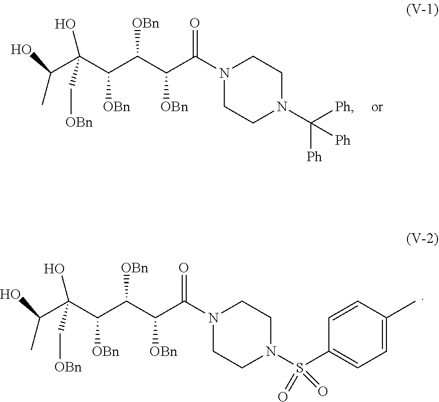 [SMILES: C[C@@H](O)[C@](O)(COCc1ccccc1)[C@@H](OCc1ccccc1)[C@H](OCc1ccccc1)[C@@H](OCc1ccccc1)C(=O)N1CCN(C(c2ccccc2)(c2ccccc2)c2ccccc2)CC1 ; Cc1ccc(S(=O)(=O)N2CCN(C(=O)[C@H](OCc3ccccc3)[C@@H](OCc3ccccc3)[C@H](OCc3ccccc3)[C@@](O)(COCc3ccccc3)[C@@H](C)O)CC2)cc1]

In still other embodiments, the present invention relates to a method for preparing compound (V-1) comprising: reacting compound (VI-a) with methylating reagent 1 through an addition reaction in the presence of isopropyl titanate to obtain compound (V-1);

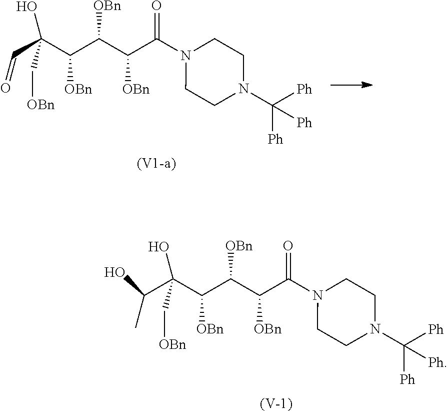 [SMILES: O=C[C@](O)(COCc1ccccc1)[C@@H](OCc1ccccc1)[C@H](OCc1ccccc1)[C@@H](OCc1ccccc1)C(=O)N1CCN(C(c2ccccc2)(c2ccccc2)c2ccccc2)CC1 ; "(V1-a)" ; C[C@@H](O)[C@](O)(COCc1ccccc1)[C@@H](OCc1ccccc1)[C@H](OCc1ccccc1)[C@@H](OCc1ccccc1)C(=O)N1CCN(C(c2ccccc2)(c2ccccc2)c2ccccc2)CC1 ; "(V-1)"]

Preferably, the compound (VI-a) can be prepared according to the preparation method of compound (VIa) of the present invention.

Preferably, the method for preparing compound (V-1) can be referenced to the preparation method of compound (Va) of the present invention.

In still other embodiments, the present invention relates to a method for preparing compound (V-2) comprising: reacting compound (Vc) with p-toluenesulfonyl chloride to obtain compound (V-2);

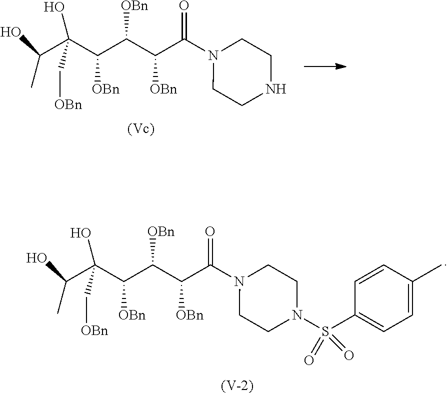 [SMILES: C[C@@H](O)[C@](O)(COCc1ccccc1)[C@@H](OCc1ccccc1)[C@H](OCc1ccccc1)[C@@H](OCc1ccccc1)C(=O)N1CCNCC1 ; Cc1ccc(S(=O)(=O)N2CCN(C(=O)[C@H](OCc3ccccc3)[C@@H](OCc3ccccc3)[C@H](OCc3ccccc3)[C@@](O)(COCc3ccccc3)[C@@H](C)O)CC2)cc1 ; "(V-2)"]

Preferably, the compound (Vc) can be prepared by the method described herein.

Preferably, the reaction between compound (Vc) and p-toluenesulfonyl chloride is carried out in the presence of a base; wherein the base includes but is not limited to triethylamine, pyridine, DMAP and the like.

In other embodiments, the compound (III) can be prepared by the following method B:

Method B:

Step 1: reacting compound (Vc) with acid 1 to obtain compound (Vd),

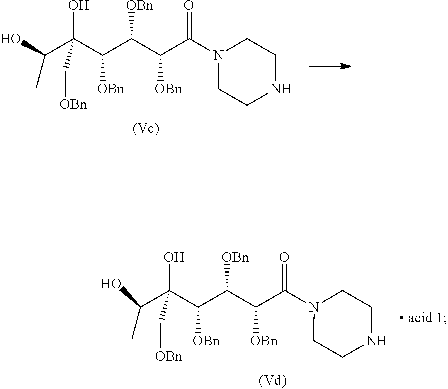 [SMILES: C[C@@H](O)[C@](O)(COCc1ccccc1)[C@@H](OCc1ccccc1)[C@H](OCc1ccccc1)[C@@H](OCc1ccccc1)C(=O)N1CCNCC1 ; C[C@@H](O)[C@](O)(COCc1ccccc1)[C@@H](OCc1ccccc1)[C@H](OCc1ccccc1)[C@@H](OCc1ccccc1)C(=O)N1CCNCC1]

Step 2: reacting compound (Vd) with hydroxyl protecting reagent 2 to obtain compound (Ve),

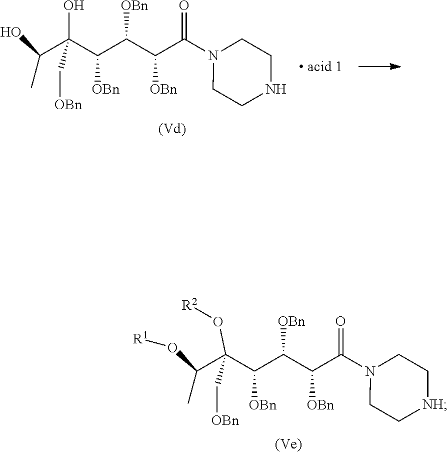

Step 3: reacting compound (Ve) in the presence of acid 2 to obtain compound (Vf),

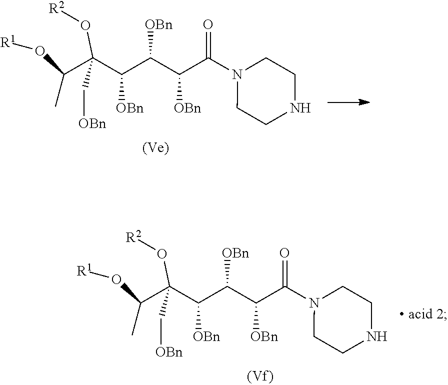

Step 4: reacting compound (Vf) with compound R³X¹ to obtain compound (III),

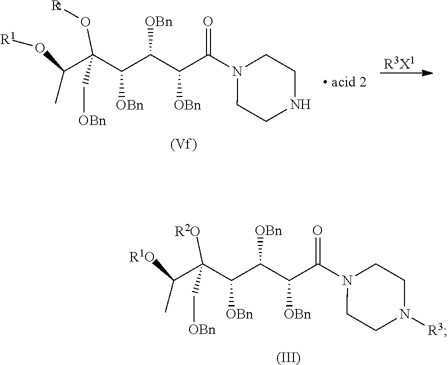

wherein, X¹ is Cl, Br or I;

each acid 1 and acid 2 is independently sulfuric acid, hydrochloric acid, nitric acid, phosphoric acid, oxalic acid, pivalic acid, methanesulfonic acid, acetic acid, formic acid, benzoic acid, p-toluenesulfonic acid, citric acid, cinnamic acid, tartaric acid, malic acid, salicylic acid, succinic acid or caffeic acid; and

each R¹, R² and R³ is independently as described herein.

Preferably, the acid 1 is sulfuric acid, methanesulfonic acid or oxalic acid.

In other embodiments, the compound (III) provided herein has one of the following structures:

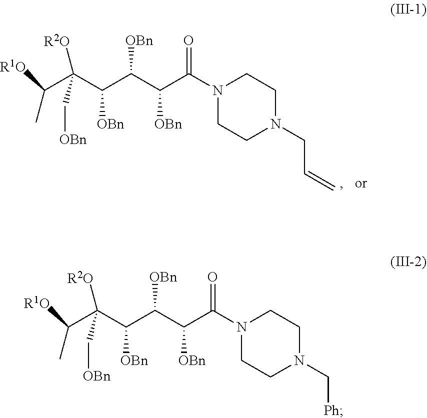

Wherein, each R¹ and R² is independently as described in the present invention.

Preferably, the compounds (III-1) and (III-2) can each independently be prepared by the method B provided herein.

In still other embodiments, when R¹, R² together with —OCHCHO— to which they are attached form

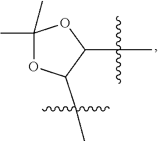 [SMILES: *C1OC(C)(C)OC1*]

each of the compounds (III-1) and (III-2) also can independently be prepared by the method B1 or B2 provided herein.

In other embodiments, the compound (Vc) can be prepared by the method provided herein. Preferably, the compound (Vc) can be prepared by the method M provided herein.

In other embodiments, the compound (Vc) can be prepared by the following method:

removing the tert-butoxycarbonyl protecting group from compound (Vb) to obtain compound (Vc),

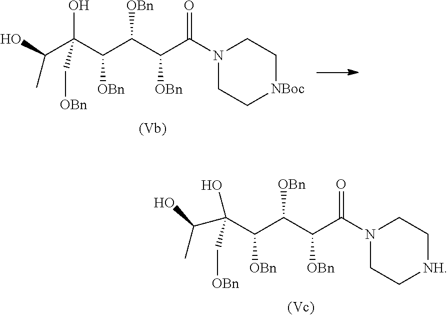 [SMILES: C[C@@H](O)[C@](O)(COCc1ccccc1)[C@@H](OCc1ccccc1)[C@H](OCc1ccccc1)[C@@H](OCc1ccccc1)C(=O)N1CCN(C(=O)OC(C)(C)C)CC1 ; C[C@@H](O)[C@](O)(COCc1ccccc1)[C@@H](OCc1ccccc1)[C@H](OCc1ccccc1)[C@@H](OCc1ccccc1)C(=O)N1CCNCC1]

In still other embodiments, the compound (Vb) can be prepared according to the preparation method of compound (Va) provided herein.

In still other embodiments, the reaction condition for removing the tert-butoxycarbonyl group comprises: increasing the temperature of the addition reaction to 30° C. 50° C. on the basis of the addition reaction of the compound (Va); preferably, increasing the temperature of the addition reaction to 30° C.˜40° C.; preferably, increasing the temperature of the addition reaction to 30° C.˜35° C.

In other embodiments, in method B, the acid 1 is used to adjust the pH of the solution; wherein after acid 1 is added, the pH of the solution is 1.0˜7.0; preferably, after acid 1 is added, the pH of the solution is 3.0˜7.0; preferably, after acid 1 is added, the pH of the solution is 5.5˜7.0.

In other embodiments, in method B, the hydroxyl protecting reagent 2 is 2,2-dimethoxypropane, benzaldehyde dimethyl acetal, trimethylchlorosilane or tert-butyldimethylchlorosilane.

In other embodiments, the reaction solvent in step 2 of the method B is toluene, 1,2-dichloroethane, dichloromethane, methyl tert-butyl ether, isopropyl ether, tetrahydrofuran, methyltetrahydrofuran (e.g., 2-methyltetrahydrofuran) or any combination thereof.

In other embodiments, the reaction temperature in step 2 of the method B is 0° C.˜40° C.; preferably, the reaction temperature in step 2 of the method B is 10° C.˜40° C.; preferably, the reaction temperature in step 2 of the method B is 8° C.˜30° C.

In other embodiments, the reaction of step 2 of the method B is optionally carried out in the presence of methanesulfonic acid.

In other embodiments, in method B, the amount of substance of acid 2 is 0.5 to 2.0 times that of compound (Ve); preferably, the amount of substance of acid 2 is 0.5 to 1.2 times that of compound (Ve).

In one aspect, the present invention relates to a compound having one of the following structures or a salt thereof:

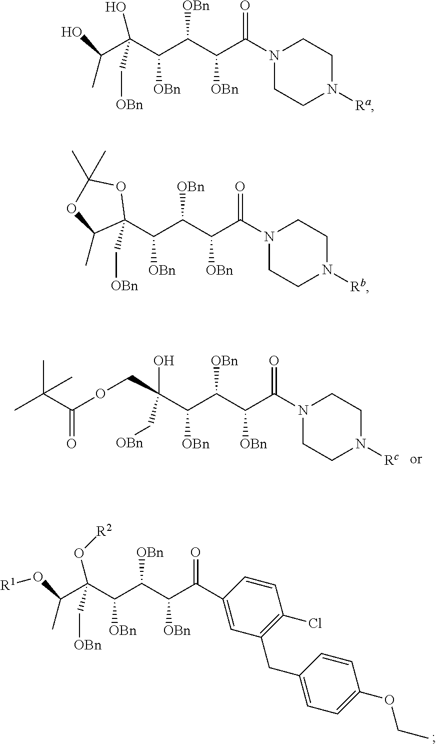

wherein, each R^{a}, R^{b} and R^{c} is independently H, C₂₋₈ alkyl, C₄₋₈ alkenyl, allyl, phenyl, benzyl, p-toluenesulfonyl, benzenesulfonyl, 4-bromobenzenesulfonyl, 4-nitrophenyl, 1,3-dichlorophenyl, tert-Butoxycarbonyl, triphenylmethyl, bis(4-methoxyphenyl)(phenyl)methyl, diphenylmethyl, N,N-diphenylaminoacyl, pyridyl, benzylsulfonyl, imidazolyl, N,N-dimethylaminosulfonyl, N,N-dimethylaminoacyl or

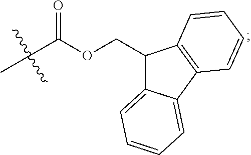 [SMILES: *C(=O)OCC1c2ccccc2-c2ccccc21]

each R¹ and R² is independently benzyl, triphenylmethyl, p-methoxybenzyl, tert-butyldimethylsilyl, trimethylsilyl, tert-butyldiphenylsilyl, triethyl silyl, triisopropylsilyl, benzyloxycarbonyl, 2-(trimethylsilyl)ethoxymethyl, dihydropyranyl, bromopropenyl, ethylformyl, acetyl or benzoyl, or, R¹ and R² together with —OCHCHO— to which they are attached form

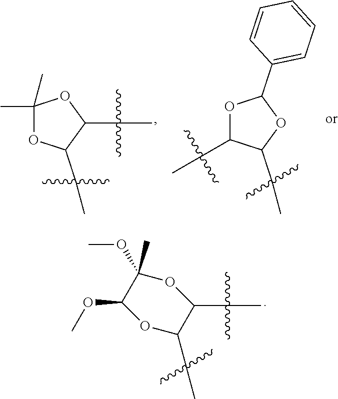

In some embodiments, the salt is methanesulfonate or oxalate.

In some embodiments, the present invention relates to a compound having one of the following structures:

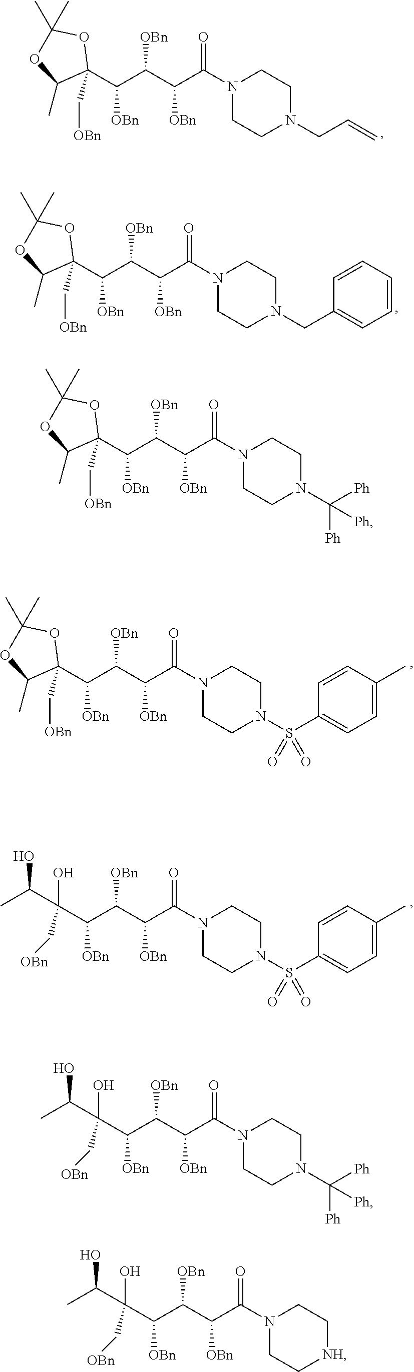

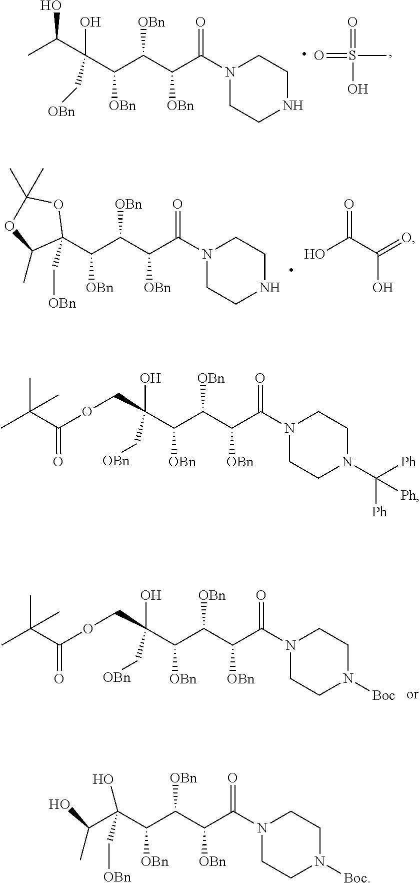

Specifically, each of the above-mentioned compounds independently can be used as an intermediate compound to prepare the compound (I).

In other aspect, the present invention relates to a method for preparing compound (I) comprising the steps of:

Step (A-1): reacting compound (IIIa) or a salt thereof with compound (IV) through a coupling reaction to obtain compound (IIa),

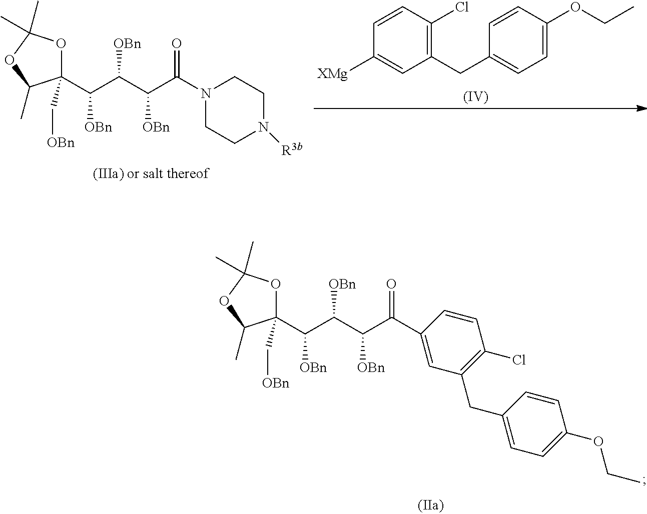

Step (B-1): reacting compound (IIa) through hydrogenation reduction and ring closure under acidic condition to obtain compound (I),

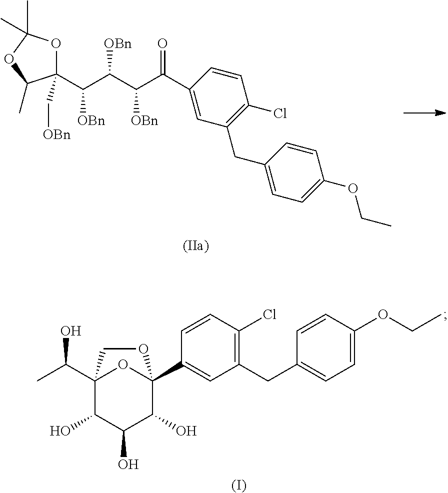

wherein,

X is Cl, Br or I;

R^{3b} is benzyl, C₂₋₈ alkyl, C₄₋₈ alkenyl, allyl, phenyl, p-toluenesulfonyl, benzenesulfonyl, 4-bromobenzenesulfonyl, 4-nitrophenyl, 1,3-dichlorophenyl, triphenylmethyl, bis(4-methoxyphenyl)(phenyl)methyl, diphenylmethyl, N,N-diphenylaminoacyl, pyridyl, benzylsulfonyl, imidazolyl, N,N-dimethylaminosulfonyl, N,N-dimethylaminoacyl or

In some embodiments, the compound (IIIa) is prepared by the following method B1, method B1:

Step 1: reacting compound (Vc) with acid 1 to obtain compound (Vd),

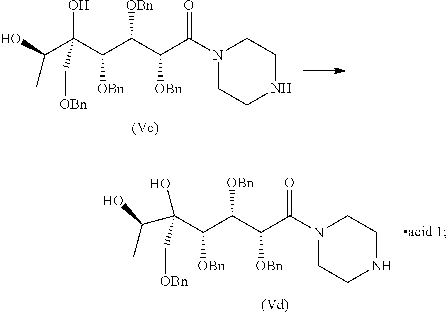 [SMILES: C[C@@H](O)[C@](O)(COCc1ccccc1)[C@@H](OCc1ccccc1)[C@H](OCc1ccccc1)[C@@H](OCc1ccccc1)C(=O)N1CCNCC1 ; C[C@@H](O)[C@](O)(COCc1ccccc1)[C@@H](OCc1ccccc1)[C@H](OCc1ccccc1)[C@@H](OCc1ccccc1)C(=O)N1CCNCC1]

Step 2: reacting compound (Vd) with 2,2-dimethoxypropane to obtain compound (Vea),

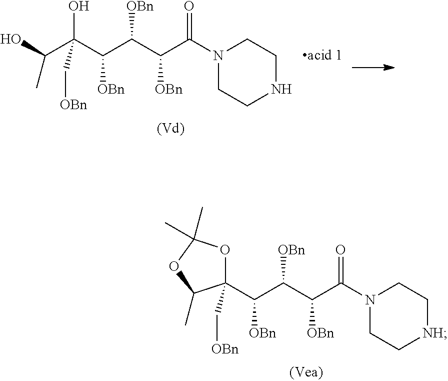 [SMILES: C[C@@H](O)[C@](O)(COCc1ccccc1)[C@@H](OCc1ccccc1)[C@H](OCc1ccccc1)[C@@H](OCc1ccccc1)C(=O)N1CCNCC1 ; C[C@H]1OC(C)(C)O[C@@]1(COCc1ccccc1)[C@@H](OCc1ccccc1)[C@H](OCc1ccccc1)[C@@H](OCc1ccccc1)C(=O)N1CCNCC1]

Step 3: reacting compound (Vea) in the presence of acid 2 to obtain compound (Vfa),

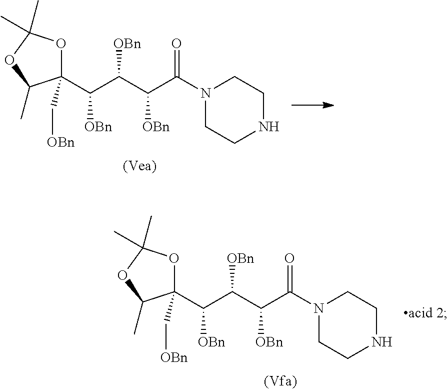 [SMILES: C[C@H]1OC(C)(C)O[C@@]1(COCc1ccccc1)[C@@H](OCc1ccccc1)[C@H](OCc1ccccc1)[C@@H](OCc1ccccc1)C(=O)N1CCNCC1 ; C[C@H]1OC(C)(C)O[C@@]1(COCc1ccccc1)[C@@H](OCc1ccccc1)[C@H](OCc1ccccc1)[C@@H](OCc1ccccc1)C(=O)N1CCNCC1]

Step 4: reacting compound (Vfa) with compound R^{3b}X^{1a} to obtain compound (IIIa),

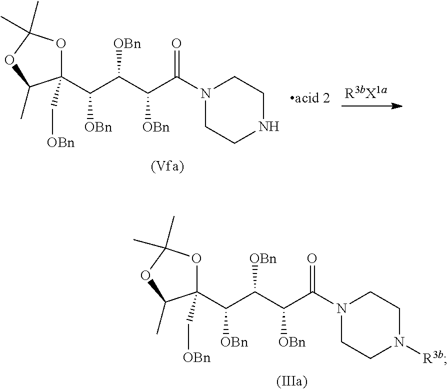

wherein, X^{1a} is Cl, Br or I;

each acid 1 and acid 2 is independently sulfuric acid, hydrochloric acid, nitric acid, phosphoric acid, oxalic acid, pivalic acid, methanesulfonic acid, acetic acid, formic acid, benzoic acid, p-toluenesulfonic acid, citric acid, cinnamic acid, tartaric acid, malic acid, salicylic acid, succinic acid or caffeic acid;

R^{3b} is as described herein.

In other embodiments, the compound (Vc) is prepared by the preparation method provided herein.

In some embodiments, the compound (IIIa) is prepared by the following method B2, method B2:

Step 1: reacting compound (VIb) with methylating reagent 1 through an addition reaction in the presence of isopropyl titanate to obtain compound (Vb),

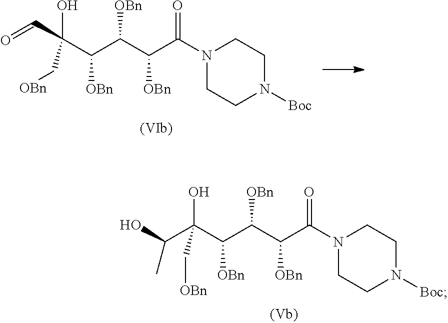 [SMILES: CC(C)(C)OC(=O)N1CCN(C(=O)[C@H](OCc2ccccc2)[C@@H](OCc2ccccc2)[C@H](OCc2ccccc2)[C@](O)(C=O)COCc2ccccc2)CC1 ; C[C@@H](O)[C@](O)(COCc1ccccc1)[C@@H](OCc1ccccc1)[C@H](OCc1ccccc1)[C@@H](OCc1ccccc1)C(=O)N1CCN(C(=O)OC(C)(C)C)CC1]

Step 2: removing the tert-butoxycarbonyl protecting group from compound (Vb) to obtain compound (Vc),

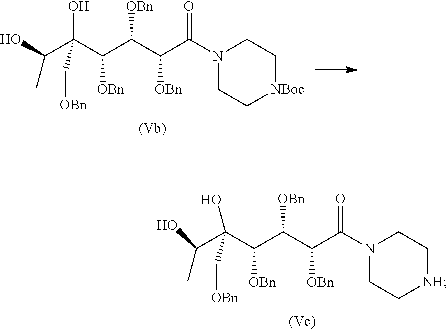 [SMILES: C[C@@H](O)[C@](O)(COCc1ccccc1)[C@@H](OCc1ccccc1)[C@H](OCc1ccccc1)[C@@H](OCc1ccccc1)C(=O)N1CCN(C(=O)OC(C)(C)C)CC1 ; C[C@@H](O)[C@](O)(COCc1ccccc1)[C@@H](OCc1ccccc1)[C@H](OCc1ccccc1)[C@@H](OCc1ccccc1)C(=O)N1CCNCC1]

Step 3: reacting compound (Vc) with acid 1 to obtain compound (Vd),

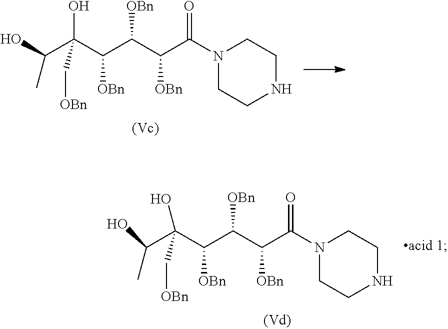 [SMILES: C[C@@H](O)[C@](O)(COCc1ccccc1)[C@@H](OCc1ccccc1)[C@H](OCc1ccccc1)[C@@H](OCc1ccccc1)C(=O)N1CCNCC1 ; C[C@@H](O)[C@](O)(COCc1ccccc1)[C@@H](OCc1ccccc1)[C@H](OCc1ccccc1)[C@@H](OCc1ccccc1)C(=O)N1CCNCC1]

Step 4: reacting compound (Vd) with 2,2-dimethoxypropane to obtain compound (Vea),

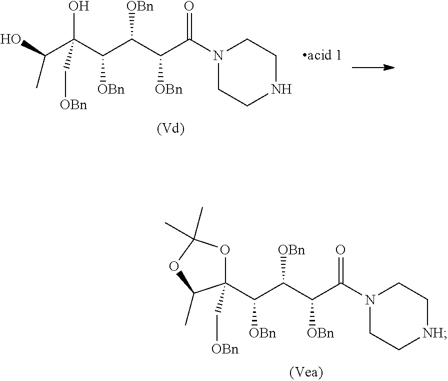 [SMILES: C[C@@H](O)[C@](O)(COCc1ccccc1)[C@@H](OCc1ccccc1)[C@H](OCc1ccccc1)[C@@H](OCc1ccccc1)C(=O)N1CCNCC1 ; C[C@H]1OC(C)(C)O[C@@]1(COCc1ccccc1)[C@@H](OCc1ccccc1)[C@H](OCc1ccccc1)[C@@H](OCc1ccccc1)C(=O)N1CCNCC1]

Step 5: reacting compound (Vea) in the presence of acid 2 to obtain compound (Vfa),

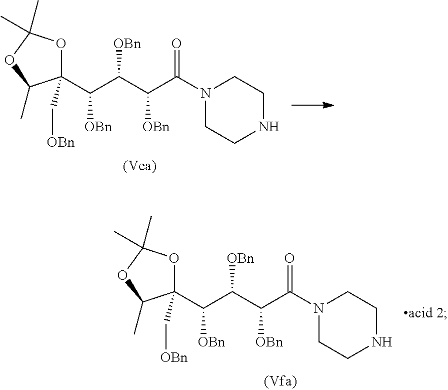 [SMILES: C[C@H]1OC(C)(C)O[C@@]1(COCc1ccccc1)[C@@H](OCc1ccccc1)[C@H](OCc1ccccc1)[C@@H](OCc1ccccc1)C(=O)N1CCNCC1 ; C[C@H]1OC(C)(C)O[C@@]1(COCc1ccccc1)[C@@H](OCc1ccccc1)[C@H](OCc1ccccc1)[C@@H](OCc1ccccc1)C(=O)N1CCNCC1]

Step 6: reacting compound (Vfa) with compound R^{3b}X^{1a} to obtain compound (IIIa),

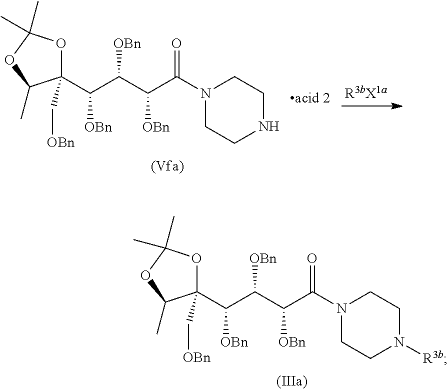

wherein, X^{1a} is Cl, Br or I;

each acid 1 and acid 2 is independently sulfuric acid, hydrochloric acid, nitric acid, phosphoric acid, oxalic acid, pivalic acid, methanesulfonic acid, acetic acid, formic acid, benzoic acid, p-toluenesulfonic acid, citric acid, cinnamic acid, tartaric acid, malic acid, salicylic acid, succinic acid or caffeic acid; and

R^{3b} is as described herein.

In some embodiments, the acid 1 in Step 1 of method B1 and Step 3 of method B2 are independently preferably sulfuric acid, methanesulfonic acid or oxalic acid.

In some embodiments, the acidic condition in step (B-1) refers to the reaction reacts in the presence of an acid, and the acid is hydrochloric acid or acetic acid; the hydrogenation reduction reaction is carried out in the presence of a catalyst, and the catalyst is palladium/carbon, palladium hydroxide/carbon, platinum/carbon, Raney nickel or palladium chloride, wherein, the mass ratio of the catalyst and compound (IIa) is (0.02:1)˜(0.8:1); preferably, the mass ratio of the catalyst and compound (IIa) is (0.1:1)˜(0.6:1); preferably, the mass ratio of the catalyst and compound (IIa) is (0.05:1)˜(0.2:1).

In some embodiments, in step (A-1), the amount of substance of compound (IV) is 1.0 to 1.5 times that of compound (TITa) or a salt thereof; preferably, in step (A-1), the amount of substance of compound (IV) is 1.1 to 1.4 times that of compound (IIIa) or a salt thereof; preferably, in step (A-1), the amount of substance of compound (IV) is 1.2 to 1.4 times that of compound (IIIa) or a salt thereof; preferably, in step (A-1), the amount of substance of compound (IV) is 1.1 to 1.3 times that of compound (IIIa) or a salt thereof;

In some embodiments, the reaction solvent in step (A-1) is tetrahydrofuran, methyltetrahydrofuran, methyl tert-butyl ether, toluene, dichloromethane or any combination thereof.

In some embodiments, the reaction temperature in step (A-1) is 10° C.˜40° C.; preferably, the reaction temperature in step (A-1) is 20° C.˜30° C.

In some embodiments, the acid 1 in step 1 of the method B1 and step 3 of the method B2 are independently used to adjust the pH of the solution; specifically, after acid 1 is added, the pH of the solution is 3.0˜7.0; preferably, after acid 1 is added, the pH of the solution is 5.5˜7.0.

In some embodiments, the reaction solvents in step 1 of the method B1 and step 3 of the method B2 are each independently toluene, 1,2-dichloroethane, dichloroethane, methyl tert-butyl ether, isopropyl ether, tetrahydrofuran, 2-methyltetrahydrofuran or any combination thereof.

In some embodiments, the reaction temperature in step 1 of the method B1 and step 3 of the method B2 are each independently room temperature; preferably, the reaction temperature in step 1 of the method B1 and step 3 of the method B2 are each independently 8° C.˜30° C.

In some embodiments, the reactions in step 1 of the method B1 and step 3 of the method B2 are each independently carried out in the presence of methanesulfonic acid.

In some embodiments, in step 3 of method B1 or step 5 of method B2, the amount of substance of acid 2 is 0.5 to 2.0 times that of compound (Vea); preferably, the amount of substance of acid 2 is 0.5 to 1.2 times that of compound (Vea).

In other embodiments, in step 1 of the method B2, the reaction is optionally carried out in the presence of a chiral ligand 1; wherein, the chiral ligand 1 is a dihydroxy chiral ligand or a metal ligand.

In still other embodiments, in step 1 of the method B2, the dihydroxy chiral ligand is R-1,1′-Bi-2-naphthol ((R)-BINOL), (4R,5R)-2,2-dimethyl-a,a,a′,a′-tetraphenyl-1,3-dioxolane-4,5-dimethanol (TADDOL), (S)-(−)-5,5′,6,6′,7,7′,8,8′-octahydro-1,1′-2-naphthol ((S)-H₈-BINOL) or Salen ligand.

In still other embodiments, in step 1 of the method B2, the metal ligand is metal-Salen ligand, metal-BINOL ligand or (1R,2R)-(+)-N,N′-Di-p-toluenesulfonyl-1,2-cyclohexanediamine-metal ligand.

In still other embodiments, the metal-Salen ligand in step 1 of the method B2 is Zn-Salen ligand, Mn-Salen ligand, Ti-Salen ligand or Cr-Salen ligand; preferably, the metal-Salen ligand is Ti-Salen ligand.

In still other embodiments, the metal-BINOL ligand in step 1 of the method B2 is Zn-BINOL ligand, Mn-BINOL ligand, Ti-BINOL ligand or Cr-BINOL ligand.

In still other embodiments, BINOL of the metal-BINOL ligand in step 1 of the method B2 is R configuration or S configuration.

In still other embodiments, in step 1 of the method B2, the Salen ligand, Zn-Salen ligand, Mn-Salen ligand, Ti-Salen ligand and Cr-Salen ligand are respectively preferably selected from the following structures:

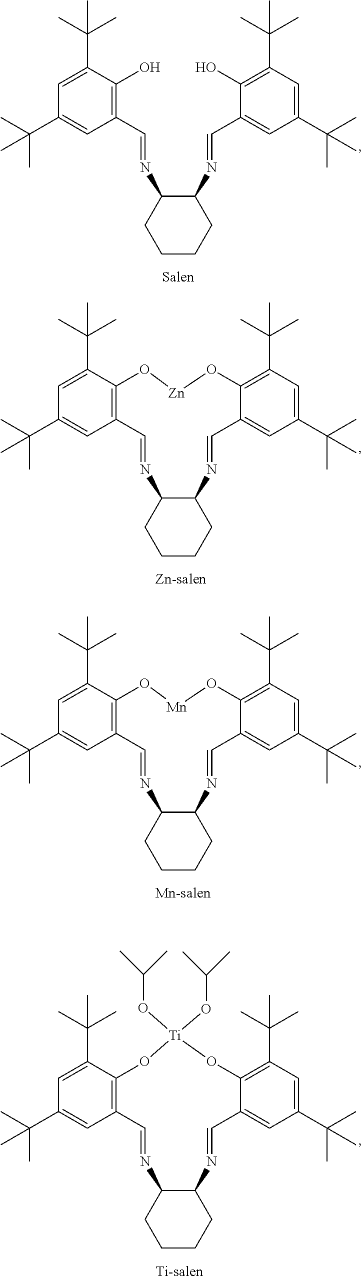

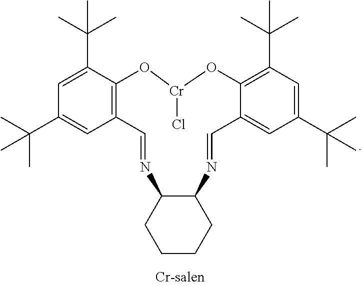

In still other embodiments, in step 1 of the method B2, the amount of substance of metal-Salen ligand is 0.1 to 1.0 time that of compound (VIb).

In still other embodiments, in step 1 of the method B2, the amount of substance of Salen ligand is 0.10 to 1.0 time that of compound (VIb).

In still other embodiments, in step 1 of the method B2, the amount of substance of R-1,1′-Bi-2-naphthol is 0.01 to 0.9 times that of compound (VIb); preferably, the amount of substance of R-1,1′-Bi-2-naphthol is 0.05 to 0.2 times that of compound (VIb); preferably, the amount of substance of R-1,1′-Bi-2-naphthol is 0.05, 0.1, or 0.2 times that of compound (VIb).

In still other embodiments, in step 1 of the method B2, the amount of substance of Ti-BINOL ligand is 0.01 to 0.20 times that of compound (VIb); preferably, the amount of substance of Ti-BINOL ligand is 0.05 to 0.20 times that of compound (VIb).

In other embodiments, in step 1 of the method B2, the amount of substance of isopropyl titanate is 0.5 to 8.0 times that of compound (VIb); preferably, the amount of substance of isopropyl titanate is 1.0 to 5.0 times that of compound (VIb); preferably, the amount of substance of isopropyl titanate is 1.4 to 4.0 times that of compound (VIb); preferably, the amount of substance of isopropyl titanate is 2.0 to 4.0 times that of compound (VIb); preferably, the amount of substance of isopropyl titanate is 3.9 times that of compound (VIb); preferably, the amount of substance of isopropyl titanate is 1.4, 2, 3 or 4 times that of compound (VIb).

Optionally, in step 1 of the method B2, the tetraisopropyl titanate can be replaced with CrCl₂, ZnCl₂, MnCl₂ or cobalt acetate.

In other embodiments, in step 1 of the method B2, the methylating reagent 1 ismethylmagnesium bromide, methylmagnesium chloride, methyllithium, trimethylaluminum or dimethyl zinc.

In still other embodiments, in step 1 of the method B2, the amount of substance of methylmagnesium bromide is 3.0 to 6.0 times that of compound (VIb); preferably, the amount of substance of methylmagnesium bromide is 4.0 to 6.0 times that of compound (VIb); preferably, the amount of substance of methylmagnesium bromide is 4.0 to 5.0 times that of compound (VIb); preferably, the amount of substance of methylmagnesium bromide is 4.0, 5.0 or 6.0 times that of compound (VIb).

As described in the present invention, the methylmagnesium bromide can be a solution of methylmagnesium bromide in 2-methyltetrahydrofuran with a concentration of 3 mol/L.

In other embodiments, in step 1 of the method B2, the reaction solvent is dichloromethane, tetrahydrofuran, methyl tert-butyl ether, toluene, o-xylene, p-xylene, meta-xylene or any combination thereof. Preferably, in step 1 of the method B2, the reaction solvent is dichloromethane.

In other embodiments, in step 1 of the method B2, the reaction temperature is 10° C.˜40° C.; preferably, the reaction temperature is 20° C.˜35° C.; preferably, the reaction temperature is 20° C.˜32° C.; preferably, the reaction temperature is 20° C.˜30° C.

In other embodiments, in step 1 of the method B2, compound (VIb) is added by dropping; optionally, the reaction system is kept at a certain temperature. Preferably, the temperature of the reaction system during the dropping is −20° C.˜25° C.; preferably, the temperature of the reaction system during the dropping is −10° C.˜0° C.; preferably, the temperature of the reaction system during the dropping is −5° C.˜0° C.

In other embodiments, in step 2 of the method B2, the reaction condition for removing the tert-butoxycarbonyl group comprises: increasing the temperature of the addition reaction to 30° C.˜50° C. on the basis of the addition reaction to prepare compound (Va); preferably, increasing the temperature of the addition reaction to 30° C.˜40° C.; preferably, increasing the temperature of the addition reaction to 30° C.˜35° C.

In other embodiments, in step 2 of the method B2, the reaction for removing the tert-butoxycarbonyl group can be carried out in the presence of an acid; wherein the acid can be hydrochloric acid, trifluoroacetic acid, or the like.

As described herein, in step 1 of method B2, a new chiral center can be introduced through an asymmetric addition reaction of an aldehyde group with methylating reagent, and a product with high ee value and intermediate (Vb) with a high yield can be obtained through selection and optimization of methylating reagents, chiral ligands and/or other conditions. Specifically, the type and dosage of methylating reagent have different effects on the reaction. When methylating reagent is methylmagnesium bromide, and the amount of methylmagnesium bromide is 4.0 to 6.0 equivalents, the reaction is complete and the ee value of the product is high.

As described herein, different hydroxyl protecting groups have different effects on the reaction. After a large number of screening tests, it is found that when the hydroxyl protecting group is 2,2-dimethoxypropane or benzaldehyde dimethyl acetal, the yield of the reaction is high, and the obtained compound (III) or (Ve) is more stable and has high purity.

Optionally, the hydrogen source of the hydrogenation reduction reaction is hydrogen.

In some embodiments, the compound (VIb) can be prepared according to the preparation method of the compound (VIa) of the present invention.

Optionally, the compound (VIb) can be prepared by the following method: Step a-1: reacting compound (VIIIb) in the presence of alkaline reagent 1 through a hydrolysis reaction to obtain compound (VIIb),

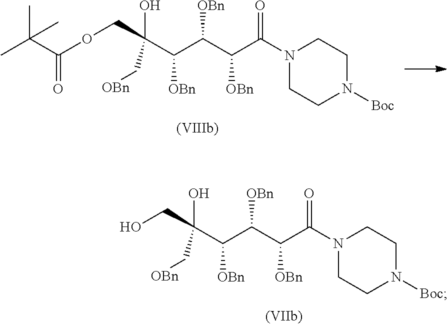 [SMILES: CC(C)(C)OC(=O)N1CCN(C(=O)[C@H](OCc2ccccc2)[C@@H](OCc2ccccc2)[C@H](OCc2ccccc2)[C@@](O)(COCc2ccccc2)COC(=O)C(C)(C)C)CC1 ; CC(C)(C)OC(=O)N1CCN(C(=O)[C@H](OCc2ccccc2)[C@@H](OCc2ccccc2)[C@H](OCc2ccccc2)[C@](O)(CO)COCc2ccccc2)CC1]

Step b-1: reacting compound (VIIb) in the presence of oxidant 1 through an oxidation reaction to obtain compound (VIb),

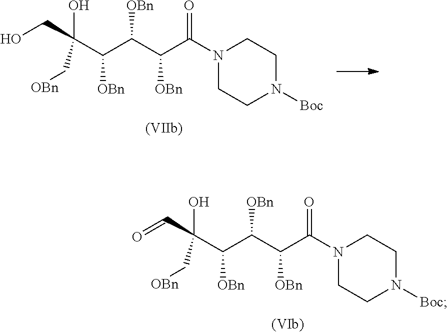

wherein, the alkaline reagent 1 and the oxidant 1 are each independently as described herein.

Optionally, the reaction conditions of step a-1 are the same or similar to the reaction conditions of step a of the present invention.

Optionally, the reaction conditions of step b-1 are the same or similar to the reaction conditions of step b of the present invention.

Optionally, the compound (VIIIb) can be further purified according to the purification method of compound (VIIIa).

Optionally, the compound (VIb) can be further purified according to the purification method of compound (VIa).

The compound (VIIIa) or (VIIIb) can be further purified and then used to prepare the compound (VIa) or (VIb). Wherein, the recrystallization step in the purification process can be repeated multiple times, and the solvent used for each recrystallization can be the same or different to achieve a better purification effect. For example, by successively using a mixed solvent of methyl tert-butyl ether and n-heptane, a mixed solvent of acetone and water, a mixed solvent of ethyl acetate and n-heptane for recrystallization purification, a high-purity (up to 99.5%) compound (VIIIa) or (VIIIb) can be obtained.

Recrystallization of compound (VIIIa) or (VIIIb) can effectively remove the impurities (e.g. iodomethyl pivalate) after the Grignard reaction for preparing the compound (VIIIa) or (VIIIb), so that it is easier to trace impurities in the reaction; and the purification method is simple, controllable, and it is conducive to the control of the next reaction step; also, compound (VIIIa) or (VIIIb) obtained through purification can give compound (VIa) or (VIb) with high yield and purity through a hydrolysis reaction and an oxidation reaction. After a lot of experiments, the inventors found that when the compound (VIIIa) or (VIIIb) is not purified, the total yield of the compound (I) is low; at the same time, intermediate (II) or (IIa) needs to be purified by silica gel column chromatography in work-up, which is not conducive to industrial production.

Optionally, the compound (VIIIb) provided herein may be a single crystal form, an amorphous form or a mixture of multiple crystal forms. Optionally, the compound (VIIIb) provided herein is a single crystal form. The purity of the single crystal form is high, which is beneficial to the subsequent reaction, including impurity control and traceability, etc., and the target product can be obtained with high yield and high purity.

Preferably, the compound (VIIIb) provided herein is crystal form A, and the X-ray powder diffraction pattern of the crystal form A has diffraction peaks at the following 20 angles: 6.52°±0.2°, 6.89°±0.2°, 9.25°±0.2°, 9.66°±0.2°, 13.72°±0.2°, 14.44°±0.2°, 14.97°±0.2°, 15.30°±0.2°, 16.18°±0.2°, 16.95°±0.2°, 17.19°±0.2°, 18.21°±0.2°, 18.44°±0.2°, 18.56°±0.2°, 18.86°±0.2°, 19.33°±0.2°, 19.56°±0.2°, 20.13°±0.2°, 20.53°±0.2°, 20.98°±0.2°, 21.39°±0.2°, 21.87°±0.2°, 22.38°±0.2°, 22.67°±0.2°, 23.12°±0.2°, 23.52°±0.2, 23.95°±0.2°, 24.38°±0.2°, 24.88°±0.2°, 26.07°±0.2°, 27.58°±0.2°, 29.11°±0.2°, 30.54°±0.2°, 31.07°±0.2°, 31.74°±0.2°, 32.76°±0.2°, 34.69°±0.2°, 35.06°±0.2°, 39.58°±0.2°, 39.89°±0.2°, 40.51°±0.2°.

Specifically, the compound (VIIIb) provided herein is crystal form A which has an X-ray powder diffraction pattern substantially as shown in FIG. 1.

On the other hand, the present invention relates to the crystal form A of the compound (VIIIb),

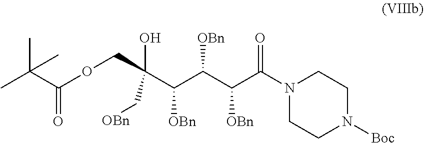 [SMILES: CC(C)(C)OC(=O)N1CCN(C(=O)[C@H](OCc2ccccc2)[C@@H](OCc2ccccc2)[C@H](OCc2ccccc2)[C@@](O)(COCc2ccccc2)COC(=O)C(C)(C)C)CC1]

wherein the X-ray powder diffraction pattern of the crystal form A has diffraction peaks at the following 2θ angles: 6.52°±0.2°, 6.89°±0.2°, 9.25°±0.2°, 9.66°±0.2°, 16.18°±0.2°, 18.44°±0.2°, 18.56°±0.2°, 21.87°±0.2°, 27.58°±0.2°.

In some embodiments, the compound (VIIIb) provided herein is crystal form A, and the X-ray powder diffraction pattern of the crystal form A has diffraction peaks at the following 2θ angles: 6.52°±0.2°, 6.89°±0.2°, 9.25°±0.2°, 9.66°±0.2°, 13.72°±0.2°, 16.18°±0.2°, 18.44°±0.2°, 18.56°±0.2°, 18.86°±0.2°, 19.33°±0.2°, 20.53°±0.2°, 21.39°±0.2°, 21.87°±0.2°, 23.52°±0.2, 23.95°±0.2°, 24.38°±0.2°, 26.07°±0.2°, 27.58°±0.2°.

In some embodiments, the compound (VIIIb) provided herein is crystal form A, and the X-ray powder diffraction pattern of the crystal form A has diffraction peaks at the following 2θ angles: 6.52°±0.2°, 6.89°±0.2°, 9.25°±0.2°, 9.66°±0.2°, 13.72°±0.2°, 14.44°±0.2°, 14.97°±0.2°, 15.30°±0.2°, 16.18°±0.2°, 16.95°±0.2°, 17.19°±0.2°, 18.21°±0.2°, 18.44°±0.2°, 18.56°±0.2°, 18.86°±0.2°, 19.33°±0.2°, 19.56°±0.2°, 20.13°±0.2°, 20.53°±0.2°, 20.98°±0.2°, 21.39°±0.2°, 21.87°±0.2°, 22.38°±0.2°, 22.67°±0.2°, 23.12°±0.2°, 23.52°±0.2, 23.95°±0.2°, 24.38°±0.2°, 24.88°±0.2°, 26.07°±0.2°, 27.58°±0.2°, 29.11°±0.2°, 30.54°±0.2°, 31.07°±0.2°, 31.74°±0.2°, 32.76°±0.2°, 34.69°±0.2°, 35.06°±0.2°, 39.58°±0.2°, 39.89°±0.2°, 40.51°±0.2°.

In some embodiments, the compound (VIIIb) provided herein is crystal form A which has an X-ray powder diffraction pattern substantially as shown in FIG. 1.

In one aspect, provided herein is a method for preparing compound (VIII), comprising the steps of:

reacting compound (IX) and the Grignard reagent obtained by the Grignard exchange of iodomethyl pivalate and isopropyl magnesium chloride lithium chloride through an addition reaction to obtain compound (VIII)

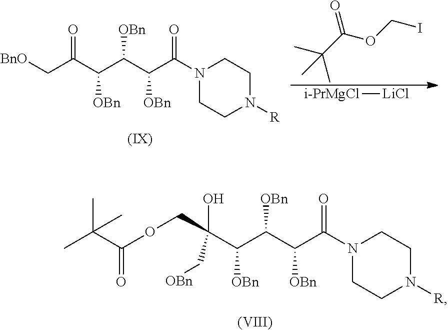

wherein, R is H, ethyl, n-propyl, n-butyl, tert-butyl, 2,2-dimethylpropyl, allyl, phenyl, benzyl, p-toluenesulfonyl, benzenesulfonyl, 4-bromobenzenesulfonyl, 4-nitrophenyl, 1,3-dichlorophenyl, tert-butoxycarbonyl, triphenylmethyl, bis(4-methoxyphenyl)benzyl, diphenylmethyl, N,N-diphenylaminoacyl, pyridyl, benzylsulfonyl, imidazolyl (e.g. imidazol-1-yl), N,N-dimethylaminosulfonyl, N,N-dimethylaminoacyl or

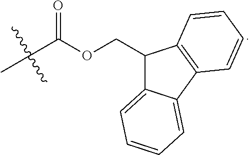 [SMILES: *C(=O)OCC1c2ccccc2-c2ccccc21]

In some embodiments, the method for preparing compound (VIII) comprises a purification method of compound (VIII) which comprises: adding the material containing compound (VIII) into solvent A, and stirring until clear/uniform, then adding solvent B to precipitate a solid compound (VIII). Specifically, the solvent A and the solvent B are each independently as described herein.

In some embodiments, the compound (IX) can be prepared by the following method:

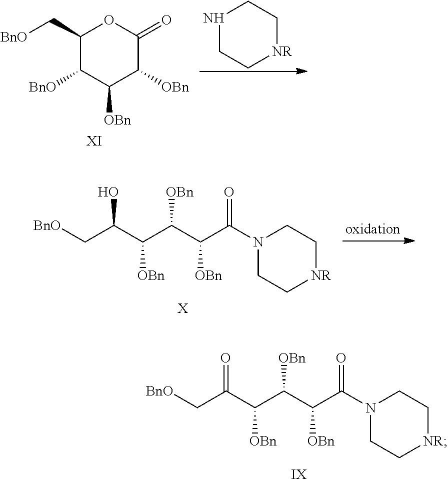

wherein R is as described herein.

On the other hand, provided herein is a compound with the structure shown in formula

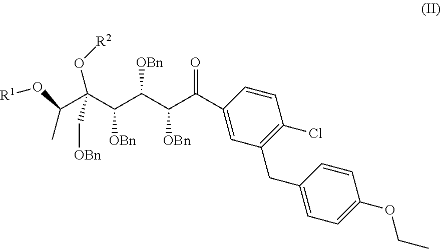

wherein, each R¹ and R² is independently benzyl, triphenylmethyl, p-methoxybenzyl, tert-butyldimethylsilyl, trimethylsilyl, tert-butyldiphenylsilyl, triethyl silyl, triisopropylsilyl, benzyloxycarbonyl, 2-(trimethylsilyl)ethoxymethyl, dihydropyranyl, bromopropenyl, ethylformyl, acetyl or benzoyl,

or, R¹ and R² together with —OCHCHO— to which they are attached form

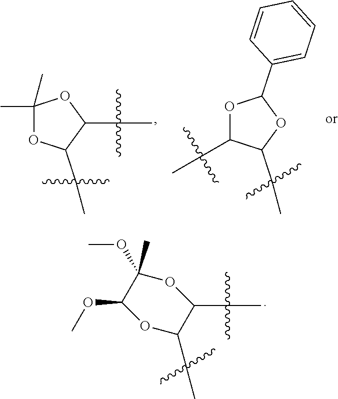

### DETAILED DESCRIPTION OF THE INVENTION

The present invention provides a method for preparing (1R,2S,3S,4R,5S)-5-[4-chloro-3-[(4-ethoxyphenyl)methyl]phenyl)-1-[(1R)-1-hydroxyethyl]-6,8-dio xbicyclo[3.2.1]octane-2,3,4-triol (I) used as a sodium-dependent glucose transporter (SGLT) inhibitor and a key intermediate thereof. Skilled in the art can learn from this invention to properly improve the process parameters to implement the preparation method. Of particular note is that all similar substitutions and modifications to the skilled person are obvious, and they are deemed to be included in the present invention.

### DEFINITIONS AND GENERAL TERMINOLOGY

Unless otherwise stated, the terms used in the specification and claims of the present invention have the following definitions.

Reference will now be made in detail to certain embodiments of the invention, examples of which are illustrated in the accompanying structures and formulas. The invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described herein. In the event that one or more of the incorporated literature, patents, and similar materials differs from or contradicts this application, including but not limited to defined terms, term usage, described techniques, or the like, this application controls.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Unless defined otherwise, all technical and scientific terms used herein have the same meaning as is commonly understood by one skilled in the art to which this invention belongs. All patents and publications referred to herein are incorporated by reference in their entirety.

As used herein, the following definitions shall apply unless otherwise indicated. For purposes of this invention, the chemical elements are identified in accordance with the Periodic Table of the Elements, CAS version, and the Handbook of Chemistry and Physics, 75th Ed. 1994. Additionally, general principles of organic chemistry are described in “Organic Chemistry”, Thomas Sorrell, University Science Books, Sausalito: 1999, and “March's Advanced Organic Chemistry” by Michael B. Smith and Jerry March, John Wiley & Sons, New York: 2007, the entire contents of which are hereby incorporated by reference.

The grammatical articles “a”, “an” and “the”, as used herein, are intended to include “at least one” or “one or more” unless otherwise indicated herein or clearly contradicted by the context. Thus, the articles used herein refer to one or more than one (i.e. at least one) articles of the grammatical objects. By way of example, “a component” means one or more components, and thus, possibly, more than one component is contemplated and may be employed or used in an implementation of the described embodiments.

The number of “equivalent” or “eq” refers to an equivalent amount of other needed material per 1 equivalent of the basic material in accordance with equivalent relation in chemical reaction.

The term “comprise” is an open expression, it means comprising the contents disclosed herein, but don't exclude other contents.

The term “room temperature” or “r.t.” refers to 10° C.˜ 40° C. In some embodiments, room temperature refers to 10° C.˜30° C. In still some embodiments, room temperature refers to 20° C.˜30° C.

Furthermore, what need to be explained is that the phrases “each . . . is independently” and “each of . . . and . . . is independently”, unless otherwise stated, should be broadly understood, which can mean that the specific options expressed by the same symbol are independent of each other in different groups; or the specific options expressed by the same symbol are independent of each other in same groups.

The term “protecting group” refers to a substituent that is commonly employed to block or protect a particular functionality while reacting with other functional groups on the compound. For example, an “amino-protecting group” is a substituent attached to an amino group that blocks or protects the amino functionality in the compound. Suitable amino-protecting groups include acetyl, trifluoroacetyl, t-butoxy-carbonyl (BOC, Boc), benzyloxycarbonyl (CBZ, Cbz) and 9-fluorenylmethylenoxy-carbonyl (Fmoc). Similarly, “hydroxy protecting group” is a substituent attached to a hydroxy group that blocks or protects the hydroxy functionality in the compound. Suitable hydroxy protecting groups include benzyl (Bn), carbobenzoxy (Cbz), triphenyl methyl, p-methoxybenzyl (PMB), t-butyldimethylsilyl (TBDMS), trimethylsilyl (TMS), t-butyldiphenylsilyl (TBDPS), triethylsilyl (TES), triisopropylsilyl (DIPS), 2-(trimethylsilyl)ethoxymethyl, dihydropyranyl, bromoallyl, ethoxycarbonyl, acetyl or benzoyl,

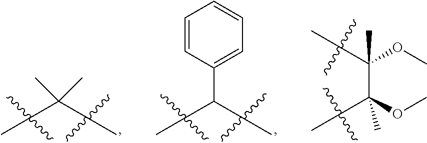

and the like. A “carboxy-protecting group” refers to a substituent of the carboxy group that blocks or protects the carboxy functionality. Common carboxy-protecting groups include —CH₂CH₂SO₂Ph, cyanoethyl, 2-(trimethylsilyl)ethyl, 2-(trimethylsilyl) ethoxy-methyl, 2-(p-toluenesulfonyl) ethyl, 2-(p-nitrophenylsulfonyl)-ethyl, 2-(diphenylphosphino)-ethyl, nitroethyl and the like. For a general description of protecting groups and their use, see T. W. Greene, Protective Groups in Organic Synthesis, John Wiley & Sons, New York, 1991; and P. J. Kocienski, Protecting Groups, Thieme, Stuttgart, 2005.

In context, all numbers disclosed herein are approximate values. Each numerical value may have a difference of 1%, 2%, 5%, 7%, 8% or 10%. When a number with an N value is made public, any number within N+/−1%, N+/−2%, N+/−3%, N+/−5%, N+/−7%, N+/−8%, or N+/−10% will be opened clearly, wherein “+/−” means plus or minus. Whenever a numerical range with a lower limit, DL, and an upper limit, DU, is disclosed, any number falling within the range is specifically disclosed.

After the reaction proceeds to a certain extent in the present invention, such as the raw material is consumed more than 70%, more than 80%, more than 90%, more than 95%, or completely by monitoring, the reaction mixture is worked up, such as cooled, collected, drawn, filtered, separated, purified or a combination thereof. The reaction can be monitored by conventional method such as thin-layer chromatography (TLC), high performance liquid chromatography (HPLC), gas chromatography (GC), and the like. The reaction mixture can be worked up by conventional method, for example, the crude product can be collected by concentrating the reaction mixture through vacuum evaporation or conventional distillation and is used directly in the next operation; or the crude product can be obtained by filtration of the reaction mixture and is used directly in the next operation; or the crude product can be get by pouring the supernatant liquid of the reaction mixture after standing a while and which is used directly in the next operation; or selecting appropriate organic solvent or a combination thereof for extraction, distillation, crystallization, column chromatography, moistening, triturating and other purification steps.

The solvent used for the reaction of the invention is not particularly restricted, any solvent is contained in the invention so long as it can dissolve the raw materials to a certain extent and doesn't inhibit the reaction. Additionally, many similar modifications in the art, equivalent replacements, or solvent, solvent composition and the solvent composition with different proportions which are equivalent to those described in the invention, all are deemed to be included in the present invention. The present invention gives the preferred solvent for each reaction step.

The amount of water in the solvent disclosed herein is not particularly restricted, i.e. the content of water in the solvent does not affect the reaction. So long as the solvent containing a certain amount of water can be used in the reaction disclosed herein, which is deemed to be included in the present invention. The amount of water in the solvent is approximately less than 0.05%, less than 0.1%, less than 0.2%, less than 0.5%, less than 5%, less than 10%, less than 25%, less than 30%, or 0%. In some embodiments, the water content of the solvent within a certain range is more favorable for the reaction; for example, in the step of using ethanol as the reaction solvent, anhydrous ethanol is more favorable for the reaction. In some embodiments, the water content of the solvent exceeding a certain range may affect the the reaction status (for example, the yield of the reaction), but does not affect the reaction initiation.

### General Synthesis and Determination Methods

In the present invention, if the chemical name of the compound doesn't match the corresponding structure, the compound is characterized by the corresponding structure.

Persons skilled in the art will recognize that the chemical reactions described may be readily adapted to prepare a number of the compounds disclosed herein, and similar compounds. The invention can be realized by modifications by those skilled in the art, e.g., by appropriately protecting groups, by utilizing other suitable reagents known in the art other than those described, and/or by making routine modifications of reaction conditions, these common preparation method of modifications also be deemed within the scope of the present invention. Alternatively, the reactions disclosed herein or known in the art will be recognized as having applicability for preparing other compounds which are similar with the compound disclosed herein.

Generally, the compound (I) can be prepared by the methods described herein. The following examples are presented to further exemplify the invention.

The structures of the compounds were identified by nuclear magnetic resonance (e.g., ¹H-NMR and ¹³C-NMR). ¹H-NMR and ¹³C-NMR chemical shifts (6) were recorded as ppm (10-6). Measure of ¹H-NMR and ¹³C-NMR are performed, respectively, on Bruker Ultrashield—400 nuclear magnetic resonance spectrometer and Bruker Avance III HD 600 nuclear magnetic resonance spectrometer using deuterated chloroform (CDCl₃), deuterated methanol (CD₃OD) or deuterated DMSO (DMSO-d₆) as a solvent and TMS (0 ppm) or deuterated chloroform (7.26 ppm) as the reference standard. When peak multiplicities are reported, the following abbreviations are used: s (singlet), d (doublet), t (triplet), m (multiplet), br (broadened), dd (doublet of doublets), dt (doublet of triplets), ddd (doublet of doublet of doublets), ddt (doublet of doublet of triplets), td (triplet of doublets), brs (broadened singlet). Coupling constants J, when given, were reported in Hertz (Hz).

MS spectra were determined on Agilen-6120 Quadrupole LC/MS mass spectrometer;

The thin-layer silica gel used was Yantai Huanghai HSGF254 silica gel plate.

The silica gel used in column chromatography generally was Qingdao Ocean Chemical Factory 200˜300 mesh or 300˜400 mesh silica gel.

The staring materials of the present invention were known or purchased from Shanghai Accela Company, Energy Company, J&K, Chengdu Aiertai Company, Alfa Company and the like, or they could be prepared by the conventional synthesis methods in the prior art.

Unless otherwise stated, the reactions disclosed herein were carried out in a nitrogen atmosphere.

The term “nitrogen atmosphere” refers to such an atmosphere that a reaction flask was equipped with a balloon or a stainless steel autoclave filled with about 1 L nitrogen.

The term “hydrogen atmosphere” refers to such an atmosphere that a reaction flask was equipped with a balloon or a stainless steel autoclave filled with about 1 L hydrogen.

Unless otherwise stated, the solution used in the examples disclosed herein was an aqueous solution.

Unless otherwise stated, the reaction temperature was room temperature.

The reaction process in the examples was monitored by thin layer chromatography (TLC). The solvent system for development of a TLC plate comprised dichloromethane and methanol, dichloromethane and ethyl acetate, petroleum ether and ethyl acetate. The volume ratio of the solvents in the solvent system was adjusted according to the polarity of the compounds.

The elution system of column chromatography comprised: A: petroleum ether (or N-hexane, cyclohexane or n-heptane, etc.) and ethyl acetate, B: dichloromethane and ethyl acetate, C: dichloromethane and methanol. The volume ratio of the solvents in the elution system was adjusted according to the polarity of the compounds, and sometimes it was also adjusted by adding a basic agent such as aqueous ammonia or an acidic agent such as acetic acid.

HPLC refers to High Performance Liquid Chromatography;

HPLC was determined on Agilent 1200DAD high pressure liquid chromatography spectrometer (Zorbax Eclipse Plus C18 150×4.6 mm chromatographic column).

The test condition of HPLC: the run time was 30 minutes (min); the column temperature was 35° C.; the detection was carried out at the wavelength of 210 nm and 254 nm using PDA detector;

the mobile phase was H₂O (A) and acetonitrile (B); and the flow rate was 1.0 L/min.

The following abbreviations are used throughout the specification:

| | |
|---|---|
| TEMPO | 2,2,6,6-tetramethylpiperidinooxy |
| KBr | potassium bromide |
| NaClO | sodium hypochlorite |
| (R)-BINOL | (R)-(+)-1,1′-bi-2-naphthol |
| Mass % | mass percent |
| LiCl | lithium chloride |
| h | hour(s) |
| i-PrMgCl · LiCl | Isopropyl magnesium chloride |
| | lithium chloride |
| Ti(O-i-Pr)₄ | tetraisopropyl titanate |
| IBX | 2-iodoxybenzoic acid |
| DMSO | dimethylsulfoxide |
| DIPEA | N,N-diisopropylethylamine |
| DCM | dichloromethane |
| min | minute(s) |
| r.t. | room temperature |

### DESCRIPTION OF THE DRAWINGS

FIG. 1 is an X-ray powder diffraction pattern of the crystal form A of the compound (VIIIb) of the present invention.

### EXAMPLES

The examples of the present invention provide the methods for preparing optically pure (1R,2S,3S,4R,5S)-5-[4-chloro-3-[(4-ethoxyphenyl)methyl]phenyl)-1-[(1R)-1-hydroxyethyl]-6,8-dioxbicyclo[3.2.1]octane-2,3,4-triol (I). Skilled in the art can learn from this invention to properly improve the process parameters to implement the preparation method. Of particular note is that all similar substitutions and modifications to the skilled person is obvious, and they are deemed to be included in the present invention. Related person can clearly realize and apply the techniques disclosed herein by making some changes, appropriate alterations or combinations to the methods without departing from spirit, principles and scope of the present disclosure.

In order to further understand the invention, it is detailed below through examples.

### EXAMPLES

### Example 1

### tert-butyl 4-[(2R,3S,4S,5R)-2,3,4-tribenzyloxy-5-(benzyloxymethyl)-6-(2,2-dimethyl propionyloxy)-5-hydroxy-hexanoyl]piperazine-1-carboxylate

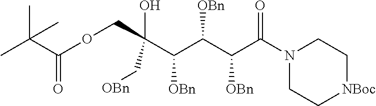 [SMILES: CC(C)(C)OC(=O)N1CCN(C(=O)[C@H](OCc2ccccc2)[C@@H](OCc2ccccc2)[C@H](OCc2ccccc2)[C@@](O)(COCc2ccccc2)COC(=O)C(C)(C)C)CC1]

### Step 1 (3R,4S,5R,6R)-3,4,5-tribenzyloxy-6-(benzyloxymethyl)tetrahydropyran-2-one

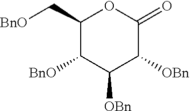 [SMILES: O=C1O[C@H](COCc2ccccc2)[C@@H](OCc2ccccc2)[C@H](OCc2ccccc2)[C@H]1OCc1ccccc1]

The compound 2,3,4,6-tetra-O-benzyl-D-glucopyranose (100.0 kg, 185.0 mol, purity: 99.0%) was dissolved in dichloromethane (466 kg), and then saturated solution of sodium bicarbonate (31.08 kg, 370 mol) in water (312 kg) was added into the solution, the mixture was cooled to 0° C. To the mixture were added potassium bromide (13.2 kg, 111 mol) and TEMPO (2.9 kg, 18.6 mol). After stirring for 1 min, NaClO solution (426 kg, 481 mol, available chlorine 4.0 mass %) was added in a whole, the resulting mixture was then further stirred for 1 h. The mixture was separated. The organic layer was washed with saturated brine (200 kg), dried over anhydrous sodium sulfate, and concentrated under reduced pressure to obtain the title compound as a yellow oil (99.6 kg, 185 mol, product content: 97.3%, yield: 100%).

### Step 2 tert-butyl 4-[(2R,3S,4R,5R)-2,3,4,6-tetrabenzyloxy-5-hydroxy-hexanoyl]piperazine-1-carboxylate

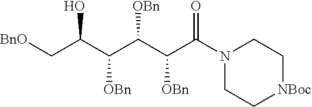 [SMILES: CC(C)(C)OC(=O)N1CCN(C(=O)[C@H](OCc2ccccc2)[C@@H](OCc2ccccc2)[C@H](OCc2ccccc2)[C@H](O)COCc2ccccc2)CC1]

At room temperature, (3R,4S,5R,6R)-3,4,5-tribenzyloxy-6-(benzyloxymethyl) tetrahydropyran-2-one (99.6 kg, 185 mol, purity: 97.3%) was dissolved in toluene (520 kg), N-Boc piperazine (86 kg, 462.5 mol) was added dropwise slowly into the solution under N₂, the mixture was kept at 30° C. and stirred for 12 h. After the reaction was complete, citric acid (53.8 kg) aqueous solution (498 kg) was added into the reaction mixture slowly. After the addition, the mixture was stirred for 20 min, stood and separated. The upper toluene solution was retained and washed with saturated brine (348.6 kg).

At room temperature, to the above toluene solution was added n-hexane (599.6 kg) dropwise, and then the mixture was stirred for 6 h. White solid was precipitated out slowly in the solution, and then the mixture was centrifuged. The filter cake was rinsed with n-hexane (99.6 kg), and the wet product was dried at 50° C. under vacuum to obtain the title compound as an off-white solid (97.9 kg, 135.1 mol, product content: 94.7%, yield: 73.0%).

### Step 3 Tert-butyl 4-[(2R,3S,4S)-2,3,4,6-tetrabenzyloxy-5-oxohexanoyl]piperazine-1-carboxylate

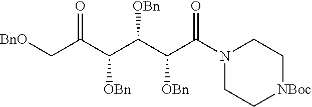 [SMILES: CC(C)(C)OC(=O)N1CCN(C(=O)[C@H](OCc2ccccc2)[C@@H](OCc2ccccc2)[C@H](OCc2ccccc2)C(=O)COCc2ccccc2)CC1]

Tert-butyl 4-[(2R,3S,4R,5R)-2,3,4,6-tetrabenzyloxy-5-hydroxy-hexanoyl]piperazine-1-carboxylate (50.0 kg, 69.0 mol, purity: 94.7%) was dissolved in toluene (129.9 kg), then DMSO (110 kg) and DIPEA (62.5 kg, 483 mol) were added into the solution. The mixture was cooled to 0° C. under N₂. Sulfur trioxide pyridine (40.0 kg, 241.5 mol) was added in batches at 0° C.˜5° C. After the addition, the mixture was stirred at 0° C.˜5° C. for 1 h. The resulting mixture was diluted with methyl tert-butyl ether (105 kg), and washed with drinking water (125 kg), and then separated. The upper organic phase was retained and washed with saturated brine (200 kg×3), and concentrated under reduced pressure to obtain the title compound as a brown oil (49.9 kg, 69.0 mol, purity: 93.8%).

### Step 4 tert-butyl 4-[(2R,3S,4S,5R)-2,3,4-tribenzyloxy-5-(benzyloxymethyl)-6-(2,2-dimethylpropionyloxy)-5-hydroxy-hexanoyl]piperazine-1-carboxylate

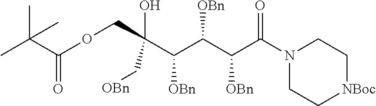 [SMILES: CC(C)(C)OC(=O)N1CCN(C(=O)[C@H](OCc2ccccc2)[C@@H](OCc2ccccc2)[C@H](OCc2ccccc2)[C@@](O)(COCc2ccccc2)COC(=O)C(C)(C)C)CC1]

Iodomethyl pivalate (23.3 kg, 96.6 mol) was dissolved in anhydrous tetrahydrofuran (58.3 kg), and the mixture was cooled to −78° C. under N₂. Tetrahydrofuran solution of i-PrMgCl-LiCl (93.7 kg, 125.6 mol, 1.3 mol/L) was added dropwise to the mixture for about 1 h. After the addition, the mixture was further stirred at −78° C. for 1 h to obtain (2,2-dimethylpropionyloxymethyl)magnesium chloride. To the reactor was added a solution of tert-butyl 4-[(2R,3S,4S)-2,3,4,6-tetrabenzyloxy-5-oxohexanoyl]piperazine-1-carboxylate (49.9 kg, 69.0 mol, product content: 93.8%) in tetrahydrofuran (149.7 kg) dropwise. After the addition, the resulting mixture was further stirred at −78° C. for 0.5 h. To the reaction solution was added a solution of glacial acetic acid (16.5 kg) in tetrahydrofuran (40 kg) dropwise to quench the reaction, then drinking water (99.8 kg) was added. The temperature of the system was controlled at 10° C. The mixture was stirred for 10 min and separated, then washed with 10% brine (300 kg). To the mixture was added methyl tert-butyl ether (25.0 kg) and stirred evenly, then n-heptane (75 kg) was added. The resulting mixture was stirred for 3 h and then centrifuged. The filter cake was rinsed with n-heptane (25 kg) and dried under vacuum at 45° C. for 6 h to obtain the title product as an off-white solid (35.3 kg, 42.1 mol, product content: 98.7%, the total yield of step 3 and step 4 is: 61%).

Other purification methods of the title compound in Step 4:

At r.t., solvent A was added into a reaction flask containing the crude product of the title compound in step 4 or its tetrahydrofuran solution, the mixture was stirred to dissolve/uniform at a certain temperature, and then solvent B was added dropwise. The resulting mixture was stirred at r.t., and an off-white solid was precipitated out. The mixture was further stirred for 3 h, and the solid precipitated out completely. The resulting mixture was filtered with suction to obtain the purified title compound as an off-white solid.

Solvent A, solvent B, the volume ratio (solvent A/solvent B), dissolution temperature, precipitation temperature, precipitation time and the test results of Tests 1-11 are listed in table 1.

TABLE 1

| | | | volume | Dissolu- | | |
|---|---|---|---|---|---|---|
| | | | ratio | tion | Test results | |
| | | | (solvent A: | Tem- | Product | |
| Test | Solvent A | Solvent B | solvent B) | perature | content | Yield |
| 1 | toluene | n-heptane | 1:4 | r.t. | 96.5% | 79.0% |
| 2 | toluene | n-heptane | 1:5 | r.t. | 95.5% | 80.5% |
| 3 | toluene | n-heptane | 1:3 | r.t. | 97.7% | 75.7% |
| 4 | toluene | n-heptane | 1:8 | r.t. | 92.6% | 84.0% |
| 5 | toluene | n-hexane | 1:5 | r.t. | 95.0% | 81.1% |
| 6 | ethyl acetate | n-heptane | 1:3 | 60° C. | 97.0% | 71.2% |
| 7 | ethyl acetate | n-heptane | 1:2 | 60° C. | 97.1% | 76.4% |
| 8 | ethyl acetate | n-hexane | 1:2 | 60° C. | 97.0% | 77.0% |
| 9 | methyl tert- | n-heptane | 1:3 | r.t. | 96.1% | 83.0% |
| | butyl ether | | | | | |
| 10 | acetone | water | 6:1 | 35° C. | 98.4% | 76.2% |
| 11 | acetone | water | 4:1 | 35° C. | 98.7% | 74.6% |

Note:
The yield in Table 1 refers to the total yield of step 3 and step 4.

### Test 12:

To the crude product (10 g, 11.93 mmol) of the title compound in step 4 was added ethyl acetate (30 g) and the temperature of the mixture was raised to 65° C. The mixture was cooled to r.t. when the product was dissolved. n-heptane (30 g) was added, and the mixture was stirred for 3 h and then centrifuged. The filter cake was rinsed with a mixed solvent of n-heptane (6 g) and ethyl acetate (3 g), the resulting product was dried under vacuum at 45° C. to obtain the product as off-white solid (7.42 g, 8.85 mol, product content: 99.1%, total yield of step 3 and step 4: 74.2%).

Specifically, the obtained product is crystal form A; the identification method of the crystal form A is as follows:

X-ray powder diffraction (XRPD) patterns were collected on a Dutch PANalytical Empyrean X-ray diffractometer equipped with an automated 3*15 zero background sample holder with a transflective sample stage. The used radiation source was (Cu, kα, Kα1 (A): 1.540598; Kα2 (Å): 1.544426; Kα2/Kα1 intensity ratio: 0.50), wherein the voltage was set at 45 KV and the current was set at 40 mA. The beam divergence of X-rays, i.e., the effective size of the X-ray confinement on the sample, was 10 mm. The θ-θ continuous scanning mode was adopted to obtain an effective 2θ range of 3°˜60°. An appropriate amount of powdered sample was placed in the circular groove of the zero background sample holder under about 18° C.˜32° C., the sample was lightly pressed with a clean glass slide to obtain a flat surface. The zero background sample frame was fixed. The sample to be tested was used to generate a traditional XRPD pattern within the range of 2θ±0.2° of 3-60° with a scanning step of 0.0168°. Use Data Collector software to collect data, HighScore Plus software to process data, Data Viewer software to read data.

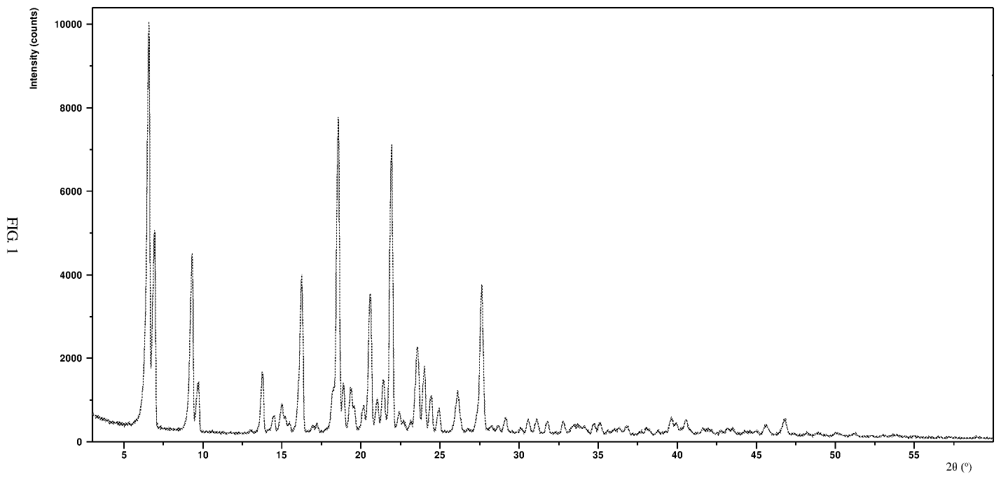

The product obtained in Test 12 is crystal form A, which has diffraction peaks at the following 2θ angles: 6.52°+0.2°, 6.890 0.2°, 9.25°±0.2°, 9.66°±0.2°, 13.72°±0.2°, 14.44°±0.2°, 14.97°±0.2°, 15.30°±0.2°, 16.18°±0.2°, 16.95°±0.2°, 17.19°±0.2°, 18.21°±0.2°, 18.44°±0.2°, 18.56°±0.2°, 18.86°±0.2°, 19.33°±0.2°, 19.56°±0.2°, 20.13°±0.2°, 20.53°±0.2°, 20.98°±0.2°, 21.39°±0.2°, 21.87°±0.2°, 22.38°±0.2°, 22.67°±0.2°, 23.12°±0.2°, 23.52°±0.2, 23.95°±0.2°, 24.38°±0.2°, 24.88°±0.2°, 26.07°±0.2°, 27.58°±0.2°, 28.65°±0.2°, 29.11°±0.2°, 30.08°±0.2°, 30.54°±0.2°, 31.07°±0.2°, 31.74°±0.2°, 32.76°±0.2°, 33.57°±0.2°, 34.11°±0.2°, 34.69°±0.2°, 35.06°±0.2°, 36.76°±0.2°, 38.05°±0.2°, 38.74°±0.2°, 39.58°±0.2°, 39.89°±0.2°, 40.51°±0.2°, 41.75°±0.2°, 43.31°±0.2°, 44.87°±0.2°, 45.59°±0.2°, 46.74°±0.2°, 48.15°±0.2°, 48.85°±0.2°, 50.06°±0.2°, 51.15°±0.2°, 52.95°±0.2°, 53.80°±0.2°. Specifically, the crystal form A has an X-ray powder diffraction pattern substantially as shown in FIG. 1.

### Example 2

### tert-butyl 4-[(2R,3S,4S,5S)-2,3,4-tribenzyloxy-5-(benzyloxymethyl)-5-hydroxy-6-oxo-hexanoyl]piperazine-1-carboxylate

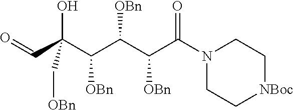 [SMILES: CC(C)(C)OC(=O)N1CCN(C(=O)[C@H](OCc2ccccc2)[C@@H](OCc2ccccc2)[C@H](OCc2ccccc2)[C@](O)(C=O)COCc2ccccc2)CC1]

### Step 1 tert-butyl 4-[(2R,3S,4S,5S)-2,3,4-tribenzyloxy-5-(benzyloxymethyl)-5,6-dihydroxy-hexanoyl]piperazine-1-carboxylate

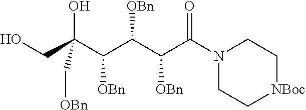 [SMILES: CC(C)(C)OC(=O)N1CCN(C(=O)[C@H](OCc2ccccc2)[C@@H](OCc2ccccc2)[C@H](OCc2ccccc2)[C@](O)(CO)COCc2ccccc2)CC1]

### Test 1:

tert-butyl 4-[(2R,3S,4S,5R)-2,3,4-tribenzyloxy-5-(benzyloxymethyl)-6-(2,2-dimethylpropionyloxy)-5-hydroxy-hexanoyl]piperazine-1-carboxylate (2.0 kg, 2.38 mol, purity: 98.7%) prepared according to the method of Example 1, toluene (10.0 L) and dimethyl sulfoxide (0.4 L) were added into a reaction kettle. The mixture was cooled to −8° C., and sodium methoxide (0.16 kg, 2.86 mol) was added in a whole. Then the mixture was stirred at −8° C. for 2 h. After the reaction was complete, saturated ammonium chloride solution (3.2 L) was added to the reaction solution. The resulting mixture was stirred for 30 min and then separated. The organic phase was washed with water (8.0 L) and saturated brine (2.4 L) in sequence, and concentrated under reduced pressure to obtain the title compound as a light yellow oil (1.80 kg, 2.38 mol, product content: 96.1%, yield: 100%).

### Tests 2-7:

tert-butyl 4-[(2R,3S,4S,5R)-2,3,4-tribenzyloxy-5-(benzyloxymethyl)-6-(2,2-dimethylpropionyloxy)-5-hydroxy-hexanoyl]piperazine-1-carboxylate (purity: 96.3%) was added to a suitable solvent (6 mL/g), and the mixture was cooled to −8° C. Alkaline reagent was added in a whole. The mixture was reacted at a certain temperature. After the reaction was complete, referring to the work-up of Test 1 of this step 1, the title compound as a pale yellow oil was obtained.

Alkaline reagent, solvent, reaction temperature, reaction time and the test results of Tests 2-7 are listed in Table 2. Wherein, the amount of alkaline reagent is a molar equivalent calculated based on the reaction substrate tert-butyl 4-[(2R,3S,4S,5R)-2,3,4-tribenzyloxy-5-(benzyloxymethyl)-6-(2,2-dimethylpropionyloxy)-5-hydroxy-hexanoyl]piperazine-1-carboxylate. For example, in Test 2, when the amount of the reaction substrate is 1 mol, the amount of the alkaline reagent sodium methoxide is 1.2 mol.

TABLE 2

| | | | | | | Test results | |
|---|---|---|---|---|---|---|---|
| | | The amount of | | | | | Raw |
| | Alkaline | Alkaline | | Reaction | Reaction | Product | material |
| Test | reagent | reagent | Solvent | Temperature | Time | content | residue |
| 2 | Sodium | 1.2 eq | Toluene/dimethyl | −8° C. | 1.5 h | 93.43% | 2.02% |
| | methoxide | | sulfoxide (V/V) = 20/1 | | | | |
| 3 | Sodium | 1.5 eq | Toluene/dimethyl | −8° C. | 1.5 h | 92.63% | 1.38% |
| | methoxide | | sulfoxide (V/V) = 20/1 | | | | |
| 4 | Sodium | 1.5 eq | Toluene/dimethyl | −15° C. | 1.5 h | 93.14% | 1.22% |
| | methoxide | | sulfoxide (V/V) = 20/1 | | | | |
| 5 | Sodium | 1.5 eq | Toluene/dimethyl | 5° C. | 1.5 h | 86.71% | 0.70% |
| | methoxide | | sulfoxide (V/V) = 20/1 | | | | |
| 6 | Sodium | 1.5 eq | Toluene/dimethyl | −8° C. | 1.5 h | 89.42% | 1.29% |
| | ethoxide | | sulfoxide (V/V) = 20/1 | | | | |
| 7 | Sodium | 1.5 eq | toluene | −8° C. | 20 h | 86.3% | 8.70% |
| | methoxide | | | | | | |

### Step 2

### Tert-butyl [(2R,3S,4S,5S)-2,3,4-tribenzyloxy-5-(benzyloxymethyl)-5-hydroxy-6-oxo-hexanoyl]piperazine-1-carboxylate

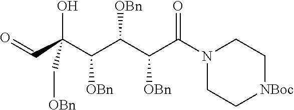 [SMILES: CC(C)(C)OC(=O)N1CCN(C(=O)[C@H](OCc2ccccc2)[C@@H](OCc2ccccc2)[C@H](OCc2ccccc2)[C@](O)(C=O)COCc2ccccc2)CC1]

tert-butyl 4-[(2R,3S,4S,5S)-2,3,4-tribenzyloxy-5-(benzyloxymethyl)-5,6-dihydroxy-hexanoyl]piperazine-1-carboxylate (1.80 kg, 2.38 mol, purity: 96.1%) prepared in test 1 of step 1 of Example 2 was dissolved in a mixed solvent of toluene (5.4 L) and dimethyl sulfoxide (3.6 L), N,N-diisopropylethylamine (2.16 kg, 16.69 mol) was added to the mixture under N₂. The mixture was cooled to −5° C. Sulfur trioxide pyridine complex (1.22 kg, 7.2 mol) was added in batches, and then the mixture was reacted at −5° C. for 1 h. After the reaction was complete, water (9.0 L) was added to the reaction mixture and the resulting mixture was warmed to r.t. The organic phase was washed with saturated brine (3.9 L×3), dried over anhydrous sodium sulfate, and then filtered and concentrated under reduced pressure to obtain the title compound as a pale yellow sticky (1.79 kg, 2.38 mol, product content: 87.8%, yield: 100%).

### Example 3

### (2R,3S,4S,5R,6R)-2,3,4-tribenzyloxy-5-(benzyloxymethyl)-5,6-dihydroxy-1-(piperazin-1-yl) heptan-1-one

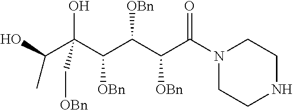 [SMILES: C[C@@H](O)[C@](O)(COCc1ccccc1)[C@@H](OCc1ccccc1)[C@H](OCc1ccccc1)[C@@H](OCc1ccccc1)C(=O)N1CCNCC1]

### Test 1:

At room temperature, dichloromethane (10.74 kg) was added to a reaction kettle, and then isopropyl titanate (2.70 kg, 9.52 mol) was added under N₂. The mixture was cooled to −10° C. To the mixture a solution of methylmagnesium bromide in 2-methyltetrahydrofuran (3.81 kg, 10.39 mol, 3 mol/L) was added dropwise, and the resulting mixture was cooled to −10° C. A solution of tert-butyl [(2R,3S,4S,5S)-2,3,4-tribenzyloxy-5-(benzyloxymethyl)-5-hydroxy-6-oxo-hexanoyl]piperazine-1-carboxylate (1.79 kg, 2.38 mol, purity: 87.8%) prepared by step 2 of Example 2 in dichloromethane (10.74 kg) was added dropwise to the mixture. The temperature in the reaction kettle was controlled at −10° C.˜0° C. during the dropping process. After the addition, the mixture was continued to stir for 20 min and then heated to 25±5° C. The mixture was further stirred for 12 h, heated up to 32° C., and then further stirred for 24 h. The resulting mixture was cooled to −15° C. and slowly added to a mixed solution of concentrated hydrochloric acid (3.58 kg) and water (8.95 kg) that had been cooled to 2° C. in advance. The resulting mixture was stirred for 1 h and then separated. The organic layer was washed with dilute hydrochloric acid (concentrated hydrochloric acid 1.79 kg, and water 8.95 kg) and saturated sodium bicarbonate solution (7.5 kg) in sequence. Saturated sodium bicarbonate solution (7.5 kg) was added to the organic layer, and the resulting mixture was stirred for 10 h and then separated. The organic layer was washed with saturated brine (3.4 kg), dried with anhydrous sodium sulfate, filtered and concentrated under reduced pressure to obtain a brown sticky (1.59 kg, 2.38 mol, product content: 86.3%, yield: 100%, ee value: 93.3%).

### Tests 2-21

At room temperature, to a mixture of a suitable solvent (6.0 L/kg) and (R)-BINOL were optionally added isopropyl titanate under N₂, the resulting mixture was cooled to −10° C., then methylating reagent was added. tert-butyl [(2R,3S,4S,5S)-2,3,4-tribenzyloxy-5-(benzyloxymethyl)-5-hydroxy-6-oxo-hexanoyl]piperazine-1-carboxylate was dissolved in a solvent and the mixture was added dropwise to the methylating reagent solution prepared above at −10° C. After the addition, the mixture was continued to stir for 20 min and then further stirred at a certain reaction temperature (such as 25±5° C.) for 12 h. The reaction temperature was raised to 32° C. and the mixture was stirred for 12 h. After the reaction was complete, the title compound was obtained as a brown sticky referring to the work-up of Test 1.

The solvent, methylating reagent, the amount of methylating agent, the amount of isopropyl titanate, the amount of (R)-BINOL and the test results of Tests 2-21 are listed in table 3. Wherein, the amount of reagent is a molar equivalent calculated based on the reaction substrate tert-butyl [(2R,3S,4S,5S)-2,3,4-tribenzyloxy-5-(benzyloxymethyl)-5-hydroxy-6-oxo-hexanoyl]piperazine-1-carboxylate.

TABLE 3

| | | the amount of | | The amount of | (R)-BINOL | Test results | | |
|---|---|---|---|---|---|---|---|---|
| | | isopropyl | methylating | methylating | The | ee | Product | Reaction |
| Test | Solvent | titanate | agent | agent | amount of | value | content | status |
| 1 | toluene | 1.4 eq | Zn(Me)₂ | 4.0 eq | 0.2 eq | 88.3% | 76.1% | complete |
| 2 | DCM | 1.4 eq | Zn(Me)₂ | 4.0 eq | 0.1 eq | 89.5% | 77.5% | complete |
| 3 | DCM | 2.0 eq | Zn(Me)₂ | 4.0 eq | 0.1 eq | 92.0% | 71.8% | complete |
| 4 | toluene | 2.0 eq | Zn(Me)₂ | 4.0 eq | 0.1 eq | 91.1% | 74.2% | complete |
| 5 | tetrahydrofuran | 1.4 eq | Zn(Me)₂ | 4.0 eq | 0.1 eq | 80.3% | 69.0% | complete |
| 6 | toluene | 1.4 eq | Zn(Me)₂ | 4.0 eq | 0.05 eq | 88.5% | 75.3% | complete |
| 7 | toluene | 1.4 eq | Zn(Me)₂ | 4.0 eq | 0.2 eq | 88.4% | 80.2% | complete |
| 8 | DCM | 1.4 eq | Zn(Me)₂ | 4.0 eq | 0.1 eq | 89.3% | 83.3% | complete |
| 9 | toluene | 1.4 eq | Zn(Me)₂ | 5.0 eq | 0.1 eq | 89.5% | 83.2% | complete |
| 10 | toluene | 1.4 eq | Zn(Me)₂ | 6.0 eq | 0.05 eq | 83.5% | 78.9% | complete |
| 11 | methyl tert-butyl ether | 1.4 eq | Zn(Me)₂ | 4.0 eq | 0.1 eq | 80.2% | 80.5% | complete |
| 12 | toluene | 2.0 eq | MeMgBr | 5.0 eq | — | 74.2% | 65.4% | complete |
| 13 | DCM | 1.0 eq | MeMgBr | 5.0 eq | — | 69.3% | 64.5% | complete |
| 14 | DCM | 2.0 eq | MeMgBr | 5.0 eq | — | 87.3% | 74.2% | complete |
| 15 | DCM | 3.0 eq | MeMgBr | 5.0 eq | — | 90.1% | 84.8% | complete |
| 16 | DCM | 4.0 eq | MeMgBr | 5.0 eq | — | 93.4% | 86.3% | complete |
| 17 | DCM | — | MeMgBr | 5.0 eq | — | 2.0% | 46.6% | complete |
| 18 | tetrahydrofuran | 4.0 eq | MeMgBr | 5.0 eq | — | 51.2% | 58.2% | complete |
| 19 | 2-methyltetrahydrofuran | 4.0 eq | MeMgBr | 5.0 eq | — | 60.0% | 62.1% | complete |
| 20 | toluene | 4.0 eq | MeMgBr | 5.0 eq | | 73.1% | 67.1% | complete |
| 21 | methyl tert-butyl ether | 4.0 eq | MeMgBr | 5.0 eq | — | 70.5% | 64.2% | complete |

Note:
In Table 3, “—” means no addition or absence; MeMgBr refers to 3 mol/L methylmagnesium bromide tetrahydrofuran solution; Zn(Me)₂ refers to 1.0 mol/L dimethylzinc n-hexane solution; “complete” refers to the reaction substrate tert-butyl [(2R,3S,4S,5S)-2,3,4-tribenzyloxy-5-(benzyloxymethyl)-5-hydroxy-6-oxo-hexanoyl]piperazine-1-carboxylate was completely consumed.

### Example 4

### (2R,3S,4S,5R,6R)-2,3,4-tribenzyloxy-5-(benzyloxymethyl)-5,6-dihydroxy-1-(piperazine-1-yl)heptan-1-one methanesulfonate

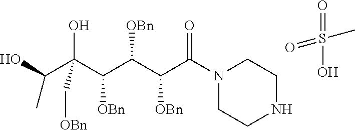

Ethyl acetate (5.74 kg) and methyl tert-butyl ether (2.35 kg) were added to a reaction kettle containing (2R,3S,4S,5R,6R)-2,3,4-tribenzyloxy-5-(benzyloxymethyl)-5,6-dihydroxy-1-(piperazin-1-yl)heptan-1-one (1.59 kg, 2.38 mol, product content: 86.3%). The mixture was stirred until dissolution. Methanesulfonic acid was added to the mixture to adjust pH to 5.5˜7.0. After the addition, the mixture was stirred for 12 h and centrifuged. The wet product was rinsed with ethyl acetate/methyl tert-butyl ether (V/V=2/1, 2.77 kg) and heptane (3.18 kg). The wet product was returned to the reaction kettle, and n-heptane (3.50 kg) was added. The resulting mixture was stirred for 12 h, centrifuged to dryness, and dried under vacuum at 35±5° C. to obtain an off-white solid (1.17 kg, 1.51 mol, product content: 95.55) %, yield: 64.0%).

### Example 5

### (2R,3S,4S)-2,3,4-tribenzyloxy-4-[(4R,5R)-4-(benzyloxymethyl)-2,2,5-trimethyl-1,3-dioxolane-4-yl)-1-(piperazin-1-yl)butane-1-one oxalate

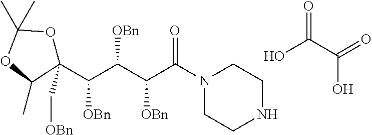

The (2R,3S,4S,5R,6R)-2,3,4-tribenzyloxy-5-(benzyloxymethyl)-5,6-dihydroxy-1-(piperazin-1-yl)-heptan-1-one methanesulfonate (1.17 kg, 1.51 mol, product content: 95.55%) prepared in Example 4 was dissolved in toluene (4.68 kg), then 2,2-dimethoxypropane (4.18 kg) was added, and the mixture was cooled to 8° C. Methanesulfonic acid (0.03 kg) was added, and the mixture was stirred at 8° C. for 2 h. After the reaction, the mixture was washed with saturated sodium bicarbonate solution (4.0 kg) and water (4.68 kg) in sequence. The organic phase was directly concentrated under reduced pressure without drying to obtain a brown syrup. Methyl tert-butyl ether (3.51 kg) was added to the syrup, the mixture was heated to 50° C. and stirred to dissolution, then cooled to 20° C. Anhydrous oxalic acid (0.14 kg) was added to the mixture. After the addition, the mixture was further stirred for 12 h and centrifuged. The filter cake was rinsed with methyl tert-butyl ether (1.17 L) and dried at 35±5° C. for 12 h to obtain an off-white solid (0.92 kg, purity: 95.58%, yield: 76.3%).

The methanesulfonic acid of Example 4 can be replaced with other acids, such as sulfuric acid, citric acid, oxalic acid, etc. The compound (2R,3S,4S)-2,3,4-tris(benzyloxy)-4-[(4R,5R)-4-(benzyloxymethyl)-2,2,5-trimethyl-1,3-dioxolan-4-yl)-1-(piperazin-1-yl)butane-1-one oxalate can also be prepared according to the methods of Examples 4 and 5.

### Example 6

### (2R,3S,4S)-1-(4-allylpiperazin-1-yl)-2,3,4-tribenzyloxy-4-[(4R,5R)-4-(benzyloxymethyl)-2,2,5-trimethyl-1,3-dioxolane-4-yl)butan-1-one

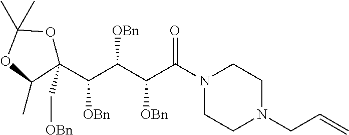 [SMILES: C=CCN1CCN(C(=O)[C@H](OCc2ccccc2)[C@@H](OCc2ccccc2)[C@H](OCc2ccccc2)[C@]2(COCc3ccccc3)OC(C)(C)O[C@@H]2C)CC1]

(2R,3S,4S)-2,3,4-tribenzyloxy-4-[(4R,5R)-4-(benzyloxymethyl)-2,2,5-trimethyl-1,3-dioxolane-4-yl)-1-(piperazin-1-yl)butane-1-one oxalate (0.92 kg, purity: 95.58%) prepared according to the method of Example 5 was dissolved in methyl tert-butyl ether (3.68 kg), to the mixture were added water (2.48 kg) and 3-bromopropene (0.15 kg) in sequence. The mixture was stirred at 25° C. for 18 h, then stood and separated. The upper organic phase was retained and washed with water (2.76 kg) and saturated brine (1.06 kg) in sequence, dried with anhydrous sodium sulfate, filtered and concentrated under reduced pressure to obtain a brown syrup (0.86 kg, 1.15 mol, product content: 94.04%, yield: 100%).

### Example 7

### (2R,3S,4S)-1-(4-benzylpiperazin-1-yl)-2,3,4-tribenzyloxy-4-[(4R,5R)-4-(benzyloxymethyl)-2,2,5-trimethyl-1,3-dioxolane-4-yl)butan-1-one

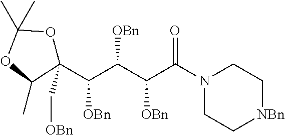 [SMILES: C[C@H]1OC(C)(C)O[C@@]1(COCc1ccccc1)[C@@H](OCc1ccccc1)[C@H](OCc1ccccc1)[C@@H](OCc1ccccc1)C(=O)N1CCN(Cc2ccccc2)CC1]

(2R,3S,4S)-2,3,4-tribenzyloxy-4-[(4R,5R)-4-(benzyloxymethyl)-2,2,5-trimethyl-1,3-dioxolane-4-yl)-1-(piperazin-1-yl)butane-1-one oxalate (4.1 g, 5.14 mmol, purity: 95.58%) prepared according to the method of Example 5 was dissolved in methyl tert-butyl ether (16.4 g), to the mixture were added water (11.07 g) and benzyl bromide (0.97 g, 5.65 mmol) in sequence. The mixture was stirred at 25° C. for 12 h, then stood and separated. The upper organic phase was retained and washed with water (12.3 g) and saturated brine (4.7 g) in sequence, dried with anhydrous sodium sulfate, filtered and concentrated under reduced pressure to obtain a brown syrup (4.11 g, 5.14 mmol, product content: 92.31%, yield: 100%).

### Example 8

### (2R,3S,4S)-2,3,4-tribenzyloxy-4-[(4R,5R)-4-(benzyloxymethyl)-2,2,5-trimethyl-1,3-dioxopent-4-yl]-1-[4-(p-toluenesulfonyl)piperazin-1-yl]butan-1-one

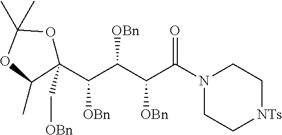 [SMILES: Cc1ccc(S(=O)(=O)N2CCN(C(=O)[C@H](OCc3ccccc3)[C@@H](OCc3ccccc3)[C@H](OCc3ccccc3)[C@]3(COCc4ccccc4)OC(C)(C)O[C@@H]3C)CC2)cc1]

### Step 1 (2R,3S,4S,5R,6R)-2,3,4-tribenzyloxy-5-(benzyloxymethyl)-5,6-dihydroxy-1-[4-(p-toluene sulfonyl)piperazin-1-yl)heptan-1-one

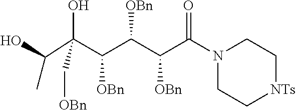 [SMILES: Cc1ccc(S(=O)(=O)N2CCN(C(=O)[C@H](OCc3ccccc3)[C@@H](OCc3ccccc3)[C@H](OCc3ccccc3)[C@@](O)(COCc3ccccc3)[C@@H](C)O)CC2)cc1]

(2R,3S,4S,5R,6R)-2,3,4-tribenzyloxy-5-(benzyloxymethyl)-5,6-dihydroxy-1-(piperazin-1-yl)heptan-1-one (17.4 g, 26.0 mmol) prepared according to the method of Example 3 and triethylamine (7.2 mL, 52 mmol) were dissolved in dichloromethane (100 mL), the mixture was cooled to 0° C., and 4-methylbenzenesulfonyl chloride was added to the mixture. The resulting mixture was moved to r.t. and stirred for 10 min, then washed with water (100 mL) and saturated sodium chloride solution (100 mL) in sequence, and concentrated under reduced pressure. The residue was purified by silica gel column chromatography (ethyl acetate/petroleum ether (v/v)=1/2-1/1) to obtain a white foamy solid (7.6 g, 9.2 mmol, yield: 35%, ee value: 93.4%).

### Step 2 (2R,3S,4S)-2,3,4-tribenzyloxy-4-[(4R,5R)-4-(benzyloxymethyl)-2,2,5-trimethyl-13-dioxolan-4-yl]-1-[4-(p-toluenesulfonyl)piperazin-1-yl]butan-1-one

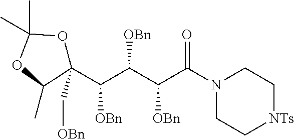 [SMILES: Cc1ccc(S(=O)(=O)N2CCN(C(=O)[C@H](OCc3ccccc3)[C@@H](OCc3ccccc3)[C@H](OCc3ccccc3)[C@]3(COCc4ccccc4)OC(C)(C)O[C@@H]3C)CC2)cc1]

(2R,3S,4S,5R,6R)-2,3,4-tribenzyloxy-5-(benzyloxymethyl)-5,6-dihydroxy-1-(4-(p-toluenesulfonyl))piperazin-1-yl)heptan-1-one (0.85 g, 1.0 mmol) was dissolved in toluene (5 mL), to the solution were added 2,2-dimethoxypropane (0.50 mL, 4.1 mmol) and methanesulfonic acid (0.04 mL, 0.6 mmol). The mixture was moved to r.t. and stirred for 20 min. The resulting mixture was washed with saturated sodium bicarbonate solution (5 mL) and saturated brine (5 mL) in sequence, and concentrated under reduced pressure to obtain a white foamy solid (0.89 g, 1.0 mmol, yield: 100%).

### Example 9

### (2R,3S,4S,5R)-3,4,5-tribenzyloxy-2-(benzyloxymethyl)-2-hydroxy-6-oxo-6-(4-triphenylmethyl-piperazin-1-yl)hexyl pivalate

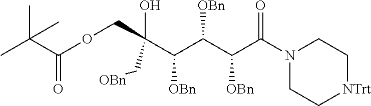

### Step 1 (2R,3S,4R,5R)-2,3,4,6-tetra(benzyloxy)-5-hydroxy-1-(piperazin-1-yl)hexan-1-one

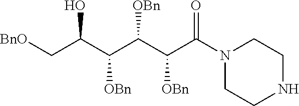 [SMILES: O=C([C@H](OCc1ccccc1)[C@@H](OCc1ccccc1)[C@H](OCc1ccccc1)[C@H](O)COCc1ccccc1)N1CCNCC1]

The compound (3R,4S,5R,6R)-3,4,5-tribenzyloxy-6-(benzyloxymethyl) tetrahydrofuran-2-one (50.0 g, 92.8 mmol) was dissolved in DMSO (200 mL), to the solution was added piperazine (24 g, 278.6 mmol), and the mixture was stirred at r.t. for 10 h. Toluene (400 mL) was added to dilute the mixture. The resulting mixture was washed with water (400 mL×3) and saturated sodium chloride solution (400 mL) in sequence, concentrated under reduced pressure to about 100 mL of the remaining mixture. Then n-heptane (200 mL) was added dropwise with stirring. After the addition, the mixture was stirred overnight at r.t. The upper layer was separated and removed, and the lower mixture was dried under vacuum to obtain a pale yellow syrup (34.0 g, 54.4 mmol, yield: 58.6%).

### Step 2 (2R,3S,4R,5R)-2,3,4,6-tetra(benzyloxy)-5-hydroxy-1-(4-tritylpiperazine-1-yl)hexan-1-one

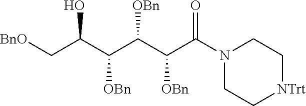

Triethylamine (281 mL, 1820 mmol) was added to a solution of (2R,3S,4R,5R)-2,3,4,6-tetra(benzyloxy)-5-hydroxy-1-(piperazin-1-yl)hexan-1-one (379.5 g, 607.4 mmol) in dichloromethane (1900 mL), [chloro(diphenyl)methyl]benzene (203.2 g, 728.8 mmol) was added with stirring, and the mixture was stirred at r.t. Water (1.5 L) was added to the mixture, and then the mixture was stirred for 10 min, stood and separated. The lower organic phase was washed once with 15% sodium chloride aqueous solution, dried with anhydrous sodium sulfate, filtered, and concentrated under reduced pressure. The residue was purified by silica gel flash column chromatography (EA/PE, volume ratio 10%-33%) to obtain a yellow oil (451 g, purity: 90.0%, 520.1 mmol, yield: 85.63%).

### Step 3 (2R,3S,4S)-2,3,4,6-tetrakis(benzyloxy)-1-(4-tritylpiperazin-1-yl)hexan-1,5-dione

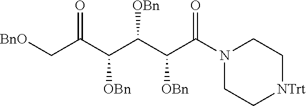

(2R,3S,4R,5R)-2,3,4,6-tetra(benzyloxy)-5-hydroxy-1-(4-tritylpiperazin-1-yl)hexan-1-one (451.0 g, 520.1 mmol) was dissolved in n-heptane (1353 mL) and DMSO (32 mL, 450 mmol), To the mixture was added N,N-diisopropylethylamine (604 mL, 3640 mmol). The mixture was cooled to 5° C. under N₂, a solution of sulfur trioxide pyridine (295.7 g, 1821 mmol) and DMSO (1353 mL, 19000 mmol) was added dropwise. After the addition, the mixture was kept the temperature and stirred for 2 h, then stood and separated. The lower layer was washed with n-heptane (1000 mL×2) and then slowly poured into ice water (200 g). To the mixture was added methyl tert-butyl ether (2.0 L), and the resulting mixture was stirred for 10 minutes. After standing, the organic phase was washed with saturated aqueous sodium chloride solution (2000 mL×2), dried over anhydrous sodium sulfate and filtered. The filtrate was concentrated under reduced pressure to obtain the title compound as an off-white solid (394 g, 455.4 mmol, yield: 87.56%).

### Step 4 (2,2-dimethylpropionyloxymethyl)magnesium chloride

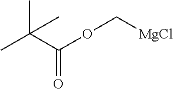

Iodomethyl pivalate (70.0 g, 289 mmol) was dissolved in anhydrous tetrahydrofuran (210 mL) and the solution was cooled to −40° C. under N₂. A solution of isopropylmagnesium chloride lithium chloride in tetrahydrofuran (230 mL, 300 mmol, 1.3 mol/L) was added dropwise to the solution for about 30 min, and the temperature was controlled at −40° C.˜−30° C. After the addition, the mixture was stirred while gradually cooled to −70° C. to obtain a gray-black liquid (50.6 g, 289 mmol, yield: 100%). The obtained Grignard reagent was directly used in the subsequent reaction (yield was calculated as 100%).

### Step 5 (2R,3S,4S,5R)-3,4,5-tribenzyloxy-2-(benzyloxymethyl)-2-hydroxy-6-oxo-6-(4-trityl piperazine-1-yl)hexyl pivalate

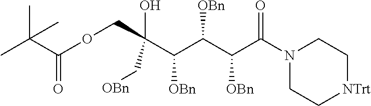

(2R,3S,4S)-2,3,4,6-tetra(benzyloxy)-1-(4-tritylpiperazin-1-yl)hexan-1,5-dione (70.0 g, 80.9 mmol) was dissolved in anhydrous tetrahydrofuran (120 mL), and then the mixture was added dropwise to the anhydrous tetrahydrofuran solution containing (2,2-dimethylpropionyloxymethyl) magnesium chloride (49.4 g, 282 mmol) prepared in the previous step at −70° C. The temperature of the system was controlled at −70° C.˜−60° C. After the addition, the resulting mixture was stirred at this temperature for 1 h. A solution of glacial acetic acid (20 mL) in tetrahydrofuran (60 mL) was added to the mixture to quench the reaction. Petroleum ether (100 mL) was added to dilute the mixture. The resulting mixture was washed with saturated sodium bicarbonate solution (300 mL) and saturated brine (300 mL) in sequence, concentrated under reduced pressure to obtain the title compound as a yellow oil (80.0 g, 81.5 mmol, yield: 100%).

### Example 10

### (2R,3S,4S,5R)-2,3,4-tribenzyloxy-5-(benzyloxymethyl)-5,6-dihydroxy-1-(4-tritylpiperazine-1-yl) hexan-1-one

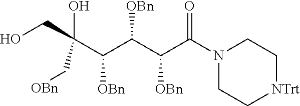

(2R,3S,4S,5R)-3,4,5-tribenzyloxy-2-(benzyloxymethyl)-2-hydroxy-6-oxo-6-(4-tritylpiperazine-1-yl)hexyl pivalate (80 g, 82 mmol) was dissolved in toluene (300 mL) and the solution was cooled to 0° C. under N₂, then solid sodium methoxide (6.6 g, 120 mmol) was added to the solution. The resulting mixture was stirred at 0° C. for 12 h. The mixture was washed with saturated sodium bicarbonate solution (200 mL) and saturated sodium chloride solution (200 mL) in sequence, and concentrated under reduced pressure to obtain a yellow foamy product (73 g, 82 mmol).

### Example 11

### (2S,3S,4S,5R)-3,4,5-tribenzyloxy-2-(benzyloxymethyl)-2-hydroxy-6-oxo-6-(4-tritylpiperazine-1-yl)hexanal

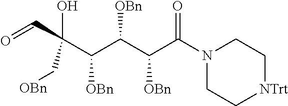

(2R,3S,4S,5R)-2,3,4-tribenzyloxy-5-(benzyloxymethyl)-5,6-dihydroxy-1-(4-tritylpiperazine-1-yl)hexan-1-one (17.0 g, 18.9 mmol) was dissolved in toluene (80 mL), to the solution were added DMSO (24 mL) and DIPEA (27 mL, 150 mmol), and the mixture was cooled to −10° C. under N₂, then to the mixture was added sulfur trioxide pyridine complex (10.6 g, 66.6 mmol). The resulting mixture was stirred at 0° C.˜5° C. for 1 h, then washed with water (100 mL) and saturated brine (100 mL×2) in sequence, and concentrated under reduced pressure to obtain a dark yellow syrup (17.0 g, 19.0 mmol, yield: 100%).

### Example 12

### (2R,3S,4S,5R,6R)-2,3,4-tribenzyloxy-5-(benzyloxymethyl)-5,6-dihydroxy-1-(4-tritylpiperazin-1-yl)heptan-1-one

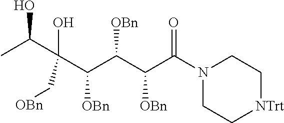

Salen (5.0 g, 9.1 mmol) was dissolved in dichloromethane (500 mL), and tetraisopropyl titanate (150 mL, 480 mmol, 95 mass %) was added to the solution under N₂. The mixture was cooled to −20° C. Then methyl magnesium bromide tetrahydrofuran solution (610 mL, 610 mmol, 1.0 mol/L) was added dropwise to the mixture for about 30 min and the temperature was controlled at −5° C.˜0° C. After the addition, the mixture was stirred for 5 min, and then a solution of (2S,3S,4S,5R)-3,4,5-tribenzyloxy)-2-(benzyloxymethyl-2-hydroxy-6-oxo-6-(4-tritylpiperazin-1-yl)hexanal (110 g, 123 mmol) in dichloromethane (300 mL) was add dropwise for about 30 minutes while the temperature was controlled at −5° C.˜0° C. The resulting mixture was moved to r.t. and stirred for 14 h, then poured into ice water containing ammonium chloride (about 400 g). To the mixture was added methyl tert-butyl ether (1.0 L), and the mixture was stirred for 5 min, and then filtered with suction. The organic phase was separated from the filtrate and washed with water (1.0 L) and saturated sodium chloride solution (1.0 L) in sequence and concentrated under reduced pressure to obtain a yellow foamy solid (112 g, 122.9 mmol, yield: 100%, ee value: 93.4%).

### Example 13

### (2R,3S,4S)-2,3,4-tribenzyloxy-4-[(4R,5R)-4-(benzyloxymethyl)-2,2,5-trimethyl-1,3-cyclopent-4-yl)-1-(4-triphenylmethylpiperazin-1-yl)butan-1-one

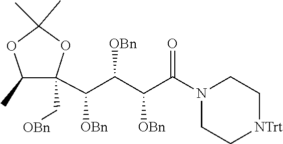

(2R,3S,4S,5R,6R)-2,3,4-tribenzyloxy-5-(benzyloxymethyl)-5,6-dihydroxy-1-(4-triphenylmethylpiperazin-1-yl)heptan-1-one (4.5 g, 4.9 mmol) was dissolved in toluene (25 mL) and the mixture was cooled to 0° C. To the mixture were added 2,2-dimethoxypropane (2.4 mL, 20 mmol) and methanesulfonic acid (0.16 mL, 2.5 mmol), and the mixture was moved to r.t. and stirred for 40 min, then washed with saturated sodium bicarbonate solution (30 mL) and saturated brine (30 mL) in sequence, and concentrated under reduced pressure to obtain a pale yellow syrup (4.7 g, 4.9 mmol, yield: 100%). The crude product was used directly in the next step.

### Example 14: [4-chloro-3-[(4-ethoxyphenyl)methyl]phenyl]magnesium bromide

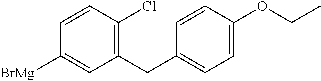

Magnesium chips (0.041 kg, 1.71 mol) and iodine pellets (0.23 g) were added into a 5 L four-neck bottle. To the mixture was added 100 mL of a solution of 5-bromo-2-chloro-4′-ethoxydiphenylmethane (0.534 kg, 1.64 mol) in anhydrous tetrahydrofuran (1.81 L), and the mixture was heated until the reaction started. Then the remaining solution of 5-bromo-2-chloro-4′-ethoxydiphenylmethane in anhydrous tetrahydrofuran was added dropwise to the mixture for about 40 min. After the addition, the mixture was further stirred for 50 min, and then cooled to r.t. to obtain a gray-black solution (1.39 mol, product purity: 91.60%, yield is calculated as 85%). The obtained Grignard reagent was directly used in the next reaction.

### Example 15

### (2R,3S,4S)-2,3,4-tribenzyloxy-4-[(4R,5R)-4-(benzyloxymethyl)-2,2,5-trimethyl-1,3-dioxolane-4-yl]-1-[4-chloro-3-[(4-ethoxyphenyl)methyl]phenyl]butan-1-one

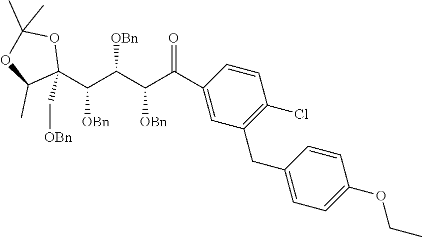 [SMILES: CCOc1ccc(Cc2cc(C(=O)[C@H](OCc3ccccc3)[C@@H](OCc3ccccc3)[C@H](OCc3ccccc3)[C@]3(COCc4ccccc4)OC(C)(C)O[C@@H]3C)ccc2Cl)cc1]

Method One:

(2R,3S,4S)-1-(4-allylpiperazin-1-yl)-2,3,4-tribenzyloxy-4-[(4R,5R)-4-(benzyloxymethyl)-2,2,5-trimethyl-1,3-dioxolane-4-yl)butan-1-one (0.74 kg, 0.99 mol, product purity: 91.98%) prepared according to the method of Example 6 was dissolved in anhydrous tetrahydrofuran (2.00 kg), and the mixture was cooled to −20° C. under N₂. A solution of [4-chloro-3-[(4-ethoxyphenyl) methyl]phenyl]magnesium bromide (1.39 mol, purity: 91.60%) in tetrahydrofuran (1.81 L) was added dropwise to the mixture for about 20 min. After the addition, the mixture was stirred at −20° C. for 30 min, and then moved to r.t. and further stirred for 2 h. After the reaction, to the mixture was added dilute hydrochloric acid solution (made from concentrated hydrochloric acid (0.41 kg) and water (1.48 kg)) to quench the reaction. The resulting mixture was extracted with n-heptane (2.00 kg), and the organic phase was washed with saturated brine (2.20 L) and concentrated under reduced pressure. Then n-heptane (13.33 L) was added to the concentrate, and the resulting mixture was stirred for 20 min and then washed with a mixed solution of methanol and water (m/m=6/1, 1.38 kg). To the n-heptane phase was added column chromatography silica gel (0.14 kg), and the mixture was stirred for 8 h and filtered with suction. The filtrate was concentrated under reduced pressure to obtain a brown oil (0.687 kg, product content: 92.4%, yield: 80.0%).

¹H NMR (400 MHz, CDCl₃) δ (ppm) 8.07 (s, 1H), 7.95 (d, 1H), 7.42 (t, 6H), 7.36-7.28 (m, 10H), 7.08 (dt, 5H), 6.93 (d, 2H), 6.82 (d, 2H), 5.27 (d, 1H), 4.91 (dd, 2H), 4.70-4.50 (m, 6H), 4.44 (dd, 2H), 4.20 (d, 1H), 4.13-3.93 (m, 6H), 3.84 (dd, 2H), 1.49 (s, 3H), 1.42 (dd, 6H), 1.25 (s, 3H).

Method Two:

(2R,3S,4S)-1-(4-benzylpiperazin-1-yl)-2,3,4-tribenzyloxy-4-[(4R,5R)-4-(benzyloxymethyl)-2,2,5-trimethyl-1,3-dioxolane-4-yl]butan-1-one (4.11 g, 5.14 mmol, purity: 92.31%) prepared according to the method of Example 7 was dissolved in anhydrous tetrahydrofuran (13.1 mL) and the mixture was cooled to −20° C. under N₂. To the mixture was added dropwise a solution of [4-chloro-3-[(4-ethoxyphenyl)methyl]phenyl]magnesium bromide in tetrahydrofuran (13.7 mL, 6.68 mmol, 0.50 mol/L, the preparation method refers to Example 14) for about 5 min. After the addition, the mixture was stirred at −20° C. for 20 min and then moved to r.t. and further stirred for 2 h. The mixture was cooled to 0° C., and then diluted hydrochloric acid solution (21 mL, 1 mmol/mL) was added. The resulting mixture was extracted with n-heptane (30 mL), and the organic phase was washed with saturated brine (15 mL) and concentrated under reduced pressure. The obtained residue was dissolved in n-heptane (60 mL), washed with methanol/water (v/v=8/1, 10 mL), and concentrated under reduced pressure to obtain a colorless oil (4.02 g, 4.63 mmol, product content: 73.0%, yield: 90%).

Method Three:

(2R,3S,4S)-2,3,4-tribenzyloxy-4-[(4R,5R)-4-(benzyloxymethyl)-2,2,5-trimethyl-1,3-dioxolpan-4-yl]-1-[4-(p-toluenesulfonyl)piperazin-1-yl]butan-1-one (0.89 g, 1.0 mmol) prepared according to the method of Example 8 was dissolved in anhydrous tetrahydrofuran (4 mL) and the mixture was cooled to −20° C. under N₂. To the mixture was added dropwise [4-chloro-3-[(4-ethoxyphenyl)methyl]phenyl]magnesium bromide (2.6 mL, 1.3 mmol, 0.50 mol/L, the preparation method refers to Example 14) for about 1 min. After the addition, the mixture was moved to r.t. and stirred for 30 min. To the mixture was added saturated ammonium chloride solution (5 mL) to quench the reaction. The resulting mixture was extracted with petroleum ether (10 mL). The organic phase was washed with saturated sodium chloride solution (10 mL). To the mixture was added concentrated hydrochloric acid (1.0 mL), and then the mixture was stirred for 3 h and separated. The organic phase was washed with saturated sodium bicarbonate solution (5 mL) and concentrated under reduced pressure. The residue was filtered through silica gel to obtain a colorless oil (0.50 g, 0.58 mmol, yield: 56%).

Method Four:

(2R,3S,4S)-2,3,4-tribenzyloxy-4-[(4R,5R)-4-(benzyloxymethyl)-2,2,5-trimethyl-1,3-cyclopent-4-yl)-1-(4-triphenylmethylpiperazin-1-yl)butan-1-one (4.7 g, 4.9 mmol) prepared according to the method of Example 13 was dissolved in anhydrous tetrahydrofuran (20 mL) and the mixture was cooled to −20° C. under N₂. To the mixture was added dropwise bromo-[4-chloro-3-[(4-ethoxyphenyl) methyl]phenyl]magnesium (14 mL, 6.4 mmol, 0.46 mol/L) prepared according to the method of Example 14 for about 2 min. After the addition, the mixture was stirred at −20° C. for 10 min and then moved to r.t. and further stirred for 20 min. To the mixture was added saturated ammonium chloride solution (30 mL) to quench the reaction, and then the resulting mixture was extracted with petroleum ether (40 mL), and the organic phase was washed with saturated brine (50 mL) and concentrated under reduced pressure. The residue was filtered through silica gel to obtain a colorless oil (3.0 g, 3.5 mmol, yield: 70%).

### Example 16

### (1R,2S,3S,4R,5S)-5-(3-(4-ethoxybenzyl)-4-chlorophenyl)-1-((1R)-1-hydroxyethyl)-6,8-dioxa-bicyclo[3.2.1]octane-2,3,4-triol

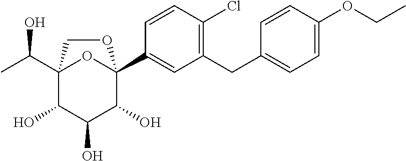 [SMILES: CCOc1ccc(Cc2cc([C@]34OC[C@]([C@@H](C)O)(O3)[C@@H](O)[C@H](O)[C@H]4O)ccc2Cl)cc1]

(2R,3S,4S)-2,3,4-tribenzyloxy-4-[(4R,5R)-4-(benzyloxymethyl)-2,2,5-trimethyl-1,3-dioxolane-4-yl]-1-[4-chloro-3-[(4-ethoxyphenyl)methyl]phenyl]butane-1-one (0.687 kg, 0.79 mol, purity: 92.4%) prepared according to method one of Example 15 was dissolved in tetrahydrofuran (0.53 kg). To the mixture were added methanol (1.63 kg), concentrated hydrochloric acid (0.31 kg, 36 mass %) and palladium/carbon (0.14 kg, 10 mass %), and the resulting mixture was stirred for 12 h under H₂. After the reaction, the mixture was filtered with suction and the filtrate was concentrated under reduced pressure. The residue was dissolved in ethyl acetate (4.12 kg), washed with water (2.75 kg), saturated sodium bicarbonate solution (1.47 kg) and saturated brine (1.37 kg) in sequence, and then concentrated under reduced pressure. The residue was recrystallized from ethanol (0.29 kg) and n-heptane (0.26 kg) to obtain a white solid (0.263 kg, 0.52 mol, product purity: 99.27%, ee value: 99%, yield: 67.1%).

MS (ESI, pos. ion) m/z: 451.2 [M+H]⁺;

¹H NMR (600 MHz, DMSO-d₆) δ (ppm): 7.41 (dd, 2H), 7.35-7.29 (m, 1H), 7.11 (d, 2H), 6.84 (d, 2H), 5.30 (d, 1H), 5.01 (d, 1H), 4.92 (d, 1H), 4.64 (d, 1H), 4.03-3.95 (m, 5H), 3.85 (p, 1H), 3.78 (d, 1H), 3.59-3.53 (m, 1H), 3.44 (dd, 1H), 3.38 (m, 1H), 1.30 (t, 3H), 1.18 (d, 3H).

The compound of Example 16 can also be prepared by other similar methods. For example, The substituents (allyl, benzyl, trityl or p-toluenesulfonyl) of piperazine in the intermediate compounds of Examples 6-8 or 13 can be replaced with other substituents, such as phenyl, diphenylmethyl, bis(4-methoxyphenyl)(phenyl)methyl, benzenesulfonyl, 4-bromobenzenesulfonyl, pyridyl, benzylsulfonyl, imidazolyl or N,N-dimethylaminosulfonyl; and then the target product can be obtained according to the method of Examples 15-16 of the present invention. The replaced intermediate compound can be prepared according to the preparation method of the intermediate compound of the present invention with appropriate adjustments, or can be prepared by a suitable synthetic method selected according to the structural characteristics of the intermediate compound.

Reference throughout this specification to “an embodiment,” “some embodiments,” “one embodiment”, “another example,” “an example,” “a specific example,” or “some examples,” means that a particular feature, structure, material, or characteristic described in connection with the embodiment or example is included in at least one embodiment or example of the present disclosure. Thus, the appearances of the above terms throughout this specification are not necessarily referring to the same embodiment or example of the present disclosure. Furthermore, the particular features, structures, materials, or characteristics may be combined in any suitable manner in one or more embodiments or examples. In addition, those skilled in the art can integrate and combine different embodiments, examples or the features of them as long as they are not contradictory to one another.

Although explanatory embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure.

1-72. (canceled)

73. A method for preparing compound (Va), comprising the step of:
reacting compound (VIa) with methylating reagent 1 through an addition reaction in the presence of isopropyl titanate to obtain compound (Va), 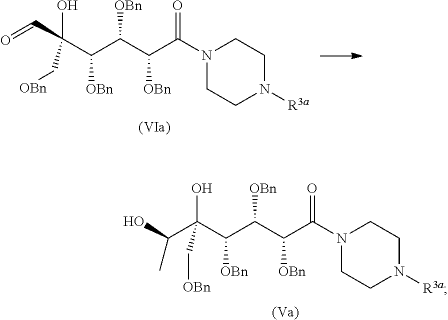
wherein, the methylating reagent 1 is methylmagnesium bromide, methylmagnesium chloride, methyllithium, trimethylaluminum or dimethyl zinc;
R^{3a} is C₂₋₈ alkyl, C₄₋₈ alkenyl, allyl, phenyl, benzyl, p-toluenesulfonyl, benzenesulfonyl, 4-bromobenzenesulfonyl, 4-nitrophenyl, 1,3-dichlorophenyl, tert-butoxycarbonyl, triphenylmethyl, bis(4-methoxyphenyl)(phenyl)methyl, diphenylmethyl, N,N-diphenylaminoacyl, pyridyl, benzylsulfonyl, imidazolyl, N,N-dimethylaminosulfonyl, N,N-dimethylaminoacyl or

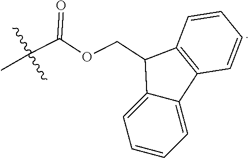 [SMILES: *C(=O)OCC1c2ccccc2-c2ccccc21]

74. The method according to claim 73, wherein the reaction is optionally carried out in the presence of a chiral ligand 1, and the chiral ligand 1 is a dihydroxy chiral ligand or a metal ligand.

75. The method according to claim 74, wherein the dihydroxy chiral ligand is R-1,1′-Bi-2-naphthol, (4R,5R)-2,2-dimethyl-a,a,a′,a′-tetraphenyl-1,3-dioxolane-4,5-dimethanol, (S)-(−)-5,5′,6,6′,7,7′,8,8′-octahydro-1,1′-2-naphthol or Salen ligand;
the metal ligand is metal-Salen ligand, metal-BINOL ligand or (1R,2R)-(+)-N,N′-Di-p-toluenesulfonyl-1,2-cyclohexanediamine-metal ligand.

76. The method according to claim 75, wherein the amount of substance of R-1,1′-Bi-2-naphthol is 0.01 to 0.9 times that of compound (VIa); preferably, the amount of substance of R-1,1′-Bi-2-naphthol is 0.05 to 0.2 times that of compound (VIa);
the amount of substance of Salen ligand is 1.0 time or less that of compound (VIa); preferably, the amount of substance of Salen ligand is 0.2 times or less that of compound (VIa).

77. The method according to claim 73, wherein the amount of substance of isopropyl titanate is 0.5 to 8.0 times that of compound (VIa); preferably, the amount of substance of isopropyl titanate is 1.0 to 5.0 times that of compound (VIa); preferably, the amount of substance of isopropyl titanate is 1.4 to 4.0 times that of compound (VIa); preferably, the amount of substance of isopropyl titanate is 1.4 to 2.0 times that of compound (VIa); preferably, the amount of substance of isopropyl titanate is 2.0 to 4.0 times that of compound (VIa); preferably, the amount of substance of isopropyl titanate is 3.9 times that of compound (VIa); preferably, the amount of substance of isopropyl titanate is 1.4, 2.0, 3.0 or 4.0 times that of compound (VIa).

78. The method according to claim 73, wherein the amount of substance of methylating reagent 1 is 3.0 to 6.0 times that of compound (VIa); preferably, the amount of substance of methylating reagent 1 is 4.0 to 6.0 times that of compound (VIa).

79. The method according to claim 78, wherein the amount of substance of dimethyl zinc is 3.0 to 6.0 times that of compound (VIa); preferably, the amount of substance of dimethyl zinc is 4.0 to 6.0 times that of compound (VIa);
the amount of substance of methylmagnesium bromide is 3.0 to 6.0 times that of compound (VIa); preferably, the amount of substance of methylmagnesium bromide is 4.0 to 6.0 times that of compound (VIa); preferably, the amount of substance of methylmagnesium bromide is 4.0 to 5.0 times that of compound (VIa).

80. The method according to claim 73, wherein the reaction of compound (VIa) and the methylating reagent 1 is carried out in an organic solvent, and the organic solvent is dichloromethane, tetrahydrofuran, 2-methyltetrahydrofuran, methyl tert-butyl ether, toluene, o-xylene, p-xylene, meta-xylene or any combination thereof.

81. The method according to claim 73, wherein the reaction temperature of the reaction of compound (VIa) and the methylating reagent 1 is 10° C.˜ 40° C.; preferably, the reaction temperature is 20° C.˜ 35° C.; preferably, the reaction temperature is 20° C.˜32° C.;
preferably, the reaction temperature is 20° C.˜30° C.

82. The method according to claim 73, wherein, in the reaction of compound (VIa) and the methylating reagent 1, compound (VIa) is added by dropping, wherein the temperature of the reaction system during the dropping process is −20° C.˜ 25° C.; preferably, the temperature of the reaction system during the dropping process is −10° C. 0° C.; preferably, the temperature of the reaction system during the dropping process is −5° C.˜0° C.

83. The method according to claim 73, wherein the compound (VIa) can be prepared by the following method:
step a: reacting compound (VIIIa) in the presence of alkaline reagent 1 through a hydrolysis reaction to obtain compound (VIIa), 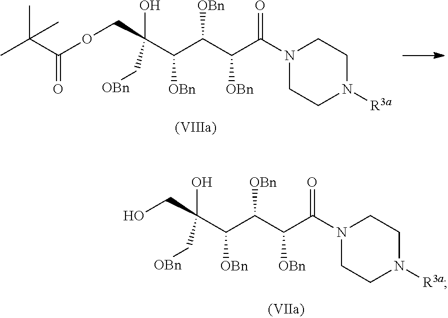
step b: reacting compound (VIIa) in the presence of oxidant 1 through an oxidation reaction to obtain compound (VIa),

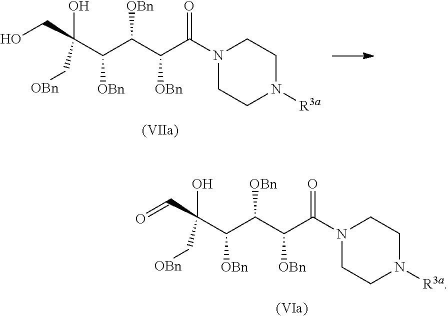

84. The method according to claim 83, wherein the alkaline reagent 1 in step a is sodium methoxide, sodium ethoxide, sodium tert-butoxide, potassium tert-butoxide, sodium carbonate, potassium carbonate, cesium carbonate, bicarbonate sodium, sodium hydroxide or potassium hydroxide;
the solvent used in step a is dichloromethane, toluene, dichloroethane, methyl tert-butyl ether, xylene, dimethyl sulfoxide, methanol, ethanol, tetrahydrofuran, 2-methyltetrahydrofuran or any combination thereof;
the reaction temperature in step a is −15° C.˜30° C.; preferably, the reaction temperature in step a is −15° C.˜5° C.; preferably, the reaction temperature in step a is −15° C.˜0° C.

85. The method according to claim 83, wherein in step a, the alkaline reagent 1 is sodium methoxide, sodium ethoxide, sodium tert-butoxide or potassium tert-butoxide; the solvent is toluene or a mixed solvent of toluene and dimethyl sulfoxide; the reaction temperature is −15° C.˜0° C.

86. The method according to claim 85, wherein the solvent used in step a is a mixed solvent of toluene and dimethyl sulfoxide, wherein the volume ratio of toluene and dimethyl sulfoxide is (20:1)˜(25:1).

87. The method according to claim 84, wherein the oxidant 1 in step b is sodium hypochlorite, 2,2,6,6-tetramethylpiperidine oxide, sulfur trioxide pyridine, oxygen, ozone, Dess-Martin oxidizer, iron nitrate, 2-iodoyl benzoic acid or iodine;
the solvent used in step b is toluene, methyl tert-butyl ether, tetrahydrofuran, methyltetrahydrofuran, dimethyl sulfoxide, dichloromethane, dichloroethane or any combination thereof;
the reaction temperature of step b is −10° C.˜30° C.; preferably, the reaction temperature of step b is −5° C.˜5° C.; preferably, the reaction temperature of step b is −5° C., −5° C.˜0° C. or 0° C.˜5° C.

88. The method according to claim 84, wherein step b is optionally carried out in the presence of an alkaline reagent b, and the alkaline reagent b is N,N-diisopropylethylamine, triethylamine, pyridine, 4-dimethylaminopyridine, N-methylmorpholine, 1,8-diazabicycloundec-7-ene or tetramethylethylenediamine.

89. The method according to claim 84 further comprises a purification method of compound (VIIIa), wherein the purification method comprises: adding the material containing compound (VIIIa) into solvent A, and then adding solvent B to precipitate a solid compound (VIIIa);
wherein, the solvent A is methanol, ethanol, isopropanol, n-butanol, tert-butanol, acetone, toluene, xylene, ethyl acetate, dichloromethane, methyl tert-butyl ether, diethyl ether, isopropyl ether, anisole or any combination thereof; and the solvent B is n-heptane, n-hexane, cyclohexane, petroleum ether, water or any combination thereof.

90. The method according to claim 89, wherein the solvent A is toluene, and the solvent B is n-heptane; or the solvent A is toluene, and the solvent B is n-hexane; or the solvent A is ethanol, and the solvent B is water; or the solvent A is isopropanol, and the solvent B is water; or the solvent A is tert-butanol, and the solvent B is water; or the solvent A is acetone, and the solvent B is water; or the solvent A is ethanol, and the solvent B is n-heptane; or the solvent A is ethanol, and the solvent B is n-hexane; or the solvent A is ethanol, and the solvent B is cyclohexane; or the solvent A is ethyl acetate, and the solvent B is n-heptane; or the solvent A is ethyl acetate, and the solvent B is n-hexane; or the solvent A is methyl tert-butyl ether, and the solvent B is n-heptane.

91. The method according to claim 90, wherein the volume ratio of toluene and n-heptane is (1:3)˜(1:8); preferably, the volume ratio of toluene and n-heptane is (1:3)˜(1:5);
the volume ratio of toluene and n-hexane is (1:3)˜(1:8); preferably, the volume ratio of toluene and n-hexane is (1:5)˜(1:8);
the volume ratio of ethyl acetate and n-heptane is (1:1)˜(1:5); the volume ratio of ethyl acetate and n-heptane is (1:2)˜(1:3);
the volume ratio of the methyl tert-butyl ether and n-heptane is (1:1)˜(1:5); the volume ratio of the methyl tert-butyl ether and n-heptane is (1:2)˜(1:3);
the volume ratio of acetone and water is (1:1)˜(10:1); preferably, the volume ratio of acetone and water is (4:1)˜(6:1);
the volume ratio of ethanol and water is (1:1)˜(3:1); the volume ratio of isopropanol and water is (1:1)˜(3:1); the volume ratio of ethanol and n-heptane is (1:1)˜(3:1);
the volume ratio of ethanol and n-hexane is (1:1)˜(3:1); the volume ratio of ethanol and cyclohexane is (1:1)˜(3:1).

92. The method according to claim 89, wherein the crystallization temperature of the solid compound (VIIIa) is 40° C.˜10° C.; preferably, the crystallization temperature of the solid compound (VIIIa) is 30° C.˜10° C.

93. A method for preparing compound (Vc) comprising: reacting compound (VIb) with methylating reagent 1 through an addition reaction in the presence of isopropyl titanate to obtain compound (Vb); and continuing to react compound (Vb) to remove the tert-butoxycarbonyl protecting group to obtain the compound (Vc); 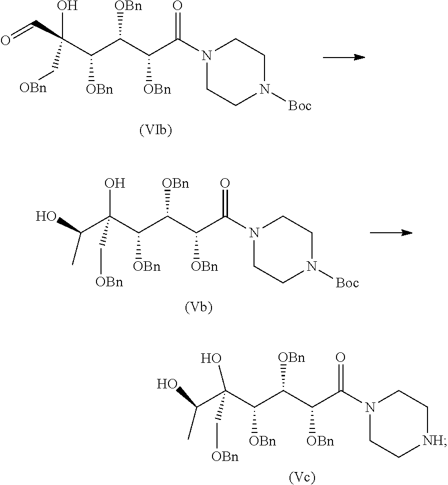
wherein the amount of substance of isopropyl titanate is 1.0 to 5.0 times that of compound (VIb); or the amount of substance of isopropyl titanate is 1.4 to 4.0 times that of compound (VIb); or the amount of substance of isopropyl titanate is 2.0 to 4.0 times that of compound (VIb);
in the addition reaction, compound (VIb) is added by dropping, and the temperature of the reaction system during the dropping process is −10° C.˜0° C.;
the method for removing the tert-butoxycarbonyl group comprises: directly increasing the temperature of the addition reaction to 25° C.˜50° C.; preferably, directly increasing the temperature of the addition reaction to 30° C.˜40° C.; preferably, directly increasing the temperature of the addition reaction to 30° C.˜35° C.

94. The method according to claim 93, wherein the methylating reagent 1 is methylmagnesium bromide or dimethyl zinc; wherein the amount of substance of methylmagnesium bromide is 4.0 to 5.0 times that of compound (VIb); and the amount of substance of dimethyl zinc is 4.0 to 6.0 times that of compound (VIb).

95. The method according to claim 93, wherein the amount of substance of isopropyl titanate is 2.0 to 4.0 times that of compound (VIb), the methylating reagent 1 is methylmagnesium bromide, and the amount of substance of methylmagnesium bromide is 4.0 to 5.0 times that of compound (VIb); or
the amount of substance of isopropyl titanate is 1.4 to 2.0 times that of compound (VIb), the methylating reagent 1 is dimethyl zinc, and the amount of substance of dimethyl zinc is 4.0 to 6.0 times that of compound (VIb).

96. The method according to claim 93, wherein the solvent of the addition reaction of compound (VIb) and methylating reagent 1 is dichloromethane, tetrahydrofuran, 2-methyltetrahydrofuran, methyl tert-butyl ether, toluene, o-xylene, p-xylene, meta-xylene or any combination thereof.

97. The method according to claim 93, wherein the addition reaction is optionally carried out in the presence of a chiral ligand 1; wherein the chiral ligand 1 is R-1,1′-Bi-2-naphthol, Salen ligand, metal-Salen ligand or metal-BINOL ligand;
the amount of substance of R-1,1′-Bi-2-naphthol is 0.05 to 0.2 times that of compound (VIb); the amount of substance of Salen ligand is 0.2 times or less that of compound (VIb).

98. A method for preparing compound (I) comprising the steps of:
step (A): reacting compound (III) or a salt thereof with compound (IV) through a coupling reaction to obtain compound (II), 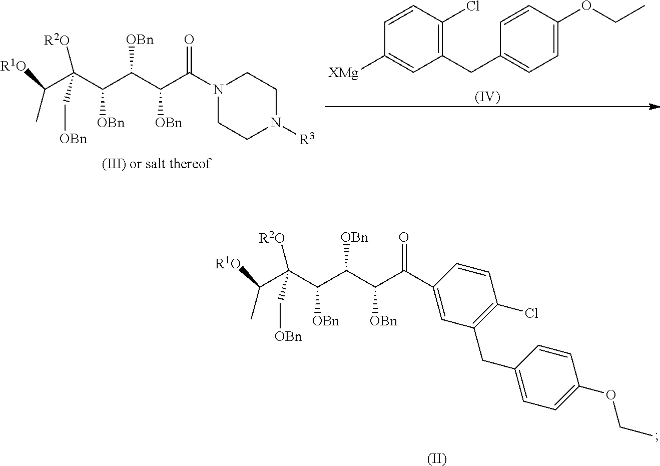
step (B): reacting compound (II) through hydrogenation reduction and ring closure under acidic condition to obtain compound (I), 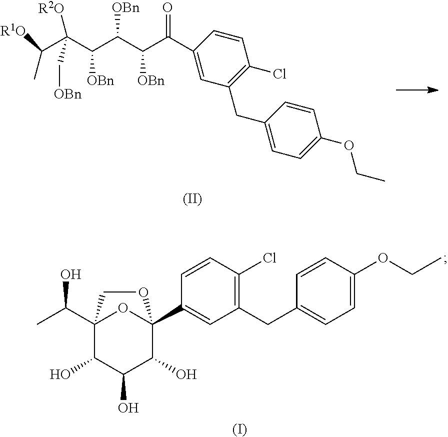
wherein,
X is Cl, Br or I;
R³ is benzyl, C₂₋₈ alkyl, C₄₋₈ alkenyl, allyl, phenyl, p-toluenesulfonyl, benzenesulfonyl, 4-bromobenzenesulfonyl, 4-nitrophenyl, 1,3-dichlorophenyl, triphenylmethyl, bis(4-methoxyphenyl) (phenyl) methyl, diphenylmethyl, N,N-diphenylaminoacyl, pyridyl, benzylsulfonyl, imidazolyl, N,N-dimethylaminosulfonyl, N,N-dimethylaminoacyl or 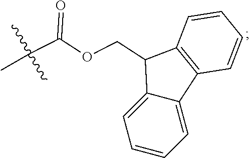 [SMILES: *C(=O)OCC1c2ccccc2-c2ccccc21]
and
each R¹ and R² is independently benzyl, triphenylmethyl, p-methoxybenzyl, tert-butyldimethylsilyl, trimethylsilyl, tert-butyldiphenylsilyl, triethyl silyl, triisopropylsilyl, benzyloxycarbonyl, 2-(trimethylsilyl)ethoxymethyl, dihydropyranyl, bromopropenyl, ethylformyl, acetyl or benzoyl;
or, R¹ and R² together with —OCHCHO— to which they are attached form

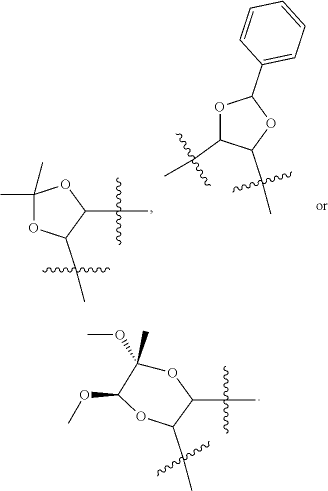

99. The method according to claim 98, wherein the acidic condition in step (B) refers to the reaction reacts in the presence of an acid, and the acid is hydrochloric acid, perchloric acid, sulfuric acid, nitric acid, formic acid or acetic acid;
the hydrogenation reduction in step (B) is carried out in the presence of a catalyst, and the catalyst is palladium/carbon, palladium hydroxide/carbon, platinum/carbon, Raney nickel or palladium chloride; wherein, the mass ratio of the catalyst and compound (II) is (0.02:1)˜(0.8:1); preferably, the mass ratio of the catalyst and compound (II) is (0.1:1)˜(0.6:1); preferably, the mass ratio of the catalyst and compound (II) is (0.05:1) to (0.2:1).

100. The method according to claim 98, wherein, in step (A), the amount of substance of compound (IV) is 1.0 to 1.5 times that of compound (III) or a salt thereof;
preferably, in step (A), the amount of substance of compound (IV) is 1.1 to 1.3 times that of compound (III) or a salt thereof;
the reaction solvent in step (A) is tetrahydrofuran, 2-methyltetrahydrofuran, methyl tert-butyl ether, toluene, dichloromethane or any combination thereof;
the reaction temperature in step (A) is 10° C.˜ 40° C.; preferably, the reaction temperature in step (A) is 20° C.˜ 30° C.

101. The method according to claim 98, wherein compound (III) is prepared by the following method A or method B,
method A, 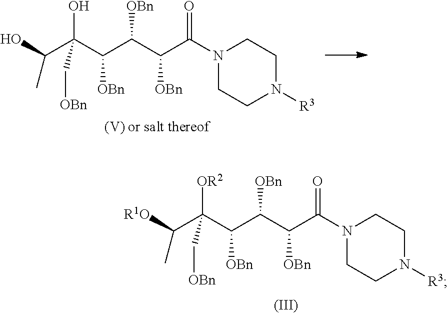
wherein, the method A includes the step of: reacting compound (V) or a salt thereof with hydroxyl protecting reagent 1 to obtain compound (III);
method B,
step 1: reacting compound (Vc) with acid 1 to obtain compound (Vd), 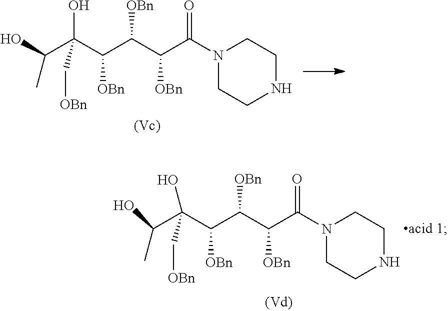
step 2: reacting compound (Vd) with hydroxyl protecting reagent 2 to obtain compound (Ve), 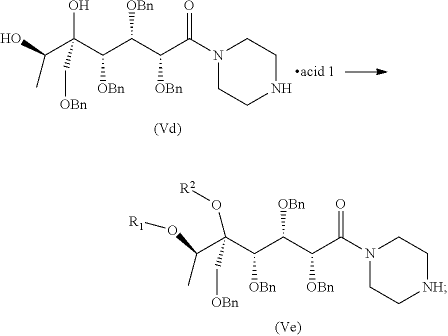
step 3: reacting compound (Ve) in the presence of acid 2 to obtain compound (Vf), 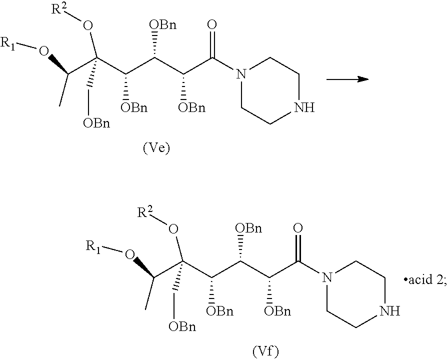
step 4: reacting compound (Vf) with compound R³X¹ to obtain compound 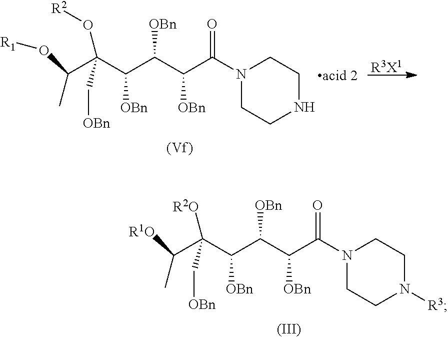
wherein, X¹ is Cl, Br or I; and
each acid 1 and acid 2 is independently sulfuric acid, hydrochloric acid, nitric acid, phosphoric acid, oxalic acid, pivalic acid, methanesulfonic acid, acetic acid, formic acid, benzoic acid, p-toluenesulfonic acid, citric acid, cinnamic acid, tartaric acid, malic acid, salicylic acid, succinic acid or caffeic acid.

102. The method according to claim 101, wherein the acid 1 is used to adjust the pH of the solution; wherein after acid 1 is added, the pH of the solution is 1.0˜7.0; preferably, after acid 1 is added, the pH of the solution is 3.0˜ 7.0; preferably, after acid 1 is added, the pH of the solution is 5.5˜ 7.0;
the hydroxyl protecting reagent 1 and the hydroxyl protecting reagent 2 are each independently 2,2-dimethoxypropane, benzaldehyde dimethyl acetal, trimethylchlorosilane or tert-butyldimethylchlorosilane;
the reaction solvent of the method A and the reaction solvent in step 2 of method B are each independently toluene, 1,2-dichloroethane, dichloroethane, methyl tert-butyl ether, isopropyl ether, tetrahydrofuran, 2-methyltetrahydrofuran or any combination thereof; and
the reaction temperature of the method A and the reaction temperature of step 2 of the method B are each independently 0° C.˜ 40° C.; preferably, the reaction temperature of the method A and the reaction temperature of step 2 of the method B are each independently 10° C.˜ 40° C.; preferably, the reaction temperature of the method A and the reaction temperature of step 2 of the method B are each independently 8° C.˜ 30° C.;
the amount of substance of acid 2 is 0.5 to 2.0 times that of compound (Ve); preferably, the amount of substance of acid 2 is 0.5 to 1.2 times that of compound (Ve).

103. The method according to claim 101, wherein the reactions of the method A and the step 2 of the method B are each independently and optionally carried out in the presence of methanesulfonic acid.

104. A compound having one of the following structures or a salt thereof: 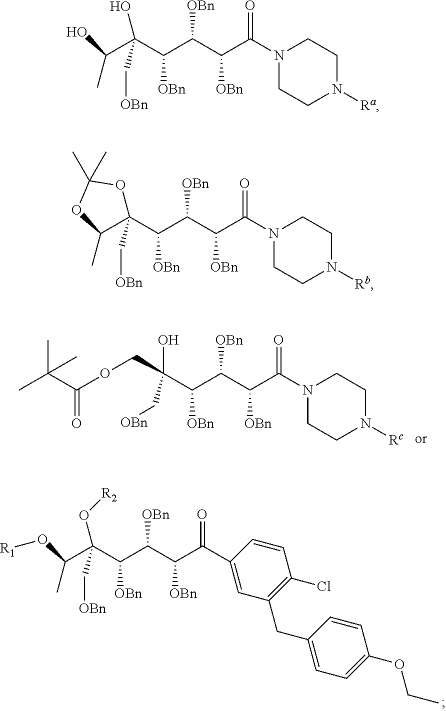
wherein, each R^{a}, R^{b} and R^{c} is independently H, C₂₋₈ alkyl, C₄₋₈ alkenyl, allyl, phenyl, benzyl, p-toluenesulfonyl, benzenesulfonyl, 4-bromobenzenesulfonyl, 4-nitrophenyl, 1,3-dichlorophenyl, tert-butoxycarbonyl, triphenylmethyl, bis(4-methoxyphenyl)(phenyl)methyl, diphenylmethyl, N,N-diphenylaminoacyl, pyridyl, benzylsulfonyl, imidazolyl, N,N-dimethylaminosulfonyl, N,N-dimethylaminoacyl or 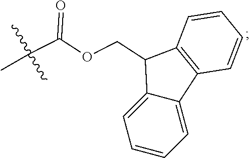 [SMILES: *C(=O)OCC1c2ccccc2-c2ccccc21]
each R¹ and R² is independently benzyl, triphenylmethyl, p-methoxybenzyl, tert-butyldimethylsilyl, trimethylsilyl, tert-butyldiphenylsilyl, triethyl silyl, triisopropylsilyl, benzyloxycarbonyl, 2-(trimethylsilyl)ethoxymethyl, dihydropyranyl, bromopropenyl, ethylformyl, acetyl or benzoyl,
or, R¹ and R² together with —OCHCHO— to which they are attached form

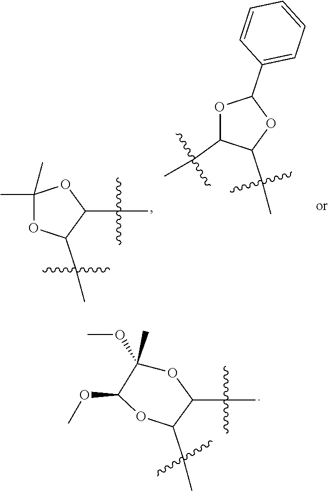

105. A method for preparing compound (VIII) comprising the step of: reacting compound (IX) and the Grignard reagent obtained by the Grignard exchange of iodomethyl pivalate and isopropyl magnesium chloride lithium chloride through an addition reaction to obtain compound (VIII), 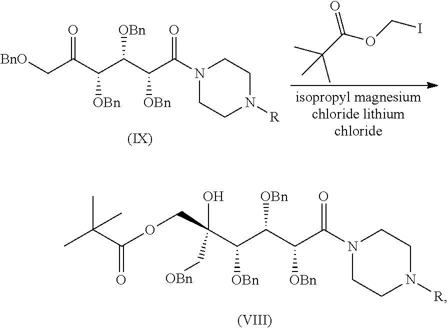
wherein, R is H, ethyl, n-propyl, n-butyl, tert-butyl, 2,2-dimethylpropyl, allyl, phenyl, benzyl, p-toluenesulfonyl, benzenesulfonyl, 4-bromobenzenesulfonyl, 4-nitrophenyl, 1,3-dichlorophenyl, tert-butoxycarbonyl, triphenylmethyl, bis(4-methoxyphenyl)benzyl, diphenylmethyl, N,N-diphenylaminoacyl, pyridyl, benzylsulfonyl, imidazolyl, N,N-dimethylaminosulfonyl, N,N-dimethylaminoacyl or 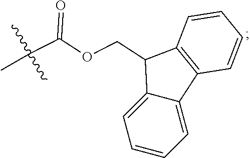
wherein, the method comprises a purification method of compound (VIII), and the purification method comprises: adding the material containing compound (VIII) into solvent A, and then adding solvent B to precipitate a solid compound (VIII); wherein the solvent A is methanol, ethanol, isopropanol, n-butanol, tert-butanol, acetone, toluene, xylene, ethyl acetate, dichloromethane, methyl tert-butyl ether, diethyl ether, isopropyl ether, anisole or any combination thereof; and the solvent B is n-heptane, n-hexane, cyclohexane, petroleum ether, water or any combination thereof.

106. The method according to claim 105, wherein the solvent A is toluene, the solvent B is n-heptane, and the volume ratio of toluene and n-heptane is (1:3)˜(1:10); preferably, the volume ratio of toluene and n-heptane is (1:3)˜(1:8); preferably, the volume ratio of toluene and n-heptane is (1:3)˜(1:5); preferably, the volume ratio of toluene and n-heptane is (1:5)˜(1:8); or
the solvent A is acetone, and the solvent B is water, wherein the volume ratio of acetone and water is (1:1)˜(10:1); preferably, the volume ratio of acetone and water is (2:1)˜(8:1); preferably, the volume ratio of acetone and water is (4:1)˜(6:1); or
the solvent A is ethyl acetate, and the solvent B is n-heptane, wherein the volume ratio of ethyl acetate and n-heptane is (1:1)˜(1:10); preferably, the volume ratio of ethyl acetate and n-heptane is (1:1)˜ (1:5); preferably, the volume ratio of ethyl acetate and n-heptane is (1:2)˜ (1:3); or
the solvent A is methyl tert-butyl ether, and the solvent B is n-heptane, wherein the volume ratio of the methyl tert-butyl ether and n-heptane is (1:3)˜ (1:10).

107. Crystal form A of compound (VIIIb), 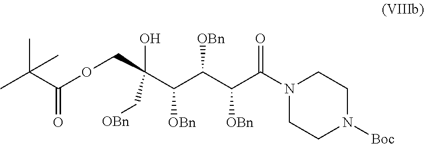 [SMILES: CC(C)(C)OC(=O)N1CCN(C(=O)[C@H](OCc2ccccc2)[C@@H](OCc2ccccc2)[C@H](OCc2ccccc2)[C@@](O)(COCc2ccccc2)COC(=O)C(C)(C)C)CC1]
wherein the X-ray powder diffraction pattern of the crystal form A has diffraction peaks at the following 2θ angles: 6.52°+0.2°, 6.89°+0.2°, 9.25°+0.2°, 9.66°±0.2°, 16.18°±0.2°, 18.44°±0.2°, 18.56°±0.2°, 21.87°±0.2°, 27.58°±0.2°; or
the X-ray powder diffraction pattern of the crystal form A has diffraction peaks at the following 2θ angles: 6.52°+0.2°, 6.89°+0.2°, 9.25°±0.2°, 9.66°±0.2°, 13.72°±0.2°, 16.18°±0.2°, 18.44°±0.2°, 18.56°±0.2°, 18.86°±0.2°, 19.33°±0.2°, 20.53°±0.2°, 21.39°±0.2°, 21.87°±0.2°, 23.52°±0.2, 23.95°±0.2°, 24.38°±0.2°, 26.07°±0.2°, 27.58°±0.2°; or
the X-ray powder diffraction pattern of the crystal form A has diffraction peaks at the following 2θ angles: 6.52°+0.2°, 6.89°+0.2°, 9.25°+0.2°, 9.66°+0.2°, 13.72°±0.2°, 14.44°±0.2°, 14.97°±0.2°, 15.30°±0.2°, 16.18°±0.2°, 16.95°±0.2°, 17.19°±0.2°, 18.21°±0.2°, 18.44°±0.2°, 18.56°±0.2°, 18.86°±0.2°, 19.33°±0.2°, 19.56°±0.2°, 20.13°±0.2°, 20.53°±0.2°, 20.98°±0.2°, 21.39°±0.2°, 21.87°±0.2°, 22.38°±0.2°, 22.67°±0.2°, 23.12°±0.2°, 23.52°±0.2, 23.95°±0.2°, 24.38°±0.2°, 24.88°±0.2°, 26.07°±0.2°, 27.58°±0.2°, 29.11°±0.2°, 30.54°±0.2°, 31.07°±0.2°, 31.74°±0.2°, 32.76°±0.2°, 34.69°±0.2°, 35.06°±0.2°, 39.58°±0.2°, 39.89°±0.2°, 40.51°±0.2°; or
the crystal form A has an X-ray powder diffraction pattern substantially as shown in FIG. 1.
